(19) United States
(12) Patent Application Publication
(10) Pub. No.: US 2023/0257715 A1
(43) Pub. Date: Aug. 17, 2023

(54) COMPOSITIONS AND METHODS FOR REGULATING ENTEROENDOCRINE CELL DIFFERENTIATION AND USES THEREOF

(71) Applicant: The Children's Medical Center Corporation, Boston, MA (US)

(72) Inventors: David T. BREAULT, Boston, MA (US); Daniel R. ZEVE, Boston, MA (US)

(73) Assignee: The Children's Medical Center Corporation, Boston, MA (US)

(21) Appl. No.: 18/011,089

(22) PCT Filed: Jun. 16, 2021
(86) PCT No.: PCT/US21/37592

§ 371 Date: Dec. 16, 2022

Related U.S. Application Data
(60) Provisional application No. 63/040,304, filed on Jun. 17, 2020.

(51) Int. Cl. C12N 5/071; A61K 35/38

(52) U.S. Cl. CPC C12N 5/0679; A61K 35/38; C12N 2501/60; C12N 2501/727; C12N 2501/415; C12N 2506/23; C12N 2513/00

(57) ABSTRACT

The present invention features, in some embodiments, compositions comprising ex vivo or in vitro generated functional enteroendocrine (EE) cells, or enteroids, rectoids, or organoids comprising functional enteroendocrine cells, and methods of obtaining and using such cells for treating metabolic and gastrointestinal diseases, disorders, or conditions.

### CROSS-REFERENCE TO RELATED APPLICATIONS

This application claims the benefit of the following U.S. Provisional Application No. 63/040,304, filed on Jun. 17, 2020, the entire contents of which are incorporated herein by reference.

### STATEMENT OF RIGHTS TO INVENTIONS MADE UNDER FEDERALLY SPONSORED RESEARCH

This invention was made with government support under Grant Nos. T32DK769937, K12DK09472109, HL095722, UC4DK104165, and DK119488 awarded by the National Institutes of Health. The government has certain rights in the invention.

### BACKGROUND OF THE INVENTION

The gastrointestinal tissues of the stomach and intestines, for example, contain large numbers of adult stem cells or progenitor cells that continuously produce epithelial cells, including enteroendocrine cells. These enteroendocrine (EE) cells are found throughout the gastrointestinal (GI) tract and represent the most abundant hormone-producing cell type in mammals. EE cells, as a whole, secrete a large variety of hormones, including glucose-dependent insulinotropic polypeptide (GIP), cholecystokinin (CCK), serotonin (5HT), somatostatin (SST), peptide YY (PYY), and glucagon-like peptide-1 (GLP-1), among others. In response to physiological and nutritional cues, EE cells, through the production of these various hormones, are responsible for regulating multiple aspects of GI activity and nutritional homeostasis. Because of this, EE cells have been implicated in the pathogenesis of GI diseases such as irritable bowel syndrome and inflammatory bowel disease, as well as metabolic diseases such as obesity or type 2 diabetes.

EE cells are plurihormonal, i.e., they synthesize and secrete multiple peptide hormones, and are also important regulators of energy homeostasis and gastrointestinal function. EE cells are also defined by expression of the specific hormone each produces and their location along the GI tract. These cells also express multiple neuroendocrine secretory proteins, including Chromogranin A (CHGA), some of which are more specific to certain EE cell types than others. Further, multiple transcription factors are critical for EE cell differentiation and function, including neurogenin 3 (NEUROG3) and neuronal differentiation 1 (NEUROD1). Pancreatic and duodenal homeobox 1 (PDX1) has also been shown to regulate gene expression in a subpopulation of EE cells located within the duodenum. In patients with metabolic and gastrointestinal diseases, EE cell dysfunction has been observed. However, existing protocols are extremely limited in their ability to induce protein expression for endocrine cell markers and pose challenges in converting human intestinal stem cells (ISCs) into functional EE cells ex vivo. Similar to other mature intestinal epithelial cells, EE cells are derived from ISCs, which reside within the crypts of Lieberkuhn. In recent years, much progress has been made to understand the mechanisms underlying ISC self-renewal and differentiation using 3D-organoid culture. Due to the limitations imposed by present protocols, progress in elucidating the role of EE cells in disease pathogenesis and in harnessing their therapeutic potential has been restricted. Using combinations of growth factors and small molecules targeting specific transcriptional regulators and signaling pathways, intestinal organoids can either be maintained predominantly as ISCs or differentiated into mature intestinal cells of either the absorptive or secretory lineages. For example, maintenance of ISC self-renewal requires activation of canonical Wnt signaling using WNT3a and R-spondin, suppression of bone morphogenic protein (BMP) signaling using Noggin, and inhibition of p38 MAPK signaling using the small molecule SB202190. By altering these pathways along with transcriptional regulators, strategies have begun to emerge to direct ISC differentiation into mature intestinal epithelial cell types.

The available human EE cell directed differentiation protocols, which involve removing Wnt ligands and inhibition of Notch signaling. For example, removal of Wnt3a and the p38 mitogen-activated protein kinase (MAPK) inhibitor, SB202190, from growth media, and addition of the Notch inhibitor, DAPT, are not sufficient to produce desired levels of enteroendocrin cells or express their hormones. Yet, in contrast to mice, human EE cells do not differentiate in the presence of p38 MAPK inhibitors. While strong mRNA expression of multiple mature EE markers has been observed, limited data exist showing robust protein expression of EE cell markers using other differentiation protocol. Presently available methods also typically involve direct genetic modifications, but oftentimes this results in mutagenesis.

Therefore, there is a need in the art to design robust and improved methods of human EE cell differentiation, as well as therapeutic methods utilizing these cells, particularly in those suffering from conditions and diseases having EE cell dysfunction. Unfortunately, there are few existing therapeutic options to target the processes evoked by enterodendocrine cell dysfunction.

### SUMMARY OF THE INVENTION

As described below, the present invention features in some embodiments, methods of obtaining and compositions comprising ex vivo or in vitro generated functional enteroendocrine (EE) cells, or organoids (e.g., enteroids or rectoids) comprising functional enteroendocrine cells, and methods of using such cells for treating metabolic and gastrointestinal diseases, disorders, or conditions (e.g., obesity, type II diabetes mellitus, irritable bowel syndrome (IBS), inflammatory bowel disease (IBD), Crohn's disease, and ulcerative colitis).

One aspect of the disclosure provides for an in vitro generated three-dimensional gastrointestinal organoid, including but not limited to, enteroids or rectoids, derived from an induced human gastrointestinal stem cell (GISC), the organoid comprising functional human enteroendocrine cells. One aspect is directed to a gastrointestinal organoid, where the organoid may include but is not limited to an enteroid or a rectoid, and the organoid comprises functional human enteroendocrine cells. A further aspect provides such gastrointestinal organoids that are three-dimensional. Another aspect relates to such gastrointestinal organoids derived from an induced human gastrointestinal stem cell (GISC), where the gastrointestinal organoid is three-dimensional and generated in vitro. In some aspects, the organoid comprises enteroendocrine cells that secrete hormones, such as but not limited to cholecystokinin (CCK), gastrin, ghrelin, glucagon-like peptide-1 (GLP-1), GLP-2, glucose-dependent insulinotropic polypeptide (GIP), histamine, leptin, motilin, neurotensin, oxyntomodulin, peptide YY (PYY), secretin, serotonin (5HT), and somatostatin (SST). Other aspects are directed to such organoids of the disclosure comprising enteroendocrine cells that secrete hormones including but not limited to: CCK, GLP-1, GIP, 5HT, SST, andPYY. These enteroendocrine cells are functional and behave as those found in subjects not suffering from any enteroendocrine cell dysfunction disease. The organoid may, in another aspect, comprise an enteroendocrine (EE) marker or lineage marker selected from at least one of: ALP1, ATOH1, CCK, CHGA, GCG, GIP, LGR5, MUC2, NEUROD1, NEUROG3, PAX4, PYY, and SST, or combinations thereof. In other embodiments, the organoid may comprise an EE marker (including but not limited to, lineage markers) selected from, but not limited to, enteroendocrine lineage markers (e.g., chromogranin A (CHGA), mucin 2 (MUC2), lysozyme (LYZ), ALP1, LGR5), enteroendocrine cell differentiation and function transcription factors (e.g., PDX1, neurogenin 3 (NEUROG3), neuronal differentiation 1 (NEUROD1), atonal BHLH transcription factor 1 (ATOH1)), hormones (e.g., v, glucose-dependent insulinotropic polypeptide (GIP)), or any combinations thereof.

Another aspect provides a method of enteroendocrine cell differentiation, comprising culturing gastrointestinal stem cells in a media and adding at least one modulating agent, wherein said modulating agent may be selected from a GATA4 activator (e.g., Rim), putative GATA4 activator (e.g., Rim), or cannabinoid type 1 receptor (CB1) inverse agonist (e.g., Rim), a direct or indirect PDX1 activator (e.g., SP600125), a JNK inhibitor (e.g., SP600125), a FOXO1 inhibitor (e.g., AS1842856), or combinations thereof, said modulating agent is in an amount effective to induce differentiation of the gastrointestinal stem cells, thereby resulting in enteroendocrine cell differentiation. The modulating agent may comprise, in one aspect, a GATA4 activator and either a PDX1 activator or a JNK inhibitor; and/or in a further aspect a FOXO1 inhibitor. Certain aspects of the disclosure provide a method where the modulating agent that is a FOXO1 inhibitor may be administered alone or in combination with another modulating agent for a predetermined number of days, such as 1-21 days (e.g., 1-20, 1-14, 1-12, 1-7, 1-5), where in some aspects, the FOXO1 inhibitor is administered without any other modulating agent, i.e., alone. Another aspect provides for administration of additional modulating agents following the administration of FOXO1 inhibitor for a predetermined number of days. For example, the method further comprises administering for any number of days ranging 1-21 days (e.g., 1-20, 1-14, 1-12, 1-7, 1-5) a GATA4 activator and either a PDX1 activator or a JNK inhibitor, after FOXO1 is administered for the predetermined number of days, where the second administrating step does not contain the FOXO1 inhibitor modulating agent. An alternative aspect comprises the second administrating step where the at least one modulating agent comprises a combination of a FOXO1 inhibitor, a GATA4 activator, and either a PDX1 activator or a JNK inhibitor, after FOXO1 is administered alone for the predetermined number of days ranging 1-21 days (e.g., 1-20, 1-14, 1-12, 1-7, 1-5).

A further aspect of the disclosure provides methods utilizing a media, such as a growth media and/or a differentiation media. The growth media (GM) may contain, for example, conditioned media containing Wnt3a, noggin, and R-spondin 3 (e.g., L-WRN conditioned media (50% v/v)); mammalian cell culture media with high concentrations of glucose, amino acids, and vitamins with additional nutrients (e.g., DMEM/F12 (45% v/v)); supplements (e.g., GlutaMax™ (1% v/v); N-2 Supplement (1% v/v); B-27 Supplement (1% v/v)); buffering agent (e.g., HEPES (10 mM)); antibiotic/antimicrobial (e.g., Primocin (100 μg/mL); Normocin (100 μg/mL)); small molecule modulating agent (e.g., TGF-βR inhibitor, A83-01 (500 nM); p38 MAPK inhibitor, SB202190 (10 μM)); antioxidant (e.g., N-Acetyl-cysteine (500 μM)); proliferating and/or differentiating agent (e.g., recombinant EGF (50 ng/mL); Nicotinamide (10 mM)); hormone (e.g., Human [Leu15] Gastrin I (10 nM). The culturing in growth media may occur for a period of time greater than or equal to one day (e.g., 2 days, 3 days, 4 days, 5 days, 6 days, 7 days).

In other aspects, the media may be a differentiation media containing, for example, conditioned media containing Wnt3a, noggin, and R-spondin 3 (e.g., L-WRN conditioned media (50% v/v)); mammalian cell culture media with high concentrations of glucose, amino acids, and vitamins with additional nutrients (e.g., DMEM/F12 (45% v/v)); supplements (e.g., GlutaMax™ (1% v/v); N-2 Supplement (1% v/v); B-27 Supplement (1% v/v)); buffering agent (e.g., HEPES (10 mM)); antibiotic/antimicrobial (e.g., Primocin (100 μg/mL); Normocin (100 μg/mL)); small molecule modulating agent (e.g., TGF-βR inhibitor, A83-01 (500 nM); γ-secretase or Notch signaling inhibitor, DAPT (20 μM); HDAC6 inhibitor, Tubastatin-A (10 μM); MAP4K4 inhibitor, PF06260933 (6 μM); lysine-specific demethylase 1 inhibitor, Tranylcypromine (1.5 μM); a putative GATA4 activator or CB1 inverse agonist, Rimonabant (10 μM); JNK inhibitor or indirect PDX1 activator, SP600125 (10 μM); FOXO1 inhibitor, AS1842856 (100 nM)); antioxidant (e.g., N-Acetyl-cysteine (500 μM)); proliferating and/or differentiating agent (e.g., recombinant EGF (50 ng/mL); hormone (e.g., Human [Leu15] Gastrin I (10 nM); growth factor (e.g., Betacellulin (20 ng/mL). The culturing step in differentiation media may occur over a period of time greater than or equal to one day (e.g., 2 days, 3 days, 4 days, 5 days, 6 days, 7 days).

Another aspect provides for methods, where the culturing step comprises a first culturing step in a growth media and a second culturing step in a differentiation media, where the differentiation media replaces the growth media. In certain aspects, the first culturing step in a growth media may occur for a period of time greater than or equal to 1 day (e.g., 2 days, 3 days, 4 days, 5 days, 6 days, 7 days), replacing the growth media with a differentiation media, and a second culturing step (i.e., after the completion of the first culturing step) in a differentiation media may occur for a period of time greater than or equal to 1 day (e.g., 2 days, 3 days, 4 days, 5 days, 6 days, 7 days). One aspect of the methods described here, provides for at least one modulating agent in a pharmaceutical composition and at least one pharmaceutically acceptable carrier. A further aspect provides for a pharmaceutical composition comprising the at least one modulating agent and at least one pharmaceutically acceptable carrier.

In some aspects, a method of treating a subject suffering from a disease having enteroendocrine cell dysfunction may be provided and comprises: administering to the subject in need thereof, a pharmaceutical composition comprising functional enteroendocrine cells in an effective or therapeutic amount to treat the subject and at least one pharmaceutically acceptable carrier. The functional enteroendocrine cells useful in the method of treating may be obtained by any of the methods disclosed here. Non-limiting examples of diseases useful in the methods of treating disclosed here may include a metabolic and/or gastrointestinal disease (e.g., irritable bowel syndrome, inflammatory bowel disease, Crohn's disease, ulcerative colitis, obesity, diabetes, including for example, Type II diabetes mellitus).

The invention provides compositions and methods that preserve for treating metabolic and gastrointestinal diseases, disorders, or conditions by a replacement of or an increase in functional enteroendocrine cells, or enteroids or organoids comprising such cells. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, small molecule or chemical compound, or any component. In some instances, the “agent” is a “modulating agent” by which is meant an agent that affects another action. For example, a modulating agent may activate or inhibit a transcription factor as relative to a condition that does not have a modulating agent. Other instances provide a “therapeutic agent” by which is meant a small molecule or chemical compound, or functional enteroendocrine cells, or organoids (e.g., enteroids, rectoids) comprising such functional enteroendocrine cells, that may be useful in treating a subject, such as a human mammal, suffering from a metabolic and/or gastrointestinal disease comprising enteroendocrine cell dysfunction.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. “

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “carrier” is meant any substrate used for delivering an agent (e.g., peptide, nucleic acid molecule, small molecule or chemical compound, or any component, e.g., cells, modulating agents), where the substrate may improve the selectivity, effectiveness, and/or safety of the administration of the agent. The term “carrier” may also include diluents and excipients.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell (e.g., enteroendocrine cell), tissue, or organ. Non-limiting examples of diseases include metabolic and gastrointestinal diseases, such as but not limited to, obesity, type II diabetes mellitus, irritable bowel syndrome (IBS), inflammatory bowel disease (IBD), Crohn's disease, and ulcerative colitis.

By “effective amount” or “therapeutically effective amount” is meant the amount of a required agent or element used sufficient to effect beneficial or desired results, such as clinical results, and, as such, an “effective amount” depends upon the context in which it is being applied. In some embodiments, an effective amount for the treatment or prophylaxis of a disease may ameliorate the symptoms of the disease relative to an untreated subject or patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets (e.g., GATA4, JNK, PDX1, FOXO1) that are useful for the development of highly specific drugs to treat or a disorder characterized by the methods delineated herein. In addition, the methods of the invention provide a facile means to identify therapies that are safe for use in subjects, such as those suffering from an enteroendocrine cell dysfunction. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

The term “pharmaceutical composition,” as used herein, represents a composition containing an agent (e.g., a peptide, protein, nucleic acid, small molecule or chemical compound, cells) described herein formulated with a pharmaceutically acceptable carrier. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, etc.); for topical administration (e.g., as a cream, gel, lotion, or ointment); for intravenous administration (e.g., as a sterile solution free of particulate emboli and in a solvent system suitable for intravenous use); or in any other formulation described herein (see below).

As used herein, the phrase “pharmaceutically acceptable” generally safe for ingestion or contact with biologic tissues at the levels employed. Pharmaceutically acceptable is used interchangeably with physiologically compatible. It will be understood that the pharmaceutical compositions of the disclosure include nutraceutical compositions (e.g., dietary supplements) unless otherwise specified.

Unit dosage forms, also referred to as unitary dosage forms, often denote those forms of medication supplied in a manner that does not require further weighing or measuring to provide the dosage (e.g., tablet, capsule, caplet, etc.). For example, a unit dosage form may refer to a physically discrete unit suitable as a unitary dosage for human subjects and other mammals, each unit containing a predetermined quantity of active material calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, and gel cap. In certain embodiments, the compounds described herein, including crystallized forms, polymorphs, and solvates thereof, may be present in a unit dosage form.

Useful pharmaceutical carriers, excipients, and diluents for the preparation of the compositions hereof, can be solids, liquids, or gases. These include any and all solvents, dispersion media, coatings, antibacterial and antifungal agents, isotonic and absorption delaying agents and the like. The pharmaceutically acceptable carrier (e.g., diluent or excipient) does not destroy the pharmacological activity of the disclosed compound and is nontoxic when administered in doses sufficient to deliver a therapeutic amount of the agent. Thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, and aerosols. The carrier can be selected from the various oils including those of petroleum, animal, vegetable or synthetic origin, e.g., peanut oil, soybean oil, mineral oil, and sesame oil. Water, saline, aqueous dextrose, and glycols are examples of liquid carriers, particularly (when isotonic with the blood) for injectable solutions. For example, formulations for intravenous administration comprise sterile aqueous solutions of the agent, e.g., active ingredient(s), functional cells, which may be prepared by dissolving, for example, a solid active ingredient(s) in water to produce an aqueous solution, and rendering the solution sterile. Suitable pharmaceutical excipients include starch, cellulose, chitosan, talc, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions may be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for administration to the recipient.

Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of the compounds described herein.

As used herein, the terms “prevent,” “preventing,” “prevention,” “prophylactic treatment” and the like refer to reducing the probability of developing a disease in a subject, who does not have, but is at risk of or susceptible to developing a disease, such as but not limited to metabolic and/or gastrointestinal diseases (e.g., obesity, diabetes (e.g., type II diabetes mellitus), irritable bowel syndrome (IBS), inflammatory bowel disease (IBD), Crohn's disease, and ulcerative colitis). The metabolic diseases of the disclosure include, but are not limited to obesity and type 2 diabetes. Non-limiting examples of gastrointestinal diseases include: irritable bowel syndrome (IBS), inflammatory bowel disease (IBD), Crohn's disease, and ulcerative colitis.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers. For example, Tagman qPCR Primers from Life Technologies (Cat. #4331182) for identifying gene expression of various genes, including but not limited to, 18S (185), Intestinal alkaline phosphatase (ALPI), cholecystokinin (CCK), Chromogranin A (CHGA), GATA binding protein 4 (GATA4), Glucose-dependent insulinotropic polypeptide (GIP), Leucine-rich repeat-containing G-protein coupled receptor 5 (LGR5), Lysozyme (LYZ), Mucin 2 (MUC2), Neuronal differentiation 1 (NEUROD1), Neurogenin 3 (NEUROG3), paired box 4 (PAX4), and Somatostatin (SST).

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “subject” is meant any organism to which a composition and/or compound in accordance with the disclosure may be administered, e.g., for experimental, diagnostic, prophylactic, and/or therapeutic purposes. Generally, subjects may include any animal (e.g., a mammal, including, but not limited to, a human or non-human mammal, such as a rodent, primate, bovine, equine, canine, ovine, or feline). A “subject in need thereof” is typically a subject for whom it is desirable to treat a disease as described herein. For example, a subject in need thereof may seek or be in need of treatment, require treatment, be receiving treatment, may be receiving treatment in the future, or a human or non-human animal that is under care by a trained professional for a particular disease.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disease (including a disorder or condition) and/or symptoms associated therewith. It will be appreciated that, although not precluded, treating a disease, disorder, or condition does not require that the disorder, condition, or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.010% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 illustrates that base differentiation media induces CHGA expression in a Wnt-dependent manner.

FIG. 1A shows representative light microscopy of enteroids (whole well) grown in growth media (GM) for 14 days (G14) or two days in growth media (GM) followed by 12 days in differentiation media (DM; G2D12). A specific culture schematic is located above each panel, respectively. Scale bar=1 mm.

FIG. 1B provides qPCR analysis of enteroendocrine (EE) markers of enteroids grown in either G14 or G2D12 compared to whole mucosa and normalized to 18S. The dotted line denotes the expression level in whole mucosa. A representative experiment shows n=3 wells for each condition from a single enteroid line. CHGA=chromogranin A, PAX4=paired box 4, PDX1=pancreatic and duodenal homeobox 1, NEUROD1=neuronal differentiation 1, NEUROG3=neurogenin 3, CCK=cholecystokinin, SST=somatostatin, GIP=glucose-dependent insulinotropic peptide, ND=not detectable in one or more samples.

FIG. 1C and FIG. 1D illustrate representative immunofluorescence staining of CHGA (magenta) in enteroids (whole well) treated with G2D12. In FIG. 1C, DAPI stained alone (Left panel); CHGA stained alone (Center panel); and merged staining (MERGE; Right panel) are presented. The boxed portion in FIG. 1C (CHGA and Merge panels) is shown magnified in FIG. 1D that has been stained with both CHGA and DAPI (CHGA/DAPI). DNA (4′,6-diamidino-2-phenylindole (DAPI) blue). Scale bars=1 mm (FIG. 1C) and 50 μm (FIG. 1D).

FIG. 1E shows representative flow cytometry plots of CHGA-positive (CHGA+) cells from enteroids grown in either G14 (Left panel) or G2D12 (Center panel) and the percentage of CHGA+ cells per well per culture condition (i.e., G14; G2D12) (Right panel). A representative experiment shows n=3 wells from each condition from a single enteroid line.

FIG. 1F provides qPCR analysis of EE gene markers of enteroids grown in either G2D12 without Wnt (G2D12-Wnt; Left column) or with Wnt (G2D12+Wnt; Right column) compared to whole mucosa and normalized to 18S. The dotted line denotes the expression level in whole mucosa. MUC2=Mucin 2, ALPI=Intestinal alkaline phosphatase. A representative experiment shows n=3 wells from each condition from a single enteroid line. Bars show mean±SEM, **p<0.01, ***p<0.001. Each experiment was repeated with at least three different enteroid lines. Specific conditions were excluded from statistical analysis if the data from one or more samples were labeled as not detectable.

FIG. 1.1 illustrates that the base differentiation media components are important for enteroendocrine marker expression.

FIG. 1.1A shows a qPCR analysis of intestinal lineage markers of enteroids grown in either G14 (Left column) or G2D12 (Right column) compared to whole mucosa and normalized to 18S. A dotted line denotes the expression level in whole mucosa. A representative experiment shows n=3 wells from each condition from a single enteroid line. ATOH1=atonal BHLH transcription factor 1, MUC2=mucin 2, LYZ=lysozyme, ALPI=intestinal alkaline phosphatase, LGR5=leucine-rich repeat-containing G-protein coupled receptor 5, ND=not detectable in one or more samples.

FIG. 1.1B provides a qPCR analysis of chromogranin A (CHGA) expression over time of enteroids grown in G2D12 with Wnt (G2D12+Wnt; circle) or G2D12 without Wnt (G2D12-Wnt; square) compared to whole mucosa and normalized to 18S. RNA was collected every two days after the start of differentiation. The dotted line denotes the expression level in whole mucosa. A representative experiment shows n=3 wells from each condition and timepoint from a single enteroid line. For G2D12+Wnt, only two of three wells expressed CHGA at G2D8 with the non-detectable sample not being included in analysis. ND=not detectable.

FIG. 1.1C illustrates a time course study of total mRNA levels from three enteroid lines (H416 (Left panel), H439 (Center panel), H395 (Right panel)), shown as a percent compared to RNA levels two days after starting experiment (G2), grown in G2D12+Wnt (Circle) or G2D12-Wnt (Square). RNA was collected every two days after start of experiment. A representative experiment shows n=3 wells from each condition from a single enteroid line.

FIG. 1.1D shows a qPCR analysis of intestinal lineage markers of enteroids grown in G2D12 with betacellulin (G2D12+BTC; Right column)) and G2D12 without betacellulin (G2D12-BTC; Center column) compared to enteroids grown in G14 (Left column) and normalized to 18S. A representative experiment shows n=3-5 wells from each condition from a single enteroid line. CHGA=chromogranin A, PDX1=pancreatic and duodenal homeobox 1.

FIG. 1.1E provides a qPCR analysis of intestinal lineage markers of enteroids grown in G2D12 with PF06260933 (G2D12+PF) and G2D12 without PF06260933 (G2D12-PF) compared to enteroids grown in G14 and normalized to 18S. A representative experiment shows n=3-5 wells from each condition from a single enteroid line.

Bars and line graph (FIG. 1.1B) show mean±SEM, *p<0.05, **p<0.01, ***p<0.001, ****p<0.0001. Each experiment repeated with at least three different enteroid lines. Unless otherwise stated, specific conditions were excluded from statistical analysis if the data from one or more samples were labeled as not detectable.

FIG. 2 (FIGS. 2A-2E) illustrates differentiation with small molecules, Rimonabant and SP600125, which induce CHGA expression.

FIG. 2A shows representative light microscopy of enteroids (whole well) grown in growth media for two days followed by 12 days (G2D12) in differentiation media (DM) with rimonabant (Rim) and SP600125 (SP) (RSP). A culture schematic is located above the panel. Scale bar=1 mm.

FIG. 2B provides qPCR analysis of enteroendocrine markers of enteroids grown in G14, G2D12, or RSP compared to whole mucosa and normalized to 18S. Dotted line denotes expression level in whole mucosa. A representative experiment shows n=3 wells from each condition from a single enteroid line. CHGA=chromogranin A, PAX4=paired box 4, PDX1=pancreatic and duodenal homeobox 1, NEUROD1=neuronal differentiation 1, NEUROG3=neurogenin 3, SST=somatostatin, GIP=glucose-dependent insulinotropic peptide, ND=not detectable in one or more samples.

FIG. 2C illustrates representative immunofluorescence staining of CHGA (magenta) in enteroids (whole well) treated with G14 (Left panel), G2D12 (Center panel), and RSP (Right panel). Boxed portion magnified in lower right corner. Nuclei (4′,6-diamidino-2-phenylindole (DAPI), stained blue). RSP and the boxed portion show both CHGA and nuclei stains. Scale bars=1 mm and 50 μm (boxed portion).

FIG. 2D shows the percentage of enteroids with positive CHGA staining in G14, G2D12 and RSP treatments. The table above the graph shows the total number of enteroids examined per condition. Average results are from three separate experiments from three different enteroid lines or passages.

FIG. 2E provides representative flow cytometry plots of CHGA+ cells from enteroids grown in G14, G2D12, or RSP (Left three panels, respectively) and quantification of CHGA+ cells per well (Right panel). A representative experiment shows n=3 wells from each condition from a single enteroid line.

Bars show mean±SEM, ** p<0.01, ***p<0.001, ****p<0.0001. Each experiment repeated with at least three different enteroid lines. Specific conditions were excluded from statistical analysis if the data from one or more samples were labeled as not detectable.

FIG. 2.1 (FIGS. 2.1A-2.1C) demonstrates that a combination of Rimonabant and SP600125 induces enteroendocrine and other intestinal lineage markers.

FIG. 2.1A illustrates a qPCR analysis of enteroendocrine markers of enteroids grown in G2D12 with rimonabant (Rim; Column 3), G2D12 with SP600125 (SP; Column 2), and G2D12 with rimonabant and SP600125 (RSP; Column 4) compared to enteroids grown in G2D12 (Column 1) and normalized to 18S. A representative experiment shows n=3 wells from each condition from a single enteroid line. CHGA=chromogranin A, PDX1=pancreatic and duodenal homeobox 1, NEUROD1=neuronal differentiation 1, NEUROG3=neurogenin 3, SST=somatostatin, GIP=glucose-dependent insulinotropic peptide, GATA4=GATA binding protein 4.

FIG. 2.1B shows a qPCR analysis of intestinal lineage markers of enteroids grown in G14 (Left columns), G2D12 (Center columns), and RSP (Right columns) compared to whole mucosa and normalized to 18S. The dotted line denotes the expression level in whole mucosa. A representative experiment shows n=3 wells from each condition from a single enteroid line. ATOH1=atonal BHLH transcription factor 1, MUC2=mucin 2, LYZ=lysozyme, ALPI=intestinal alkaline phosphatase, LGR5=leucine-rich repeat-containing G-protein coupled receptor 5, ND=not detectable in one or more samples.

Bars show mean SEM, *p<0.05, **p<0.01, ***p<0.001, ****p<0.0001. Each experiment repeated with at least three different enteroid lines. Specific conditions were excluded from statistical analysis if the data from one or more samples were labeled as not detectable.

FIG. 2.1C provides a representative light microscopy of enteroids (whole well) grown in G14 (Left panel), G2D12 (Center panel), and RSP (Right panel). Scale bar=1 mm.

FIG. 3 (FIGS. 3A-3E) illustrates differentiation with small molecule AS1842856, which induces CHGA expression.

FIG. 3A shows representative light microscopy of enteroids (whole well) grown in growth media for two days followed by 12 days in differentiation media (DM) (G2D12) with AS1842856 (AS). A culture schematic is located above the panel. Scale bar=1 mm.

FIG. 3B provides qPCR analysis of enteroendocrine markers of enteroids grown in G14 (Left column), G2D12 (Center column), or AS (Right column) compared to whole mucosa and normalized to 18S. The dotted line denotes the expression level in whole mucosa. A representative experiment showsn=3 wells from each condition from a single enteroid line. CHGA=chromogranin A, PAX4=paired box 4, PDX1=pancreatic and duodenal homeobox 1, NEUROD1=neuronal differentiation 1, NEUROG3=neurogenin 3, CCK=cholecystokinin, SST=somatostatin, GIP=glucose-dependent insulinotropic peptide, ND=not detectable in one or more samples.

FIG. 3C illustrates representative immunofluorescence staining of CHGA (magenta) in enteroids (whole well) treated with G14 (Left panel), G2D12 (Center panel), and AS (Right panel). The boxed portion in the Right panel is magnified in the lower right corner. DNA (4′,6-diamidino-2-phenylindole (DAPI)), is shown in blue primarily in G14 and G2D12, and a combination of CHGA and DNA staining in the Right panel for AS. Scale bars=1 mm and 50 μm (boxed portion).

FIG. 3D provides a percentage of enteroids with positive CHGA staining in G14, G2D12 and AS treatments. The table above the graph shows the total number of enteroids examined per condition. Average results are from three separate experiments from three different enteroid lines or passages.

FIG. 3E shows representative flow cytometry plots of CHGA+ cells from enteroids grown in G14, G2D12 or AS (Left three panels, respectively) and quantification of CHGA+ cells per well (Single panel). A representative experiment shows n=3 wells from each condition from a single enteroid line.

Bars show mean±SEM, *p<0.05, **p<0.01, ****p<0.0001. Each experiment repeated with at least three different enteroid lines. Specific conditions were excluded from statistical analysis if the data from one or more samples were labeled as not detectable.

FIG. 3.1 illustrates AS1842856 (AS) inducing specific intestinal lineage markers.

FIG. 3.1A provides a representative light microscopy of enteroids (whole well) grown in G14 (Left panel), G2D12 (Center panel), and G2D12 with AS1842856 (AS) (Right panel). Scale bar=1 mm.

FIG. 3.1B provides a qPCR analysis of intestinal lineage markers of enteroids grown in G14 (Left column), G2D12 (Center column), and AS (Right column) compared to whole mucosa and normalized to 18S. The dotted line denotes the expression level in whole mucosa. A representative experiment shows n=3 wells from each condition from a single enteroid line. ATOH1=atonal BHLH transcription factor 1, MUC2=mucin 2, LYZ=lysozyme, ALPI=intestinal alkaline phosphatase, LGR5=leucine-rich repeat-containing G-protein coupled receptor 5.

Bars show mean±SEM, *p<0.05, ****p<0.0001. Each experiment repeated with at least three different enteroid lines.

FIG. 4 demonstrates scRNA seq profiling of enteroids cultured with Rimonabant/SP600125 or AS1842856.

FIG. 4A shows Uniform Manifold Approximation and Projection (UMAP) visualization of 14,767 cells summarizing enteroid differentiation from all samples, color labeled by culture condition. Differentiation Media (DM), Rimonabant/SP600125 (RSP), and AS1842856 (AS).

FIG. 4B illustrates UMAP visualization from FIG. 4A, color labeled by broad annotated cell identity, following Louvain clustering.

FIG. 4C provides a dot plot of the average scaled expression (measured by average Pearson residual) of canonical markers of various gut epithelial cell types, plotted against cluster identity. The average expression of two exhibited in Intestinal Stem Cells (ISCs) for ASCL2, AXIN2, and LGR5; Proliferating Progenitor Cells (PPCs) for MKI67; Enterocytes for KRT20 and FABP1; Goblet cells for GFI1, FCGBP, and MUC2; Enteroendocrine cells (EeCs) for PAX4, NEUROD1, NEUROG3, and CHGA.

FIG. 4D demonstrates proportional abundance of epithelial cell subsets by enteroid culture protocol: G2D12 (Left column), RSP (Center column), AS (Right column) for each cell type. Each culture condition consists of three different enteroid lines from distinct human donors, as denoted by data point shape: H357 (Circle), H389 (Square), H407 (Triangle). Bars show mean±SEM, *p<0.05. Statistical significance was calculated using the Kruskal-Wallis test with Dunn's post-hoc analysis being displayed. p values were adjusted for multiple hypothesis testing using the Bonferroni correction.

FIG. 4E shows UMAP visualization of 14,767 cells divided by culture condition: G2D12 (left); RSP (right); AS (below) and colored by cell identity. Intestinal Stem Cells (ISC); Proliferating Progenitor Cells (PPC); Progenitor Cells (PC); Enterocytes; Goblet Cells (Goblet); and Enteroendocrine Cells (EeC). Trajectory analysis of each protocol was calculated using scVelo, and the vector field was overlaid on top of each UMAP. Arrows represent smoothed averages of the estimated cellular differentiation trajectory, with arrow thickness corresponding to the “speed” of differentiation.

FIG. 4F illustrates UMAP visualization from FIG. 4A, with individual cells colored by their Enteroendocrine (EE) cell module score. EE cell module scores were derived and calculated from genes enriched in in vivo EE cells isolated from the murine small intestine. Each score was scaled on a range from 0 to 1.

FIG. 4G provides violin plots of the module score described in FIG. 4F for all cell types, split across culture condition (Left to right: G2D12; RSP; AS). The effect size between culture conditions was calculated as Cohen's d; $$0.8<d<1.2.

FIG. 4.1 (FIGS. 4.1A-4.1D) demonstrates cellular validation and characterization of enteroid single cell transcriptomes.

FIG. 4.1A shows violin plots summarizing the distribution of genes (left), unique molecular identifiers (center), and the proportion of mitochondrial genes (right) within the scRNA-seq dataset.

FIG. 4.1B illustrates tSNE visualization of 23,335 cells from all samples with color denoting the hashtag identity. Singlet cells were denoted as having significant enrichment of only one hashtag signal per individual cell. Doublet cells had significant enrichment of more than one hashtag signal.

FIG. 4.1C provides UMAP visualization of 25,673 cells originating from all samples, with color being used to distinguish between negative, doublet, or singlet cells, as described in FIG. 4.1B.

FIG. 4.1D demonstrates UMAP visualization from FIG. 4.1C with cells labeled by their Louvain cluster identity.

FIG. 5 illustrates combinations of AS1842856 (AS) and Rimonabant/SP600125 (RSP) that induce different levels of enteroendocrine marker expression.

FIG. 5A provides qPCR analysis of enteroendocrine markers of enteroids grown in AS, AS for 6 days, followed by RSP only for 6 days (AS→RSP) and AS for 6 days, followed by AS and RSP for 6 days (AS→RASP) compared to enteroids grown in RSP and normalized to 18S. A representative experiment shows n=3 wells from each condition from a single enteroid line. Top panel: CHGA=chromogranin A, PAX4=paired box 4, PDX1=pancreatic and duodenal homeobox 1, NEUROD1=neuronal differentiation 1, NEUROG3=neurogenin 3, Bottom panel: CCK=cholecystokinin, SST=somatostatin, GIP=glucose-dependent insulinotropic peptide. Columns from left to right: RSP; AS; AS→RSP; and AS→RASP, respectively.

FIG. 5B shows representative immunofluorescence staining of CHGA (magenta) in enteroids (whole well) treated with G14, G2D12, AS, RSP, AS→RSP, and AS→RASP. DNA (4′,6-diamidino-2-phenylindole (DAPI)) shown in blue, primarily expressed in G14, G2D12, and RSP (Top row) and in the background of AS, AS→RSP, and AS→RASP (Bottom row). CHGA expression displayed as magenta seen slightly in RSP, more in AS, less in AS→RSP, and more in AS→RASP. Scale bar=1 mm.

FIG. 5C illustrates percentage of enteroids with positive CHGA staining (Y-axis) in G14, G2D12, RSP, AS, AS→RSP, and AS→RASP treatments, respectively from Left to Right (X-axis). The table above the graph shows the total number of enteroids examined per condition. Average results are from three separate experiments from three different enteroid lines or passages.

FIG. 5D provides representative flow cytometry plots of CHGA+ cells from enteroids grown in G14, G2D12, AS, RSP, AS to RSP, and AS to RASP (Six panels) and quantification of CHGA+ cells per well (Single panel). A representative experiment shows n=3 wells from each condition from a single enteroid line.

Bars show mean±SEM, *p<0.05, **p<0.01, ***p<0.001, ****p<0.0001. Each experiment repeated with at least three different enteroid lines.

FIG. 5.1 (FIGS. 5.1A-5.1C) demonstrates that the combination of AS1842856 and Rimonabant/SP600125 for all of the differentiation leads to reduction in isolated RNA.

FIG. 5.1A shows a qPCR analysis of enteroendocrine markers of enteroids grown in AS (Right column) compared to RSP (Left column) and normalized to 18S. A representative experiment shows n=3 wells from each condition from a single enteroid line. CHGA=chromogranin A, PDX1=pancreatic and duodenal homeobox 1, NEUROD1=neuronal differentiation 1, NEUROG3=neurogenin 3, SST=somatostatin, GIP=glucose-dependent insulinotropic peptide.

FIG. 5.1B provides total mRNA levels from enteroids grown in G14, G2D12, AS, RSP, and RASP. Representative results from n=2-3 wells from each condition from a single enteroid line. Bars show mean±SEM, *p<0.05, **p<0.01, ***p<0.001, ****p<0.0001. Each experiment repeated with at least three different enteroid lines.

FIG. 5.1C demonstrates a representative light microscopy of enteroids (whole well) grown in Top row: G14 (Left panel), G2D12 (Center panel), RSP (Right panel); Bottom row: AS (Left panel) and G2D12 with AS and RSP (RASP) (Right panel). Scale bar=1 mm.

FIG. 6 shows multiple differentiation conditions that induce hormone production.

FIG. 6A illustrates representative immunofluorescence staining of somatostatin (SST, green) and chromogranin A (CHGA, magenta) in enteroids treated with G14, G2D12, RSP, AS, AS→RSP, and AS→RASP, respectively from Left to Right. DNA (4′,6-diamidino-2-phenylindole (DAPI)) is stained in blue. Scale bar=50 μm.

FIG. 6B provides a percentage of enteroids with positive SST staining in G14, G2D12, RSP, AS, AS→RSP, and AS→RASP treatments, respectively from Left to Right. The table above the graph shows the total number of enteroids examined per condition.

FIG. 6C shows representative immunofluorescence staining of serotonin (5HT, green) and chromogranin A (CHGA, magenta) in enteroids treated with G14, G2D12, RSP, AS, AS→RSP and AS→RASP, respectively from Left to Right. DNA (4′,6-diamidino-2-phenylindole (DAPI)), shown in blue. Scale bar=50 μm.

FIG. 6D demonstrates percentage of enteroids with positive 5HT staining in G14, G2D12, RSP, AS, AS→RSP, and AS→RASP treatments, respectively from Left to Right. The table above the graph shows the total number of enteroids examined per condition.

FIG. 6E provides representative immunofluorescence staining of glucose-dependent insulinotropic peptide (GIP, green) and chromogranin A (CHGA, magenta) in enteroids treated with G14, G2D12, RSP, AS, AS→RSP, and AS→RASP, respectively from Left to Right. DNA (4′,6-diamidino-2-phenylindole (DAPI), shown in blue blue). Scale bar=50 μm.

FIG. 6F shows a percentage of enteroids with positive GIP staining in G14, G2D12, RSP, AS, AS→RSP, and AS→RASP treatments, respectively from Left to Right.

FIG. 6G illustrates representative immunofluorescence staining of cholecystokinin (CCK, green) and CHGA (magenta) in enteroids treated with G14, G2D12, RSP, AS, AS→RSP, and AS→RASP. DNA stained blue (DAPI). Scale bar=50 μm.

FIG. 6H demonstrates percentage of enteroids with positive CCK staining in G14, G2D12, RSP, AS, AS→RSP, and AS→RASP treatments, respectively from Left to Right. The table above the graph shows the total number of enteroids examined per condition. Bars show mean±SEM, *p<0.05, **p<0.01, ***p<0.001, ****p<0.0001. Tables above the graphs show the total number of enteroids examined per condition. Average results are from three separate experiments from three different enteroid lines or passages. Each experiment repeated with at least three different enteroid lines.

FIG. 6.1 (FIGS. 6.1A-6.1D) illustrates the combination of AS1842856 and Rimonabant/SP600125 after initial AS1842856 exposure yields viable enteroids.

FIG. 6.1A provides a qPCR analysis of enteroendocrine marker expression over time from enteroids grown in AS compared to whole mucosa and normalized to 18S. RNA was collected every two days after start of differentiation. The dotted line denotes the expression level in whole mucosa. A representative experiment shows n=3 wells from each timepoint, except n=2 for G2D12, from a single enteroid line. At G2D2, only two of three wells expressed NEUROD1 and NEUROG3, with nondetectable samples excluded from analysis. CHGA=chromogranin A, NEUROD1=neuronal differentiation 1, NEUROG3=neurogenin 3, SST=somatostatin, ND=not detectable.

FIG. 6.1B shows total mRNA levels from enteroids grown in RSP, AS, AS→RSP, and AS→RASP, respectively from Left to Right columns. A representative experiment shows n=3 wells from each condition from a single enteroid line. Bars and line graph show mean±SEM, *p<0.05, **p<0.01. Each experiment repeated with at least three different enteroid lines.

FIG. 6.1C provides a representative light microscopy of enteroids (whole well) grown in Top row: G14 (Left panel), G2D12 (Center panel), RSP (Right panel); Bottom row: AS (Left panel), AS→RSP (Center panel), and AS→RASP (Right panel), respectively from Left to Right. Specific culture schematics of AS→RSP, and AS→RASP are located above each panel, respectively. Scale bar=1 mm.

Bars and line graph show mean±SEM, *p<0.05, **p<0.01, ***p<0.001, ****p<0.0001. Each experiment repeated with at least three different enteroid lines.

FIG. 7 illustrates differentiation conditions that induce hormone secretion.

FIG. 7A provides a serotonin (5HT) ELISA of conditioned media from the last two days of differentiation of enteroids grown in G14, G2D12, RSP, AS, AS→RSP, and AS→RASP, from Left to Right, respectively. The dotted line at 2.6 ng/mL represents the lower limit of detection for the assay. A representative experiment shows n=3 wells from each condition from a single enteroid line.

FIG. 7B demonstrates a 5HT ELISA of AS conditioned media collected after 24 hours on day 13 (left bar) and after 24 hours with forskolin (Fsk) on day 14 (right bar). The dotted line at 2.6 ng/mL represents the lower limit of detection for the assay. A representative experiment shows n=3 wells from each condition from a single enteroid line.

FIG. 7C shows a glucose-dependent insulinotropic peptide (GIP) ELISA of conditioned media from the last two days of differentiation of enteroids grown in G14, G2D12, AS, RSP, AS→RSP, and AS→RASP. The dotted line at 4.2 pg/mL represents the lower limit of detection for the assay. A representative experiment shows n=3 wells from each condition from a single enteroid line.

FIG. 7D illustrates a GIP ELISA of AS→RSP conditioned media collected after 24 hours on day 13 (left bar) and after 24 hours with Fsk on day 14 (right bar). The dotted line at 4.2 pg/mL represents the lower limit of detection for the assay. A representative experiment shows n=3 wells from each condition from a single enteroid line.

Bars show mean±SEM, *p<0.05, **p<0.01, ***p<0.001, ****p<0.0001. Each experiment repeated with at least three different enteroid lines.

FIG. 7.1 illustrates induction of specific intestinal lineage markers, cell viability, and proliferation compared across differentiation methods.

FIG. 7.1A shows qPCR analysis of intestinal lineage markers of enteroids grown in AS (Column 2), AS→RSP (Column 3), and AS→RASP (Column 4) compared to RSP (Column 1) and normalized to 18S. A representative experiment showing n=3 wells from each condition from single enteroid line. ATOH1=atonal BHLH transcription factor 1; MUC2=mucin 2; LYZ=lysozyme; ALPI=intestinal alkaline phosphatase; LGR5=leucine-rich repeat-containing G-protein coupled receptor 5.

FIG. 7.1B demonstrates representative immunofluorescence staining of cytokeratin 20 (CK20, green) in enteroids treated with G14, G2D12, RSP, AS, AS→RSP, and AS→RASP. DNA (4′,6-diamidino-2-phenylindole (DAPI), in blue). Scale bar=50 μm. CK20 primarily expressed in G2D12 and RSP, and to lesser degrees AS, AS→RSP, and AS→RASP. DNA expressed throughout all culture conditions.

FIG. 7.1C provides representative immunofluorescence staining of mucin 2 (MUC2, green) in enteroids treated with G14, G2D12, RSP, AS, AS→RSP, and AS→RASP. DNA (4′,6-diamidino-2-phenylindole (DAPI), shown in blue). Scale bar=50 μm. MUC2 primarily expressed in RSP and AS→RSP, and to a lesser degree AS, while not observable in G14, G2D12, and AS→RASP. DNA expressed throughout all culture conditions.

FIG. 7.1D illustrates representative immunofluorescence staining of lysozyme (LYZ, green) in enteroids treated with G14, G2D12, RSP, AS, AS→RSP, and AS→RASP. DNA (4′,6-diamidino-2-phenylindole (DAPI), in blue). Scale bar=50 μm. LYZ expressed in G2D12, RSP, AS, and AS→RSP, while DNA was expressed throughout all culture conditions.

FIG. 7.1E shows percentage of EdU-positive cells after a two-hour chase with 10 μM EdU in enteroids grown in their respective medias at either 7 days (left side) or 14 days (right side). G7/G14 (Column 1); G2D5/G2D12 (Column 2); RSP (Column 3); AS (Column 4); AS→RSP (Column 5), and AS→RASP (Column 6).

FIG. 7.1F demonstrates percentage of Annexin V-positive cells in enteroids grown in their respective medias at either 7 days (left side) or 14 days (right side). G7/G14 (Column 1); G2D5/G2D12 (Column 2); RSP (Column 3); AS (Column 4); AS→RSP (Column 5), and AS→RASP (Column 6).

Bars and line graph show mean±SEM, *p<0.05, ***p<0.001, ****p<0.0001. Each experiment repeated with at least three different enteroid lines.

FIG. 8 illustrates induction of the enteroendocrine lineage in rectoids.

FIG. 8A provides a representative light microscopy of rectoids (whole well) grown in growth media (GM) for 14 days (G14) or two days in GM followed by 12 days in differentiation media (DM, G2D12). Scale bar=1 mm.

FIG. 8B presents qPCR analysis of enteroendocrine (EE) markers of rectoids grown in either G14 (Left column) or G2D12 (Right column) compared to whole mucosa and normalized to 18S. The dotted line denotes the expression level in whole mucosa. A representative experiment shows n=3 wells for each condition from a single enteroid line. CHGA=chromogranin A, PAX4=paired box 4, NEUROD1=neuronal differentiation 1, NEUROG3=neurogenin 3, SST=somatostatin, GCG=glucagon, PYY=peptide YY, ND=not detectable in 1 or more samples.

FIG. 8C shows representative immunofluorescence staining of CHGA (magenta) in rectoids (whole well) treated with G14 and G2D12. DNA (4′,6-diamidino-2-phenylindole (DAPI)) shown in blue. Scale bar=1 mm. G14 primarily expressed DNA staining, while G2D12 had a mixture of CHGA and DNA staining.

FIG. 8D illustrates the percentage of rectoids with positive CHGA staining in G14 and G2D12 treatments. The table above the graph shows the total number of rectoids examined per condition. The average results are from three separate experiments from three different enteroid lines or passages.

FIG. 8E provides representative flow cytometry plots of CHGA+ cells from rectoids grown in G14 and G2D12 (left two panels) based on their side scatter area (SSC-A). Quantification of CHGA+ cells (% cells CHGA+) per well is shown in the right panel. A representative experiment shows n=3 wells from each condition from a single enteroid line.

FIG. 8F demonstrates representative immunofluorescence staining of glucagon-like peptide-1 (GLP-1, green) and CHGA (magenta) in rectoids treated with G14 and G2D12. DNA (DAPI) shown in blue. Scale bar=50 μm. CHGA and GLP-1 staining expressed in G2D12, and DNA observed in G14 and G2D12.

FIG. 8G shows percentage of rectoids with positive GLP-1 staining in G14 and G2D12. The table above the graph shows the total number of rectoids examined per condition. Average results are from three separate experiments from three different enteroid lines or passages.

FIG. 8H illustrates representative immunofluorescence staining of peptide YY (PYY, green) and CHGA (magenta) in rectoids treated with G14 and G2D12. DNA (DAPI) shown in blue. Scale bar=50 μm. CHGA and PYY staining expressed in G2D12, and DNA observed in G14 and G2D12.

FIG. 8I provides percentage of rectoids with positive PYY staining in G14 and G2D12. The table above the graph shows the total number of rectoids examined per condition. Average results are from three separate experiments from three different enteroid lines or passages.

FIG. 8J presents GLP-1 ELISA of conditioned media from the last two days of differentiation of rectoids grown in G14 and G2D12. The dotted line at 25 pg/mL represents the lower limit of detection for the assay. A representative experiment shows n=3 wells from each condition from a single rectoid line.

FIG. 8K shows GLP-1 ELISA of G2D12 conditioned media collected after 24 hours on G2D12 (left bar) and after 24 hours with forskolin (Fsk) on G2D12 (right bar). The dotted line at 25 pg/mL represents the lower limit of detection for the assay. A representative experiment shows n=3 wells from each condition from a single rectoid line.

FIG. 8L illustrates PYY ELISA of conditioned media from the last two days of differentiation of rectoids grown in G14 and G2D12. The dotted line at 7.3 pg/mL represents the lower limit of detection for the assay. A representative experiment shows n=3 wells from each condition from a single rectoid line.

FIG. 8M presents PYY ELISA of G2D12 conditioned media collected after 24 hours on G2D1 (left bar) and after 24 hours with forskolin (Fsk) on G2D12 (right bar). The dotted line at 7.3 pg/mL represents the lower limit of detection for the assay. A representative experiment shows n=3 wells from each condition from a single rectoid line.

Bars show mean±SEM, *p<0.05, **p<0.01, ***p<0.001, ****p<0.0001. Each experiment repeated with at least three different rectoid lines. Specific conditions were excluded from statistical analysis if the data from one or more samples were labeled as not detectable.

FIG. 8.1 demonstrates hormone secretion of different conditions controlled for total DNA.

FIG. 8.1A provides serotonin (5HT) ELISA of conditioned media from the last two days of differentiation of enteroids grown in G14, G2D12, RSP, AS, AS→RSP, and AS→RASP, respectively from Left to Right, controlled for total DNA from each sample. A representative experiment shows n=3 wells from each condition from a single enteroid line.

FIG. 8.1B shows 5HT ELISA of AS conditioned media collected after 24 hours on day 13 (left bar) and after 24 hours with forskolin (Fsk) on day 14 (right bar) controlled for total DNA from each sample. A representative experiment shows n=3 wells from each condition from a single enteroid line.

FIG. 8.1C illustrates Glucose-dependent insulinotropic peptide (GIP) ELISA of conditioned media from the last two days of differentiation of enteroids grown in G14, G2D12, RSP, AS, AS→RSP, and AS→RASP, respectively from Left to Right) controlled for total DNA from each sample. A representative experiment shows n=3 wells from each condition from a single enteroid line.

FIG. 8.1D presents GIP ELISA of AS→RSP conditioned media collected after 24 hours on day 13 (left bar) and after 24 hours with Fsk on day 14 (right bar) controlled for total DNA from each sample. A representative experiment shows n=3 wells from each condition from a single enteroid line.

Bars show mean±SEM, *p<0.05, **p<0.01, ***p<0.001, ****p<0.0001. To control for enteroid number, protein concentrations were divided by DNA concentration. Each experiment repeated with at least three different enteroid lines

FIG. 9 demonstrates induction of other lineage markers in rectoids and rectoid hormone secretion controlled for total DNA.

FIG. 9A provides qPCR analysis of intestinal lineage markers of rectoids grown in G14 (Left column) and G2D12 (Right column) compared to whole mucosa and normalized to 18S. The dotted line denotes the expression level in whole mucosa. A representative experiment shows n=3 wells from each condition from a single rectoid line. ATOH1=atonal BHLH transcription factor 1, MUC2=mucin 2, CAII=carbonic anhydrase II, LGR5=leucine-rich repeat-containing G-protein coupled receptor 5.

FIG. 9B shows Glucagon-like peptide-1 (GLP-1) ELISA of conditioned media from the last two days of differentiation of enteroids grown in G14 and G2D12 controlled for total DNA from each sample. A representative experiment shows n=3 wells from each condition from a single rectoid line.

FIG. 9C illustrates GLP-1 ELISA of G2D12 conditioned media collected after 24 hours on day 13 (left bar) and after 24 hours with forskolin (Fsk) on day 14 (right bar) controlled for total DNA from each sample. A representative experiment shows n=3 wells from each condition from a single rectoid line.

FIG. 9D demonstrates Peptide YY (PYY) ELISA of conditioned media from the last two days of differentiation of enteroids grown in G14 and G2D12 controlled for total DNA from each sample. A representative experiment shows n=3 wells from each condition from a single rectoid line.

FIG. 9E provides PYY ELISA of G2D12 conditioned media collected after 24 hours on day 13 (left bar) and after 24 hours with Fsk on day 14 (right bar) controlled for total DNA from each sample. A representative experiment shows n=3 wells from each condition from a single enteroid line.

Bars show mean±SEM, **p<0.01, ***p<0.001, ****p<0.0001. To control for enteroid number, protein concentrations were divided by DNA concentration. Each experiment repeated with at least three different enteroid lines.

FIG. 10 illustrates a gating strategy for flow cytometry.

FIG. 10A presents enteroid cells that are differentiated from cellular debris based on their forward and side scatter areas (FSC-A and SSC-A, respectively) parameters. Cells are then examined based on their FSC-A and FSC-Height (H) to exclude doublets. 4′,6-diamidino-2-phenylindole (DAPI) staining is then utilized to identify dead cells, with DAPI high-positive cells being excluded from further gating.

FIG. 10B demonstrates the CHGA-positive gate set by using an IgG1 K isotype control conjugate with phycoerythrin (PE).

FIG. 10C provides the CHGA-positive gate set by using an IgG2b K isotype control conjugate with Alexa fluor 647 (APC).

### DETAILED DESCRIPTION OF THE INVENTION

Discovery of new and improved compositions and methods of producing and using enteroendocrine (EE) cells may enable drug development and ultimately lead to advances in clinical care. Enteroendocrine (EE) cells are the most abundant hormone-producing cells in humans and are critical regulators of energy homeostasis and gastrointestinal function. Non-limiting examples of EE hormones include: ghrelin, gastrin, histamine, leptin (from stomach), secretin, GIP, CCK, SST, serotonin, motilin, neurotensin, GLP-1, GLP-2, PYY, and oxyntomodulin. Some embodiments of the disclosure are directed to an in vitro generated three-dimensional gastrointestinal enteroid/organoid derived from an induced human gastrointestinal stem cell (GISC), the enteroid/organoid comprising functional human enteroendocrine cells, where the enteroendocrine cells secrete hormones, such as but not limited to, ghrelin, gastrin, histamine, leptin (from stomach), secretin, GIP, CCK, SST, serotonin, motilin, neurotensin, GLP-1, GLP-2, PYY, and oxyntomodulin. Another embodiment may be directed to such organoids comprising EE cells that secrete hormones selected from: GIP, CCK, SST, serotonin, GLP-1, PYY, and combinations thereof. Enteroendocrine cells, and the hormones they secrete, play critical roles in normal human physiology, including overall gastrointestinal function and appetite regulation, as well as in the pathophysiology of diseases affecting as many as 70 million Americans, such as Type II diabetes, irritable bowel syndrome, and inflammatory bowel disease. Embodiments of the invention feature gastrointestinal stem cell (GISC) derived organoids or enteroids comprising enteroendocrine cells useful as an in vitro or ex vivo model to study genetic, molecular, and cellular abnormalities associated with, for example, human diseases. This organoid recapitulates in vitro or ex vivo the development, physiology, and other characteristics of the gastrointestinal (GI) tract (e.g., mammal, human, rodent). The invention, in some embodiments, further provides methods of using this gastrointestinal organoid or enteroid to study disease and to identify therapeutic agents for the treatment of metabolic and/or gastrointestinal diseases and symptoms thereof.

EE cell dysfunction is found in many metabolic and gastrointestinal diseases and conditions, including but not limited to obesity, diabetes, post-infectious irritable bowel syndrome, infectious enteritis, and inflammatory bowel disease. Featured herein are methods and systems for the generation and utilization of stem cell-derived human enteroendocrine cell organoids, which provide a promising strategy for the therapeutic treatment of diseases and pathologies, such as metabolic and gastrointestinal diseases, such as but not limited to, obesity, type II diabetes mellitus, irritable bowel syndrome (IBS), inflammatory bowel disease (IBD), Crohn's disease, and ulcerative colitis. Advantageously, the methods and systems as described here can generate biological products, e.g., cells, human enteroendocrine cell organoids derived from human gastrointestinal stem cells (GISCs) and enteroendocrine (EE) cells thereof, as therapeutics that can be used to treat patients having an EE cell dysfunction.

The invention is based, at least in part on methods useful for engineering a human gastrointestinal organoid that after −2 weeks of culture in vitro exhibits a level of development comparable to that of a functioning human GI system, since the epithelial cells, such as enteroendocrine cells, are continuously renewed, replenished, and/or repaired. These organoids express markers characteristic of a large variety of enteroendocrine cells. This organoid is useful as a platform to enable screening of agents for efficacy, safety, and toxicity prior to in vivo use in humans.

In particular embodiments, organoids are derived from undifferentiated gastrointestinal stem cells that result in daughter or progenitor cells, which subsequently differentiate into enteroendocrine cells required for normal GI function. These organoids may be formed on plates, e.g., 96-well plates. The organoid model is under development to reach an FDA metric for clinical diagnostic use and drug development.

### Targets and Signaling Pathways in Enteroendocrine Cell Formation and Function

The GATA Binding Protein 4 (GATA4) transcription factor was found to be essential for overall gut formation and plays an important role in specifying enteroendocrine cell identity, including glucose-dependent insulinotropic polypeptide (GIP)-expressing cells (Jepeal L I, Boylan M O, Michael Wolfe M. Mol Cell Endocrinol 2008; 287:20-9; Walker E M, Thompson C A, Battle M A. Dev Biol 2014; 392:283-94). c-Jun N-terminal kinase (JNK) has been shown to play a role in enteroendocrine cell differentiation. Inhibition of JNK signaling, which has been implicated in regulating ISCs (Biteau B, Hochmuth C E, Jasper H. Cell Stem Cell 2008; 3:442-55), also regulates endocrine cells through its actions on pancreatic and duodenal homeobox 1 (PDX1) (Kaneto H, Matsuoka T A, Nakatani Y, et al. Curr Diabetes Rev 2005; 1:65-72; Tang C, Yeung L S N, Koulajian K, et al. Endocrinology 2018; 159:3643-3654) expression, which is increased. JNK may exert its effect on PDX1 through a post-transcriptional mechanism. For example, in murine islet cells, JNK has been associated with increased nucleocytoplasmic translocation of PDX1, which indirectly affects the transcriptional function of PDX1 without affecting its mRNA level (Kaneto H, Matsuoka T A, Nakatani Y, et al. Curr Diabetes Rev 2005; 1:65-72). It is also possible that JNK inhibition does not act on PDX1 (mRNA or protein) at all in human gastrointestinal cells; its role in human intestinal stem cell differentiation has not been well-studied. However, JNK inhibition does appear to have an effect on mRNA expression levels of specific EE markers. FOXO1 has been implicated in beta cell differentiation, affecting the expression of NEUROG3 and NEUROD1, as well as multiple other transcription factors (Bouchi R, Foo K S, Hua H, et al. Nat Commun 2014; 5:4242; Glauser D A, Schlegel W. J Endocrinol 2007; 193:195-207; Talchai C, Xuan S, Kitamura T, et al. Nat Genet 2012; 44:406-12, S1). Several studies have shown that FOXO1 inhibition can induce differentiation of insulin-producing cells from gastrointestinal cells, both in vivo and in enteroid cultures (Bouchi R, Foo K S, Hua H, et al. Nat Commun 2014; 5:4242; Talchai C, Xuan S, Kitamura T, et al. Nat Genet 2012; 44:406-12, S1); however, these studies did not closely examine EE cell populations. Inhibition of forkhead box protein 01 (FOXO1), a transcription factor critical for stem cell function and energy homeostasis (Kousteni S. Bone 2012; 50:437-43; Tothova Z, Gilliland D G. Cell Stem Cell 2007; 1:140-52), has been associated with upregulation of multiple endocrine-associated transcription factors and hormones, including neurogenin 3 (NEUROG3) and GIP (Roy S A, Langlois M J, Carrier J C, et al. World J Gastroenterol 2012; 18:1579-89; Garcia-Martinez J M, Chocarro-Calvo A, De la Vieja A, et al. Biochim Biophys Acta 2014; 1839:1141-50; Bouchi R, Foo K S, Hua H, et al. Nat Commun 2014; 5:4242; Arriola D J, Mayo S L, Skarra D V, et al. J Biol Chem 2012; 287:33424-35). Finally, bone morphogenic protein 4 (BMP4) has been shown to induce the expression of various EE hormones in human intestinal enteroids (Beumer J, Artegiani B, Post Y, et al. Nat Cell Biol 2018; 20:909-916).

GATA4 activation/JNK inhibition and FOXO1 inhibition have been found to increase expression of the same genes involved in EE differentiation. Further, GATA4, JNK, and FOXO1 have not been identified in large scale screens or single cell sequencing studies examining the EE lineage (Fujii M, Matano M, Toshimitsu K, et al. Cell Stem Cell 2018; 23:787-793 e6; Beumer J, Artegiani B, Post Y, et al. Nat Cell Biol 2018; 20:909-916), including two single cell RNA sequencing studies analyzing human and munne EE lineage differentiation using neurogenin 3 reporters (Gehart H, van Es J H, Hamer K, et al. Cell 2019; 176:1158-1173 e16; Beumer J, Puschhof J, Bauza-Martinez J, et al. Cell 2020). This suggests that GATA4, JNK, and FOXO1 may work upstream of neurogenin 3.

Exposure of enteroids to combinations of GATA4 activation, JNK inhibition, and FOXO1 inhibition lead to the expression of specific hormones, with GATA4 activation/JNK inhibition inducing glucose-dependent insulinotropic polypeptide (GIP) expression and FOXO1 inhibition inducing higher levels of serotonin (5HT) expression. Exposure to FOXO1 inhibitor, AS1842856 (AS)→putative GATA4 activator or CB1 inverse agonist, Rimonabant (Rim) and JNK inhibitor, SP600125 (SP) (RSP) yielded similar levels of GIP mRNA and total GIP+ enteroids, but increased levels of GIP secretion, when compared to RSP alone; however, AS→Rim, AS, and SP (RASP) seemingly inhibited GIP protein production and secretion. FOXO1 inhibition alone appears to be the most potent inducer of 5HT secretion. Exposure to RSP, with or without AS, induced less 5HT secretion when compared to FOXO1 inhibition alone. Taken together, the combinatorial data described here suggest that FOXO1 inhibition is a potent inducer of 5HT secretion while inhibiting GIP secretion, and GATA4 activation/JNK inhibition is a potent inducer of GIP secretion while leading to reduced 5HT secretion when compared to AS-treated enteroids. The data of the disclosure also suggest that modulation of GATA4, JNK, and FOXO1 have multiple effects on EE function, controlling mRNA and protein production, as well as secretion, of multiple hormones, for example, cholecystokinin (CCK), glucagon-like peptide-1 (GLP-1), glucose-dependent insulinotropic polypeptide (GIP), peptide YY (PYY), serotonin (5HT), and somatostatin (SST). Other embodiments may be directed to EE secreted hormones, such as but not limited to, cholecystokinin (CCK), gastrin, ghrelin, glucagon-like peptide-1 (GLP-1), GLP-2, glucose-dependent insulinotropic polypeptide (GIP), histamine, leptin, motilin, neurotensin, oxyntomodulin, peptide YY (PYY), secretin, serotonin (5HT), and somatostatin (SST).

Some embodiments of the disclosure, as described herein, provide methods for inducing differentiation of human GISCs into enteroendocrine cells using only small molecules, such as but not limited to, putative activators of GATA Binding Protein 4 (GATA4) or CB1 inverse agonist (e.g., rimonabant (Rim)) and inhibitors of c-Jun N-terminal kinase (JNK) or pancreatic and duodenal homeobox 1 (PDX1) (e.g., SP600125 (SP)) and of forkhead box protein 01 (FOXO1) (e.g., AS1842856 (AS), which are key transcriptional regulators known to mediate endodermal development and hormone production. Briefly, human GISCs may be grown ex vivo in three-dimensional (3D) cultures or organoids. This enables the maintenance and differentiation of GISCs in a controlled environment to better study differentiated cells and their role in gastrointestinal (GI) physiology and disease. EE cells, and their hormones they produce, are becoming increasingly critical in GI function and pathophysiology, such that there is a desire to improve present methods of growing enteroendocrine cells ex vivo and in vitro, to obtain a better understanding of their differentiation and/or function, and their potential as therapeutics.

Enteroendocrine cells, as a class, are defined by expression of the neuroendocrine secretory protein Chromogranin A (CHGA), the specific hormone each produces, and their location along the GI tract (Furness J B, Rivera L R, Cho H J, et al. Nat Rev Gastroenterol Hepatol 2013; 10:729-40; Sinagoga K L, McCauley H A, Munera J O, et al. Development 2018; 145; Gehart H, van Es J H, Hamer K, et al. Cell 2019; 176:1158-1173 e16; Beumer J, Artegiani B, Post Y, et al. Nat Cell Biol 2018; 20:909-916). Further, multiple transcription factors are critical for EE cell differentiation and function, including, but not limited to neurogenin 3 (NEUROG3), neuronal differentiation 1 (NEUROD1), and pancreatic and duodenal homeobox 1 (PDX1) (Posovszky C. Endocr Dev 2017; 32:20-37; Furness J B, Rivera L R, Cho H J, et al. Nat Rev Gastroenterol Hepatol 2013; 10:729-40; Basak O, Beumer J, Wiebrands K, et al. Cell Stem Cell 2017; 20:177-190 e4; Chen C, Fang R, Davis C, et al. Am JPhysiol Gastrointest Liver Physiol 2009; 297:G1126-37).

### Screening Assays

Gastrointestinal organoids may be used for toxicity and efficacy screening of agents that treat or prevent the development of a metabolic and/or gastrointestinal disease having an enteroendocrine cell dysfunction. In one embodiment, an organoid generated according to the methods described herein is contacted with a candidate agent. The viability of the organoid (or various enteroendocrine cells within the organoid) is compared to the viability of an untreated control organoid to characterize the toxicity of the candidate compound. Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 0.1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J., Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

In another embodiment, the organoid comprises a genetic mutation that affects differentiation, development, activity, or function of enteroendocrine cells. Polypeptide or polynucleotide expression of cells within the organoid can be compared by procedures well known in the art, such as Western blotting, flow cytometry, immunocytochemistry, in situ hybridization, fluorescence in situ hybridization (FISH), ELISA, microarray analysis, RT-PCR, Northern blotting, or colorimetric assays, such as the Bradford Assay and Lowry Assay.

A further embodiment provides for a method comparing gene expression levels of endocrine cell markers between differentiated organoids or enteroids from the duodenum and freshly isolated whole mucosa from human duodenal biopsies, which serve as a proxy for native in situ gene expression levels.

In a working example, one or more candidate agents may be added at varying concentrations to the culture medium containing an organoid. An agent that promotes the expression of a polypeptide of interest expressed in the cell is considered useful in the invention; such an agent may be used, for example, as a therapeutic to prevent, delay, ameliorate, stabilize, or treat an injury or disease characterized by a defect in enteroendocrine cell differentiation, development, or function. Once identified, agents of the invention may be used to treat or prevent a metabolic and/or gastrointestinal disease that has, for example, an enteroendocrine cell dysfunction.

In another embodiment, the activity or function of a cell of the organoid is compared in the presence and the absence of a candidate compound. Compounds that desirably alter the activity or function of the cell are selected as useful in the methods of the invention.

### Test Compounds and Extracts

In general, agents useful in the invention are identified from large libraries of natural product or synthetic (or semi-synthetic) extracts or chemical libraries or from polypeptide or nucleic acid libraries, including primer sets, according to methods known in the art. Those skilled in the field of drug discovery and development will understand that the precise source of test extracts or compounds is not critical to any of the screening procedures of the invention described here. Agents used in screens may include those known as therapeutics for the treatment of metabolic and/or gastrointestinal conditions. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N. H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have the desired activity further fractionation of the positive lead extract is necessary to isolate molecular constituents responsible for the observed effect. Thus, the goal of the extraction, fractionation, and purification process is the careful characterization and identification of a chemical entity within the crude extract that treats or prevents a neurological defect. Methods of fractionation and purification of such heterogenous extracts are known in the art. If desired, compounds shown to be useful as therapeutics are chemically modified according to methods known in the art.

### Differentiation and Culture System

Differentiation of human enteroendocrine cells, an essential epithelial cell type within the gastrointestinal system, may occur by driving intestinal stem cells to differentiate into the enteroendocrine lineage. One embodiment of the disclosure provides for a culture system for generating enteroendocrine cells through, for example, GATA4 activation/JNK inhibition and inhibition of FOXO1, factors previously not known to play a role during postnatal differentiation of these cells. Using organoid and/or enteroid biology and relative gene expression for endocrine cell markers, embodiments of the disclosure provide differentiation protocols using only agents such as small chemical molecules that target factors which affect postnatal differentiation by inducing gastrointestinal stem cells to undergo enteroendocrine lineage differentiation or development in vitro.

In some embodiments, the differentiation methods or systems disclosed here lead to the induction of large numbers of enteroendocrine cells, such as up to or equal to 10% of all cells (e.g., 1%, 2%, 3%, 4%, 5%, 6%, 7%, 8%, 9%), in contrast to less than 1% typically found in the native intestine. The large number of enteroendocrine cells may be found within the majority of all enteroids, such as up to or equal to 90% (e.g., 20%, 25%, 30%, 35%, 40%, 45%, 50%, 55%, 60%, 65%, 70%, 75%, 80%, 85%) or less than or equal to 100% (e.g., 95%, 90%, 85%, 80%, 75%, 70%, 65%, 60%, 55%, 50%).

Other embodiments of the differentiation and culture system provide for the maintenance of Wnt3a ligand in the culture system in order to induce enteroendocrine differentiation. In fact, the presence of Wnt3a was found to improve overall enteroendocrine differentiation. Accordingly, the differentiation and culture system of the disclosure includes Wnt3a for long-term maintenance of more than or equal to 1 day (e.g., 2 days, 3 days, 4 days, 5, days, 6, days, 7 days, 8 days, 9 days, 10 days, 11 days, 12 days, 13 days, 14 days, 15 days, 16 days, 17 days, 18 days, 19 days, 20 days, 21 days) of differentiating enteroids.

### Methods

In one embodiment, methods of human enteroendocrine (EE) cell differentiation comprising combining human gastrointestinal stem cells in a differentiation media of the disclosure and at least one modulating agent. For example, the modulating agent may be selected from a putative GATA4 activator or CB1 inverse agonist (e.g., Rim), a direct or indirect PDX1 activator (e.g., SP600125), a JNK inhibitor (e.g., SP600125), a FOXO1 inhibitor (e.g., AS1842856), or combinations thereof. The modulating agent may be in an amount effective to induce differentiation of the gastrointestinal stem cells, resulting in enteroendocrine cell differentiation. Some embodiments of the method of the disclosure may involve use of a modulating agent comprising a GATA4 activator and either a PDX1 activator or a JNK inhibitor to a differentiation media of the disclosure comprising the human gastrointestinal stem cells, resulting in increases in differentiated EE cells (and EE cell markers) and induction of multiple hormones, including but not limited to, serotonin, somatostatin, and GIP. Another embodiment of the disclosed method provides a modulating agent, such as a FOXO1 inhibitor (e.g., AS1842856), in an amount effective to result in increases in differentiated EE cells (and EE cell markers) and induction of multiple hormones, such as, for example, serotonin and somatostatin.

Another embodiment may be directed to methods of inducing hormones or different levels of hormonal expression by combining human gastrointestinal stem cells in differentiation media and a modulating agent selected from a putative GATA4 activator or CB1 inverse agonist (e.g., Rim), a PDX1 activator (e.g., SP600125), a JNK inhibitor (e.g., SP600125), a FOXO1 inhibitor (e.g., AS1842856), adenylate cyclase activator (e.g., forskolin (Fsk)), or combinations thereof, such as a putative GATA4 activator or CB1 inverse agonist (e.g., Rim), a PDX1 activator (e.g., SP600125) or a JNK inhibitor (e.g., SP600125), and a FOXO1 inhibitor (e.g., AS1842856). In some embodiments, the FOXO1 inhibitor may be added for an initial day(s) that are greater than or equal to 1 day (e.g., 2, 3, 4, 5, 6, 7) or ranging from 1-7 days (e.g., 1-6 days, 1-5 days, 1-4 days, 1-3 days, 1-2 days) of culture and a combination of only a GATA4 activator and either a PDX1 activator or a JNK inhibitor may be added for days greater than or equal to 1 day (e.g., 2, 3, 4, 5, 6, 7) or ranging from 1-7 additional days (e.g., 1-6, 1-5, 1-4 days, 1-3 days, 1-2 days) following the initial days, where after the initial days of culturing, no additional FOXO1 inhibitor is added. For example, a FOXO1 inhibitor may be added to human GISCs in a differentiation media of the disclosure for the initial 5 days and a combination of only a GATA4 activator and either a PDX1 activator or a JNK inhibitor may be added for another 5 days following the initial 5 days, where on Days 1-5 a FOXO1 inhibitor may be added and on Days 6-10 a combination of only a GATA4 activator and either a PDX1 activator or a JNK inhibitor may be added, without any FOXO1 inhibitor.

Yet a further embodiment may provide for a method of the disclosure, where only a FOXO1 inhibitor may be added for a predetermined initial set of days that is greater than or equal to 1 day (e.g., 2, 3, 4, 5, 6, 7) or ranging from 1-7 days (e.g., 1-6 days, 1-5 days, 1-4 days, 1-3 days, 1-2 days) of culture and a combination of a FOXO1 inhibitor, a GATA4 activator and either a PDX1 activator or a JNK inhibitor may be added for days greater than or equal to 1 day (e.g., 2, 3, 4, 5, 6, 7) or ranging from 1-7 additional days (e.g., 1-6, 1-5, 1-4 days, 1-3 days, 1-2 days) following the initial days, where after the initial days of culturing, FOXO1 inhibitor is continuously added. For example, a FOXO1 inhibitor may be added to human GISCs in a differentiation media of the disclosure for the initial 5 days and a combination of a FOXO1 inhibitor, a GATA4 activator, and either a PDX1 activator or a JNK inhibitor may be added for another 5 days following the initial 5 days, where on Days 1-5 a FOXO1 inhibitor may be added and on Days 6-10 a combination of a FOXO1 inhibitor, a GATA4 activator, and either a PDX1 activator or a JNK inhibitor may be added. In doing so, GIP expression is abolished, but serotonin secretion is enhanced.

Another embodiment provides for any of the methods of the disclosure that match or exceed EE cell numbers as compared to those found in normal GI tissue, induce secretion of different hormones, such as but not limited to, cholecystokinin (CCK), glucagon-like peptide-1 (GLP-1), glucose-dependent insulinotropic polypeptide (GIP), peptide YY (PYY), serotonin (5HT), and somatostatin (SST), and maintain healthy organoids for at least 10 days (e.g., 11 days, 12 days, 13 days, 14 days, 15 days, 16, days, 17 days, 18 days, 19 days, 20 days, 21 days; 11-21 days, 12-20 days, 13-19 days, 14-18 days, 15-17 days). In some embodiments, the modulating agent of the disclosure may be a small molecule or drug, which targets a transcription factor involved in GI development and/or in hormone production. Methods of the disclosure, in some embodiments, enhance the number of EE cells ex vivo and increase their viability.

As described in more detail in the Examples section (e.g., TABLES 1A and 1B), embodiments providing methods for inducing differentiation of human enteroids or organoids into enteroendocrine cells or enteroids or organoids comprising such cells, may comprise the steps of culturing the human enteroids or organoids in growth media (GM) (e.g., conditioned media containing Wnt3a, noggin, and R-spondin 3 (e.g., L-WRN conditioned media (50% v/v)); mammalian cell culture media with high concentrations of glucose, amino acids, and vitamins with additional nutrients (e.g., DMEM/F12 (45% v/v)); supplements (e.g., GlutaMax™ (1% v/v); N-2 Supplement (1% v/v); B-27 Supplement (1% v/v)); buffering agent (e.g., HEPES (10 mM)); antibiotic/antimicrobial (e.g., Primocin (100 μg/mL); Normocin (100 μg/mL)); small molecule modulating agent (e.g., TGF-βR inhibitor, A83-01 (500 nM); p38 MAPK inhibitor, SB202190 (10 μM)); antioxidant (e.g., N-Acetyl-cysteine (500 μM)); proliferating and/or differentiating agent (e.g., recombinant EGF (50 ng/mL); Nicotinamide (10 mM)); hormone (e.g., Human [Leu15] Gastrin I (10 nM)), where nicotinamide and SB202190 p38 MAPK inhibitor are specific only to the growth media, while the other components may also be found in the differentiation media of the disclosure. The differentiation media (DM) may comprise, for example, conditioned media containing Wnt3a, noggin, and R-spondin 3 (e.g., L-WRN conditioned media (50% v/v)); mammalian cell culture media with high concentrations of glucose, amino acids, and vitamins with additional nutrients (e.g., DMEM/F12 (45% v/v)); supplements (e.g., GlutaMax™ (1% v/v); N-2 Supplement (1% v/v); B-27 Supplement (1% v/v)); buffering agent (e.g., HEPES (10 mM)); antibiotic/antimicrobial (e.g., Primocin (100 μg/mL); Normocin (100 μg/mL)); small molecule modulating agent (e.g., TGF-βR inhibitor, A83-01 (500 nM); γ-secretase or Notch signaling inhibitor, DAPT (20 μM); HDAC6 inhibitor, Tubastatin-A (10 μM); MAP4K4 inhibitor, PF06260933 (6 μM); lysine-specific demethylase 1 inhibitor, Tranylcypromine (1.5 μM); a putative GATA4 activator or CB1 inverse agonist, Rimonabant (10 μM); JNK inhibitor or indirect PDX1 activator, SP600125 (10 μM); FOXO1 inhibitor, AS1842856 (100 nM)) antioxidant (e.g., N-Acetyl-cysteine (500 μM)); proliferating and/or differentiating agent (e.g., recombinant EGF (50 ng/mL); hormone (e.g., Human [Leu15] Gastrin I (10 nM); growth factor (e.g., Betacellulin (20 ng/mL)).

These methods of the disclosure may also be used in the treatment of a subject suffering from a metabolic and/or gastrointestinal disease or condition (e.g., irritable bowel syndrome, inflammatory bowel disease, Crohn's disease, ulcerative colitis, diabetes, including for example, Type II diabetes mellitus) or symptoms thereof, where the subject may suffer from an enteroendocrine cell dysfunction. Hormones in the GI system play a crucial role in the regulation of food intake, energy expenditure, glucose homeostasis, lipid metabolism, and a wide range of metabolic functions in response to the ingestion of food. These hormones may be altered in metabolic diseases, such as but not limited to, obesity and type 2 diabetes, and are thus proposed to be possible targets for the prevention or treatment of these diseases. Preparing functional enteroendocrine cells that express and secrete these hormones, such as but not limited to, cholecystokinin (CCK), glucagon-like peptide-1 (GLP-1), glucose-dependent insulinotropic polypeptide (GIP), peptide YY (PYY), serotonin (5HT), and somatostatin (SST), are important for treating subjects suffering from these diseases. In one embodiment, the GISCs of the method may be obtained or taken from the subject and used as the foundation to grow the enteroids/organoids or enteroendocrine cells ex vivo or in vitro, and then returned or administered to or implanted in the subject.

The present invention, in other embodiments, also provides methods of treating a metabolic and/or gastrointestinal disease and/or disorders or symptoms thereof which comprise administering a therapeutically effective amount of a pharmaceutical composition comprising a therapeutic agent or compound of the described herein to a subject (e.g., a mammal such as a human) suffering from the metabolic and/or gastrointestinal disease, which may also present as a dysfunction in enteroendocrine cells. Thus, one embodiment is a method of treating a subject suffering from or susceptible to a metabolic and/or gastrointestinal disease or disorder, such as those having an endocrine cell dysfunction, or symptom thereof. Metabolic diseases and/or disorders include, but are not limited to, obesity, diabetes (e.g., type 2 diabetes), while gastrointestinal diseases and/or disorders may include but are not limited to irritable bowel syndrome (IBS), inflammatory bowel disease (IBD), Crohn's disease, and ulcerative colitis. The method includes the step of administering to the subject, a therapeutic amount of an amount of an agent, such as but not limited to, isolated or purified human gastrointestinal stem cell (GISC)-generated functional enteroendocrine cells, enteroids, rectoids, or organoids comprising functional enteroendocrine cells, herein sufficient to treat the disease or disorder or symptom thereof, under conditions such that the disease or disorder is treated in a subject.

The methods described herein include administering to the subject (including a subject identified as in need of such treatment, e.g., a subject suffering from a metabolic or gastrointestinal disease or disorder) an effective amount of an agent, or a composition comprising such an agent of GISC-generated functional enteroendocrine cells or enteroids or rectoids or organoids comprising functional enteroendocrine cells, described herein, to produce an effect of ameliorating or reducing the treated disease or symptoms thereof. Identifying a subject in need of such treatment can be in the judgment of a subject or a health care professional and can be subjective (e.g. opinion) or objective (e.g. measurable by a test or diagnostic method).

Other embodiments may provide for transplanting. Transplanting may be auto-transplanting, allotransplanting, xenotransplanting, or any other type of transplanting. For example, transplanting can be autotransplanting. Transplanting can also be allotransplanting. “Allotransplantation” and its grammatical equivalents (e.g., allogenic transplantation) as used herein may encompass any procedure that involves transplantation, implantation, or infusion of cells, tissues, or organs into a recipient subject, where the recipient subject and donor subject are the same species but different individuals. Transplantation of the cells, organs, and/or tissues described herein can be used for allotransplantation into humans. Allotransplantation includes but is not limited to vascularized allotransplant, partially vascularized allotransplant, unvascularized allotransplant, allodressings, allobandages, and allostructures.

“Autotransplantation” and its grammatical equivalents (e.g., autologous transplantation) as used herein can encompass any procedure that involves transplantation, implantation, or infusion of cells, tissues, or organs into a recipient subject, where the recipient subject and donor subject is the same individual. Transplantation of the cells, organs, and/or tissues described herein can be used for autotransplantation into humans Autotransplantation includes but is not limited to vascularized autotransplantation, partially vascularized autotransplantation, unvascularized autotransplantation, autodressings, autobandages, and autostructures. In one embodiment, gastrointestinal stem cells, isolated cells, isolated crypts or enteroids from the duodenum of a subject may be grown and differentiated in vitro or ex vivo, and when a sufficient number and/or effective amount of functional enteroendocrine cells are obtained by methods described herein, they may be returned, administered, or transplanted into the same subject by any methods, including those presently known in the art.

After transplanting, the transplanted cells may be functional in the recipient subject, or when autotransplanted, in the same subject. In some instances, the determination of functionality may dictate whether transplantation was successful. For example, the transplanted cells may be functional for at least 1, 2, 3, 4, 5, 6, 7, 8, 9, 10 or more days after transplantation indicating the success of the transplantation. This can also indicate that there is no rejection of the transplanted cells, tissues, and/or organs.

Immunosuppressive therapy can comprise any treatment that suppresses the immune system in order to alleviate, minimize, or eliminate transplant rejection in a recipient, particularly in xenotransplantation and allotransplantation. Non-limiting examples of immunosuppressive drugs of immunosuppressive therapy that may be administered to a recipient subject before, during and/or after transplant include, MMF (mycophenolate mofetil (Cellcept)), ATG (anti-thymocyte globulin), anti-CD154 (CD40L), anti-CD40 (2C10, ASKP1240, CCFZ533X2201), alemtuzumab (Campath), anti-CD20 (rituximab), anti-IL-6R antibody (tocilizumab, Actemra), anti-IL-6 antibody (sarilumab, olokizumab), CTLA4-Ig (Abatacept/Orencia), belatacept (LEA29Y), sirolimus (Rapimune), everolimus, tacrolimus (Prograf), daclizumab (Ze-napax), basiliximab (Simulect), infliximab (Remicade), cyclosporin, deoxyspergualin, soluble complement receptor 1, cobra venom factor, compstatin, anti C5 antibody (eculizumab/Soliris), methylprednisolone, FTY720, everolimus, leflunomide, anti-IL-2R-Ab, rapamycin, anti-CXCR3 antibody, anti-ICOS antibody, anti-OX40 antibody, and anti-CD122 antibody. Combinations of one or more than one immunosuppressive agents/drugs may also be used together, simultaneously, or sequentially. These immunosuppressive agents/drugs may also be useful for induction therapy or maintenance therapy. In some embodiments, the same or different drugs may be used during induction and maintenance stages. In some cases, daclizumab (Zenapax) can be used for induction therapy and tacrolimus (Prograf) and sirolimus (Rapimune) can be used for maintenance therapy. Daclizumab (Zenapax) can also be used for induction therapy and low dose tacrolimus (Prograf) and low dose sirolimus (Rapimune) can be used for maintenance therapy. Immunosuppression can also be achieved using non-drug regimens including, but not limited to, whole body irradiation, thymic irradiation, and full and/or partial splenectomy. These techniques can also be used in combination with one or more immuno-suppressive drugs. A medical professional

The therapeutic methods of the invention (which include prophylactic treatment) in general comprise administration of a therapeutically effective amount of the agents described herein, such as an agent or compound of a particular formulae disclosed herein, to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may also be used in the treatment of any other diseases in which enteroendocrine cell dysfunction may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a metabolic and/or gastrointestinal disease or a disease associated with an enteroendocrine dysfunction, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Kits

In one embodiment, the invention provides for kits comprising GISC-derived enteroendocrine cells, or enteroids or organoids comprising such cells, as described in the invention. In another embodiment, the invention provides reagents (e.g., growth media, differentiation media, cell culture media, supplements, modulating agents (e.g., inhibitors or activators), antibiotics/antimicrobials, growth factors, nutrients) for obtaining GISC-derived enteroendocrine cells, or enteroids or organoids described herein, alone or in combination with directions for the use of such reagents. Associated with such kits may be a notice in the form prescribed by a governmental agency regulating the manufacture, use or sale of biological products, which notice reflects approval by the agency of manufacture, use or sale for human administration. Instructions for preparing the GISC-derived enteroendocrine cells, or enteroids, rectoids, or organoids may also accompany such kits.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

Since there are challenges in converting human intestinal stem cells (ISCs) into functional EE cells, ex vivo, there has been limited progress in elucidating the role of EE cells in disease pathogenesis and in harnessing their therapeutic potential. To address this, small molecule targeting of key transcriptional regulators, GATA4, JNK and FOXO1, known to mediate endodermal development and hormone production, together with directed differentiation of human ISCs from the duodenum and rectum were employed. A marked induction of EE cell differentiation and gut-derived expression and secretion of hormones, for example, cholecystokinin (CCK), glucagon-like peptide-1 (GLP-1), glucose-dependent insulinotropic polypeptide (GIP), peptide YY (PYY), serotonin (5HT), and somatostatin (SST), upon treatment with various combinations of three small molecules: rimonabant, SP600125 and AS1842856 was observed. Robust differentiation strategies capable of driving human EE cell differentiation was a critical step towards understanding these essential cells and the development of cell-based therapeutics.

### Example 1: Differentiation Media Induces CHGA Expression in Human Enteroids

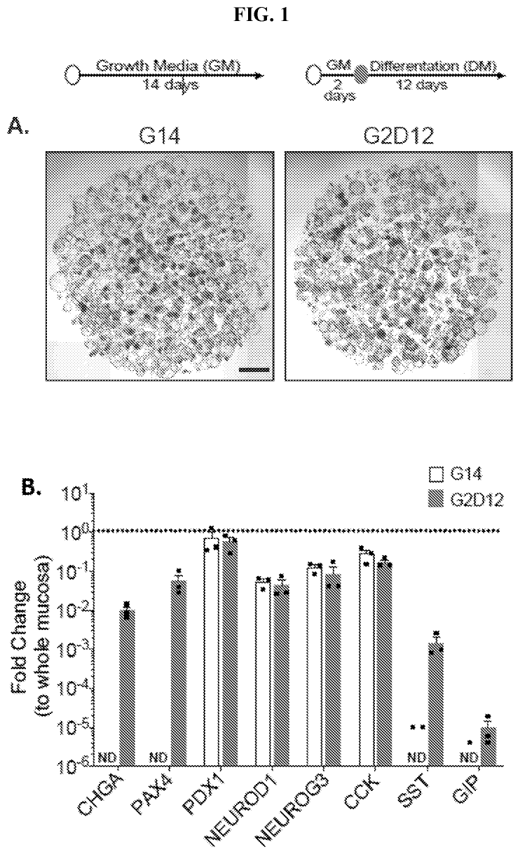

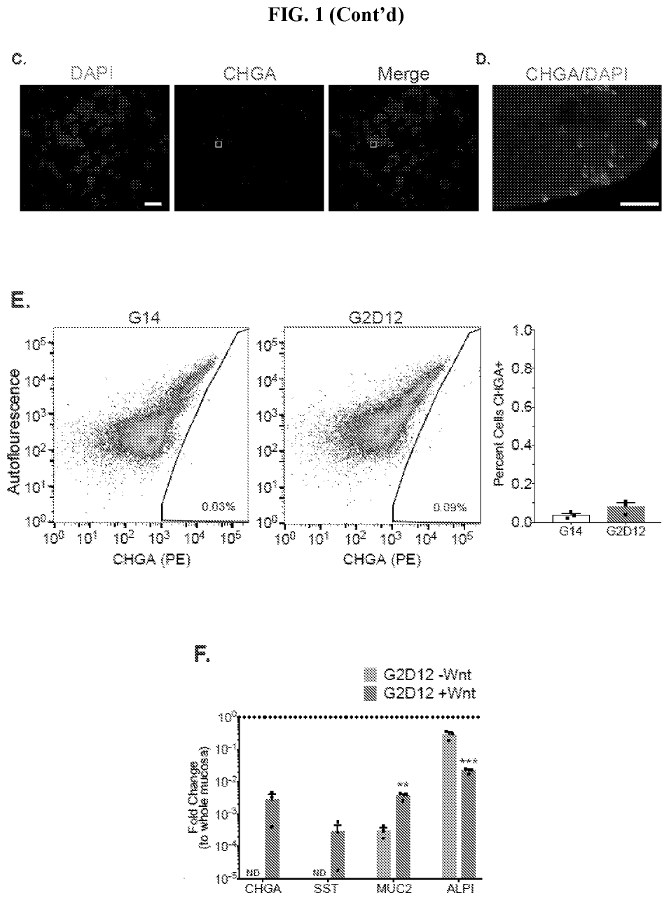

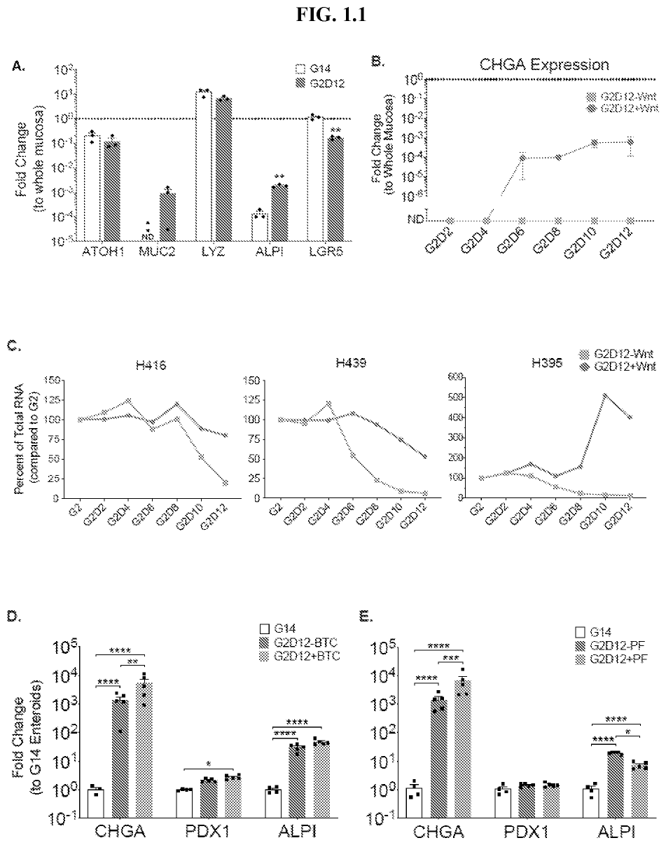

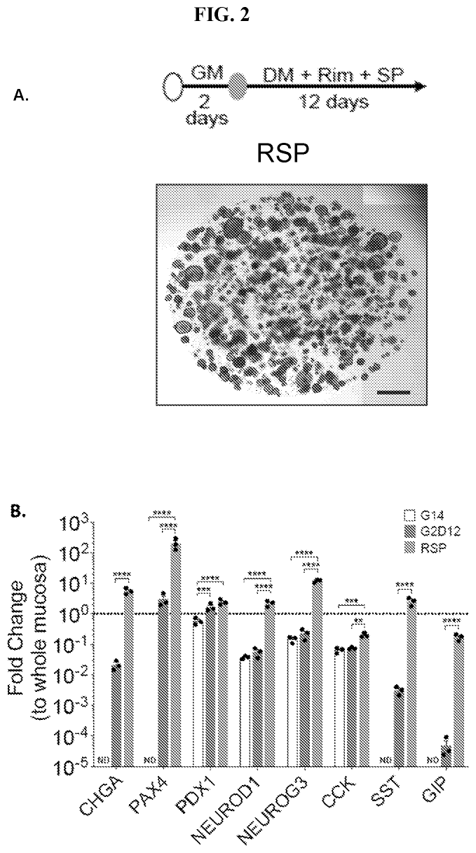

To optimize human enteroendocrine (EE) cell differentiation, a differentiation media (DM, TABLES 1A and 1B) was developed by combining specific components from published protocols used to induce EE cells (Sato T, Stange D E, Ferrante M, et al. Gastroenterology 2011; 141:1762-72; Yin X, Farin H F, van Es J H, et al. Nat Methods 2014; 11:106-12; Beumer J, Artegiani B, Post Y, et al. Nat Cell Biol 2018; 20:909-916; VanDussen K L, Marinshaw J M, Shaikh N, et al. Gut 2015; 64:911-20), including Wnt3a and two additional small molecules associated with endocrine cell differentiation: (1) betacellulin, a ligand of both EGFR and the ErbB4 receptor (Pagliuca F W, Millman J R, Gurtler M, et al. Cell 2014; 159:428-39; Roy, S. A. et al. World J Gastroenterol 18, 1579-1589, doi:10.3748/wjg.v18.i14.1579 (2012); Harskamp, L. R., Gansevoort, R. T., van Goor, H. & Meijer, E. The epidermal growth factor receptor pathway in chronic kidney diseases. Nat Rev Nephrol 12, 496-506, doi:10.1038/nrneph.2016.91 (2016))^{41,42} and (2) PF06260933 (PF), a small molecule inhibitor of MAP4K4 (Zhao X, Mohan R, Ozcan S, et al. J Biol Chem 2012; 287:31155-64)43. Human enteroids were first cultured in growth media (GM, TABLE 1A) for two days, allowing for intestinal stem cell (ISC) expansion, followed by DM for 12 days (G2D12). Enteroids maintained for 14 days in GM (G14) were used as controls to assess for changes in gene expression. In contrast to previous strategies aimed at generating EE cells over a five-day period (Sato T, Stange D E, Ferrante M, et al. Gastroenterology 2011; 141:1762-72; Beumer J, Artegiani B, Post Y, et al. Nat Cell Biol 2018; 20:909-916), exposure to DM for 12 days was compatible with maintenance of enteroid structural integrity (FIG. 1A), possibly due to the continued presence of WNT3a.

TABLE 1A and TABLE 1B provide growth factors, supplements, and small molecules common to both growth media (GM) and differentiation media (DM) that are initially listed. Individual components in the left column correspond to growth media; those in the right column correspond to differentiation media for each of TABLE 1A and TABLE 1B, and if there is no distinction, then the component spans both the GM and DM columns. The L-WRN cell line was used to produce conditioned media containing Wnt3a, noggin, and R-spondin 3. The HA-R-Spondin1-Fc 293T cell line was used to produce conditioned media with only R-spondin 1. This conditioned media, with supplemented noggin (100 ng/mL) was used to make a Wnt3a-free differentiation media.

TABLE 1A
Growth and Differentiation Media Components
ENTEROID

| | |
|---|---|
| Growth Media | Differentiation Media |

L-WRN conditioned media (50% v/v)
DMEM/F12 (45% v/v)
Glutamax (1% v/v)
N-2 Supplement (1% v/v)
B-27 Supplement (1% v/v)
HEPES (10 mM)
Primocin (100 μg/mL)
Normocin (100 μg/mL)
A83-01 (500 nM)
N-Acetyl-cysteine (500 μM)
Recombinant Murine EGF (50 ng/mL)
Human [Leu15] Gastrin I (10 nM)

| | |
|---|---|
| Nicotinamide (10 mM) | DAPT (20 μM) |
| SB202190 (10 μM) | Betacellulin (20 ng/mL) |
| | Tubastatin-A (10 μM) |
| | PF06260933 (6 μM) |
| | Tranylcypromine (1.5 μM) |
| | Rimonabant (10 μM) |
| | SP600125 (10 μM) |
| | AS1842856 (100 nM) |

TABLE 1B
Growth and Differentiation Media Components
RECTOID

| | |
|---|---|
| Growth Media | Differentiation Media |

L-WRN conditioned media (65% v/v)
DMEM/F12 (30% v/v)
Glutamax (1% v/v)
N-2 Supplement (1% v/v)
B-27 Supplement (1% v/v)
HEPES (10 mM)
Primocin (100 μg/mL)
Normocin (100 μg/mL)
A83-01 (500 nM)
N-Acetyl-cysteine (500 μM)
Recombinant Murine EGF (50 ng/mL)
Human [Leu15] Gastrin I (10 nM)

| | |
|---|---|
| Nicotinamide (10 mM) | DAPT (20 μM) |
| SB202190 (10 μM) | Betacellulin (20 ng/mL) |
| Prostaglandin-E2 (10 nM) | Tubastatin-A (10 μM) |
| | PF06260933 (6 μM) |
| | Tranylcypromine (1.5 μM) |

To quantify the effectiveness of DM to induce EE cell differentiation, gene expression of CHGA and other lineage markers, including but not limited to, mucin 2 (MUC2, goblet cells), lysozyme (LYZ, Paneth cells), intestinal alkaline phosphatase (ALPI, enterocytes), and leucine-rich repeat-containing G-protein coupled receptor 5 (LGR5, ISCs) were profiled. In addition, the expression of transcription factors required for duodenal EE differentiation and function (PDX1, PAX4, NEUROG3, and NEUROD1), as well as hormones secreted from the duodenum (e.g., SST and GIP) were assessed. To allow for relative comparison to native tissue expression levels, total RNA from whole mucosal biopsies was included in each qPCR analysis. Exposure of enteroids to G2D12 induced consistent expression of CHGA, PAX4, SST, and GIP, with significantly higher levels of ALPI and significantly lower expression of LGR5 when compared to G14 (FIG. 1B and FIG. 1.1A). Despite G2D12's ability to induce EE and enterocyte markers when compared to G14, their overall expression levels remained considerably lower than whole mucosa. Levels of PDX1, NEUROD1, NEUROG3, CCK, GIP, ATOH1, and LYZ were unchanged compared to G14, with MUC2 showing a trend towards higher expression in G2D12 enteroids (FIG. 1B and FIG. 1.1A). Finally, despite induction in CHGA mRNA levels in response to G2D12, analysis of CHGA protein using immunofluorescent staining and flow cytometric analysis revealed fewer than 0.1% of all cells to be CHGA+, showing a trend towards higher expression compared with G14 (FIG. 1B-1E). Non-limiting enteroendocrine markers (including, for example, lineage markers) useful in various embodiments of the disclosure include: atonal BHLH transcription factor 1 (ATOH1); intestinal alkaline phosphatase (ALPI); cholecystokinin (CCK); chromogranin A (CHGA); glucose-dependent insulinotropic peptide (GIP); leucine-rich repeat-containing G-protein coupled receptor 5 (LGR5); lysozyme (LYZ); mucin 2 (MUC2); neuronal differentiation 1 (NEUROD1); neurogenin 3 (NEUROG3); paired box 4 (PAX4); pancreatic and duodenal homeobox 1 (PDX1); somatostatin (SST); and combinations thereof. Removal of WNT3a had been shown to aid EE differentiation (Sato T, Stange D E, Ferrante M, et al. Gastroenterology 2011; 141:1762-72; Basak O, Beumer J, Wiebrands K, et al. Cell Stem Cell 2017; 20:177-190 e4; Beumer J, Artegiani B, Post Y, et al. Nat Cell Biol 2018; 20:909-916). However, WNT3a removal was found to be detrimental to both EE differentiation and long-term viability, as noted by a lack of CHGA expression at any time during the differentiation protocol and a marked decrease in total RNA levels with time. The data indicated that removal of WNT3a was not necessary for human EE cell differentiation. Analysis of enteroids cultured in DM without WNT3a (G2D12-Wnt) revealed undetectable expression levels of CHGA and SST, reduced MUC2 expression, and increased ALPI expression when compared with enteroids differentiated with WNT3a (G2D12+Wnt) (FIG. 1F). To determine whether CHGA expression was increased at an earlier time-point during the 14 day differentiation protocol, time-course studies were performed and CHGA expression was found to be undetectable in enteroids cultured in G2D12-Wnt, while those exposed to G2D12+Wnt showed expression after six days of starting DM (FIG. 1.1B). Furthermore, two of the three enteroid lines exposed to G2D12-Wnt showed low total RNA levels around the eighth day of differentiation, consistent with failure to maintain these lines (FIG. 1.1C). Therefore, the presence of WNT3a in DM was determined to be necessary for sustaining enteroids in long-term culture (14 days) and was not detrimental to EE differentiation. The impact of the inclusion of betacellulin and PF in the disclosed DM was evaluated and both factors led to increased expression of EE cell markers compared with GM and DM without betacellulin or PF (FIG. 1.1D and FIG. 1.1E). Together, these data indicate that the disclosed differentiation protocol markedly induced expression of some EE cell marker genes (e.g., CHGA and PAX4), but was not sufficient to induce a significant increase in the number of CHGA+EE cells compared to undifferentiated controls.

Enteroids or organoids derived from normal human duodenum that were grown ex vivo or in vitro in differentiation media (DM) to induce EE cell differentiation, along with multiple combinations of different small molecules targeting GATA4, JNK and FOXO1. EE differentiation was evaluated by performing qPCR analysis on multiple EE markers including, but not limited to, chromogranin A (CHGA), neuronal differentiation 1 (NEUROD1), neurogenin 3 (NEUROG3), glucose-dependent insulinotropic polypeptide (GIP), and somatostatin (SST), using whole human duodenal mucosa as a reference. EE cell differentiation was further evaluated by immunofluorescent staining of CHGA, which included identifying the proportion of total enteroids with CHGA-positive cells as well as flow cytometric analysis to identify the total percentage of CHGA-positive cells. Hormone production was also evaluated through immunofluorescent staining of enteroids and hormone concentrations within conditioned media. EE differentiation of human enteroids using currently available or previously described methods induced low mRNA levels of CHGA, GIP, and SST as compared to whole duodenal mucosa, and did not improve with the removal of Wnt3a. Further, flow cytometric analysis demonstrated a marginal, non-significant increase in the number of CHGA-positive cells when compared to undifferentiated enteroids. The addition of a GATA activator (e.g., rimonabant, Rim), a putative GATA activator (e.g., Rim), or CB1 inverse agonist (e.g., Rim) and a PDX1 activator (e.g., SP600125, SP) or a JNK inhibitor (e.g., SP600215) induced mRNA expression of CHGA, NEUROD1, NEUROG3, SST, and GIP to levels at or surpassing native duodenal mucosa, yielding approximately 1.0% of the total cell population expressing CHGA, a 5-10 fold increase compared to the base differentiation media. Separately, the addition of a FOXO1 inhibitor (e.g., AS1842856, AS) induced similar mRNA expression trends as Rim and SP (RSP), yielding approximately 5.0% CHGA-positive cells on flow cytometric analysis. Analysis of GIP, SST, and serotonin (5HT) immunofluorescence showed that these hormones were produced in both RSP and AS conditions, with hormone secretion evaluation from conditioned media suggesting that AS promoted serotonin (5HT) production and secretion, while RSP promoted GIP secretion. Notably, AS treatment followed by RSP yielded the largest amount of GIP secretion.

Therefore, GATA4, JNK, and FOXO1 were found to play critical roles in human EE differentiation and hormone production, improving on current methods of differentiation without the use of direct genetic alteration.

### Example 2: Treatment with Rimonabant and SP600125 Induces EE Lineage Differentiation

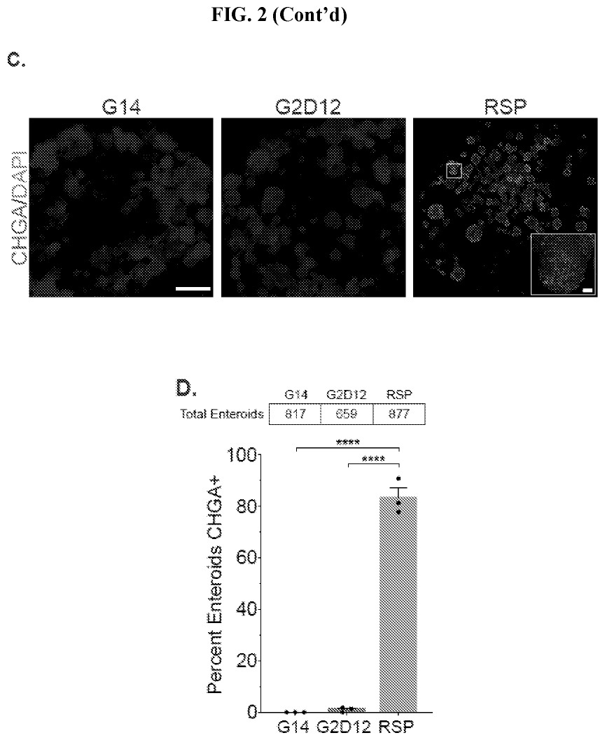

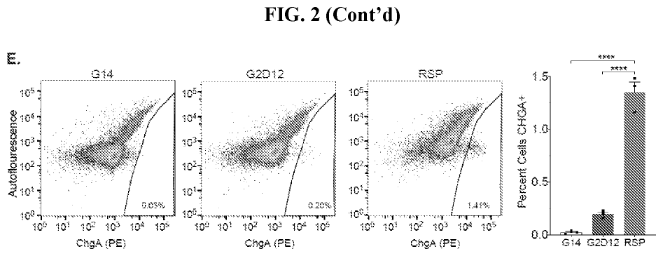

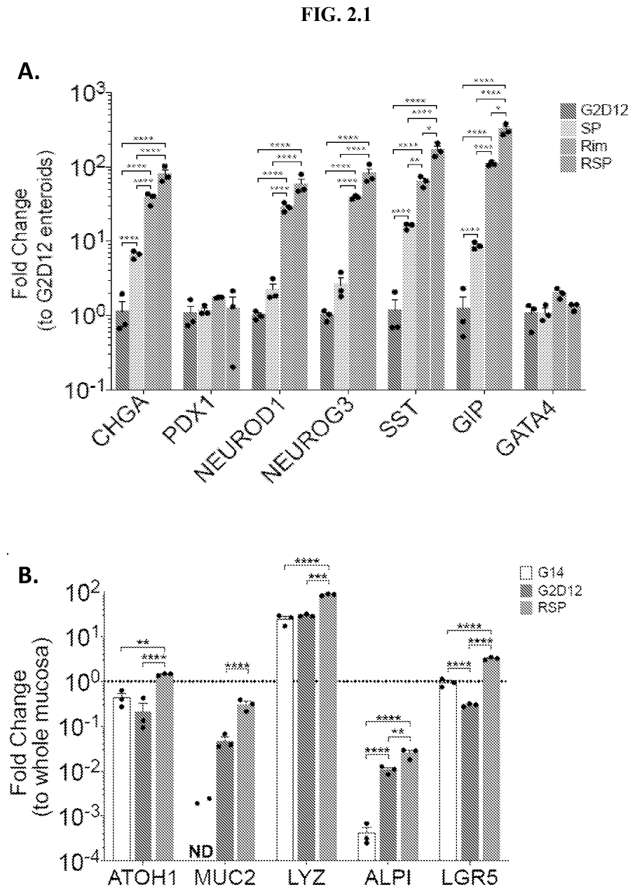

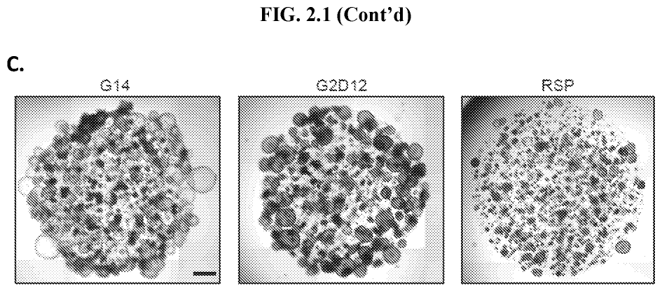

Small molecules that might further induce EE cell differentiation, such as, transcriptional regulators involved in gastrointestinal development and hormone regulation, were determined. To identify additional strategies that might further induce EE cell differentiation, small molecules that were shown to target GATA4 and PDX1 activity, key transcriptional regulators involved in gastrointestinal development and hormone regulation, were tested. To induce GATA4 activation, first, rimonabant (Rim), a highly selective cannabinoid receptor type I (CB1) antagonist that has been shown to increase human serum GIP levels (Sathyapalan, T. et al. Clin Endocrinol (Oxf) 72, 423-425, doi:10.1111/j.1365-2265.2009.03643.x (2010))⁴⁴ and that is structurally identical to Compound 7, a molecule which was previously shown to increase the activity of GATA4-NKX2-5 transcriptional activity in vitro (Valimaki, M. J. et al. J Med Chem 60, 7781-7798, doi:10.1021/acs.jmedchem.7b00816 (2017).45, was utilized. In parallel, the small molecule SP600125 (SP) was used to inhibit JNK signaling, which has been shown to suppress PDX1 activity (Tang, C. et al. Endocrinology 159, 3643-3654, doi:10.1210/en.2018-00566 (2018); Kaneto, H. et al. Curr Diabetes Rev 1, 65-72 (2005))^{31,32} Separately, both Rim and SP induced expression of multiple EE lineage markers (CHGA, NEUROD1, NEUROG3, SST, and GIP) when added to DM, with Rim having a much larger effect (FIG. 2.1A). Together, the combination of Rim and SP (RSP) yielded even further increases in SST and GIP expression compared to Rim or SP alone. Both PDX1 and GATA4 were unchanged under all experimental conditions (FIG. 2.1A1A). Based on these data, Rim and SP were used in combination for all subsequent experiments. The addition of RSP to DM maintained overall enteroid structural integrity during the 14-day differentiation protocol (FIG. 2A; FIG. 2.1B). Moreover, compared to enteroids grown in G14 and G2D12, treatment with RSP led to the upregulation of multiple EE markers (e.g., CHGA, PAX4, NEUROD1, NEUROG3, CCK, SST, and GIP) to levels approximating whole mucosa (FIG. 2B). Other lineage markers were also increased with RSP exposure, including, e.g., ATOH1, MUC2, ALPI, and LGR5 when compared to G14 and G2D12 (FIG. 2.1B). Immunofluorescence staining for CHGA showed multiple positive cells within individual enteroids (FIG. 2C), with a large majority of enteroids (83%) containing CHGA+ cells (FIG. 2D). By comparison, only 1% of enteroids grown in G2D12 were CHGA+(FIG. 2D). Quantitative flow cytometric analysis revealed 1.3% of all cells treated with RSP were CHGA+, almost seven times the number seen with G2D12 alone (FIG. 2E).

Activation of GATA4, inhibition of JNK, and inhibition of FOXO1 were found to all play important roles in the directed differentiation of human EE cells from duodenal ISCs. Prior to this disclosure, the differentiation of EE cells using the enteroid culturing system had mainly been studied in the context of Wnt, Notch, MAPK and BMP signaling, with inhibition of these pathways driving gastrointestinal stem cells into the secretory lineage, and subsequent removal of p38 MAPK inhibition leading to EE cell differentiation (Basak, O. et al. Cell Stem Cell 20, 177-190 e174, doi:10.1016/j.stem.2016.11.001 (2017); Chen, C., Fang, R., Davis, C., Maravelias, C. & Sibley, E. Am J Physiol Gastrointest Liver Physiol 297, G1126-1137, doi: 10.1152/ajpgi.90586.2008 (2009))⁹⁻¹⁶. Despite these important advances, the overall efficiency of EE differentiation in the majority of these other studies is hard to estimate due to their strong reliance on gene expression analysis with little data at the protein level. However, in embodiments of the disclosure described here, robust induction of EE differentiation was shown here and assessed using gene and protein expression as well as hormone secretion (e.g., cholecystokinin (CCK), glucagon-like peptide-1 (GLP-1), glucose-dependent insulinotropic polypeptide (GIP), peptide YY (PYY), serotonin (5HT), and somatostatin (SST)), following treatment with small molecules targeting, for example, FOXO1, GATA4, and JNK, at levels that approach the native duodenal mucosa.

### Example 3: Treatment with AS1842856 Induces Duodenal EE Lineage Differentiation

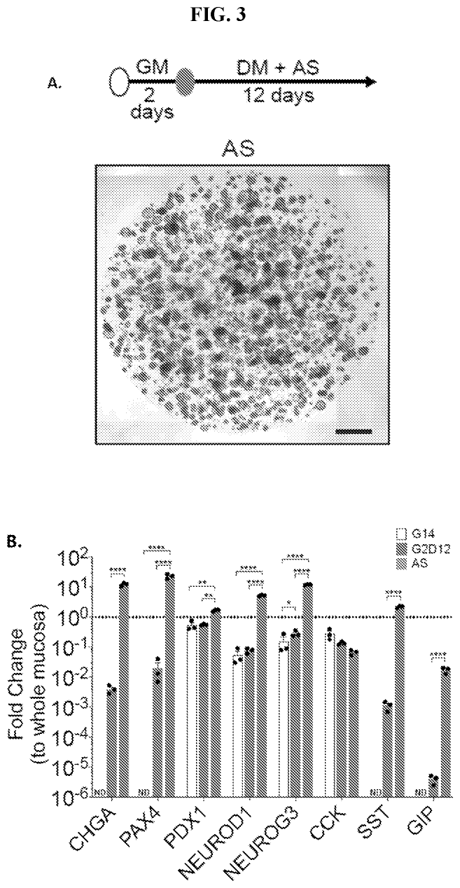

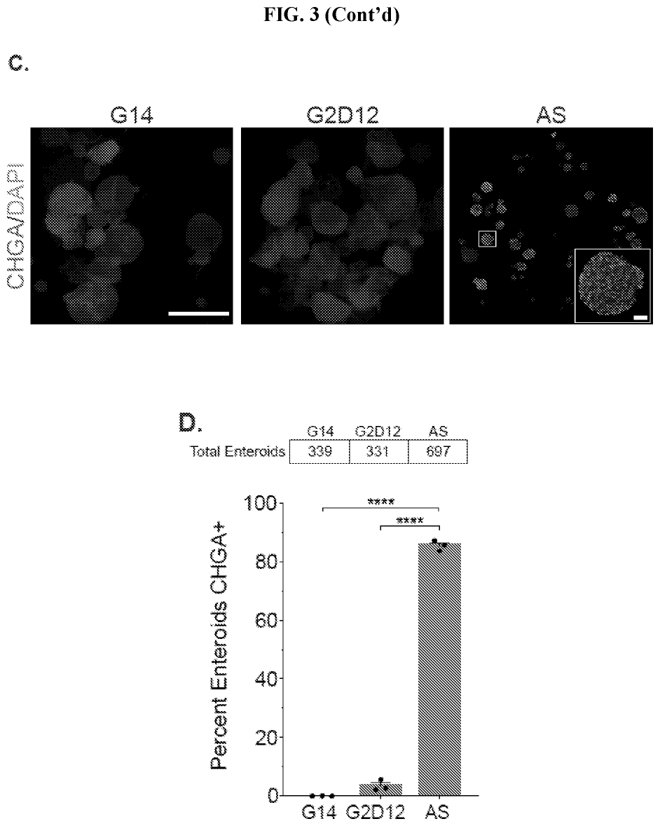

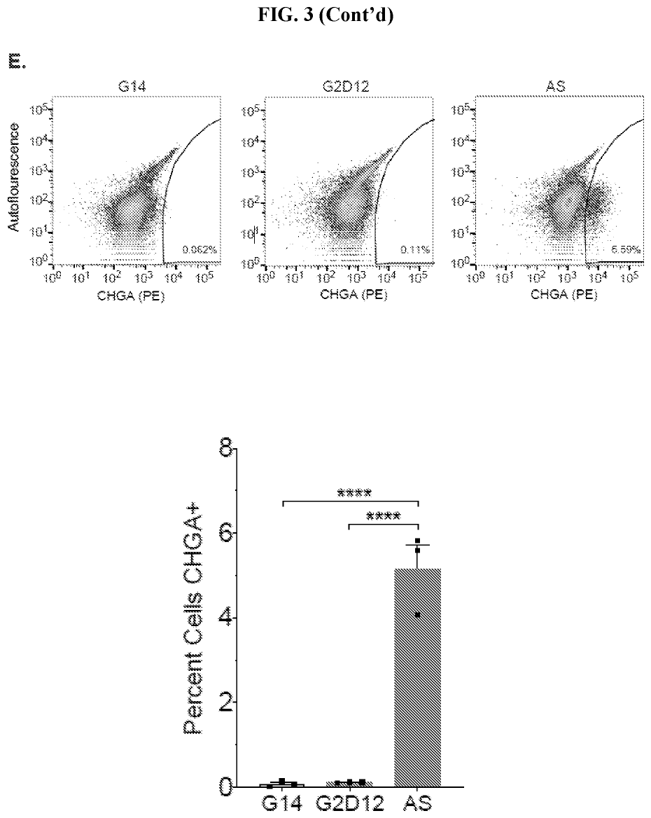

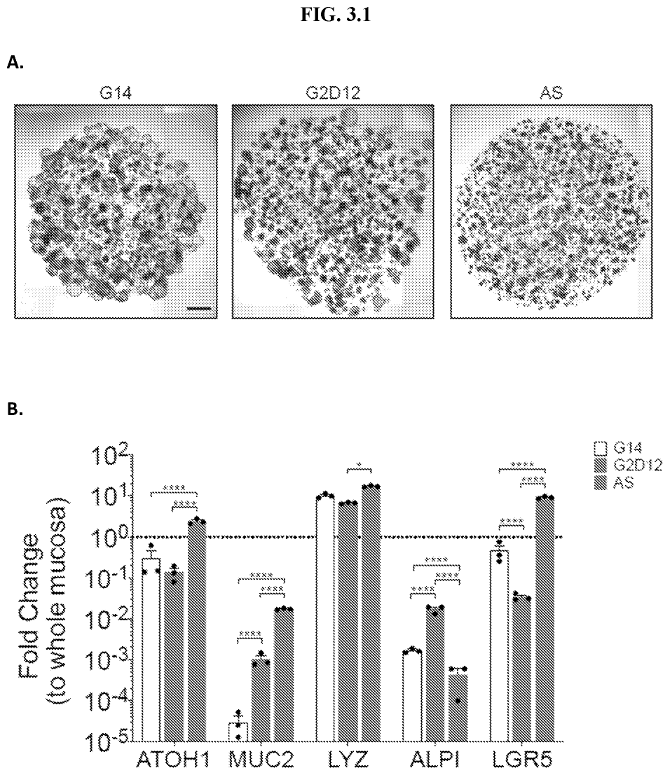

To assess whether inhibition of FOXO1 could facilitate the differentiation of EE cells in enteroids, AS1842856 (AS), a FOXO1 inhibitor (Yu F, Wei R, Yang J, et al. Exp Cell Res 2018; 362:227-234)⁴⁶, was added to the disclosed differentiation protocol. Addition of AS to the DM culture conditions led to the formation of small, spherical enteroids (FIG. 3A; FIG. 3.1A). Compared to enteroids grown in G14 and G2D12, AS treatment led to the upregulation of multiple EE markers (CHGA, PAX4, PDX1, NEUROD1, NEUROG3, SST and GIP), many to levels approximating whole mucosa (FIG. 3B). In addition, AS increased expression of other secretory and ISC markers, including ATOH1, MUC2, LYZ, and LGR5, when compared to G14 and G2D12 (FIG. 3.1B). Interestingly, the use of AS reduced expression of the enterocyte marker ALP1, when compared to G14 and G2D12, suggesting a possible role for FOXO1 inhibition in the induction of the secretory lineage (FIG. 3.1B). Immunofluorescent staining revealed CHGA+ cells within a large majority of individual enteroids (85%) (FIGS. 3C and 3D). By comparison, only 3% of enteroids grown in G2D12 had CHGA+ cells (FIG. 3D). Quantitative flow cytometric analysis revealed 5.2% of all cells exposed to AS to be CHGA+, almost 50 times the number seen with G2D12 alone (FIG. 3E).

### Example 4: Treatment with Rimonabant/SP600125 or AS1842856 Promote Distinct Enteroid Differentiation Trajectories

To better assess the transcriptomic distinctions between cells from the various culture conditions, single cell RNA sequencing (scRNA-seq) on the 10× platform combined with sample hashing was utilized, allowing the assessment of the reproducibility of the findings from distinct donors. Enteroids from three individuals were grown in G2D12, RSP or AS, for a total of nine samples. After the 14-day protocol, enteroids were isolated and processed into a single-cell suspension and their condition and patient source were marked using Hashtag antibodies prior to pooling the samples together (Stoeckius, M. et al. Genome Biol 19, 224, doi:10.1186/s13059-018-1603-1 (2018).)⁴⁷. After labeling, cells were processed, and single-cell transcriptomes were generated.

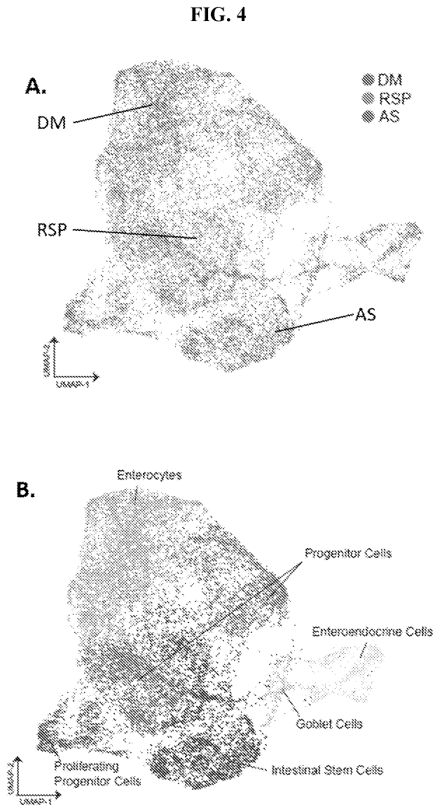

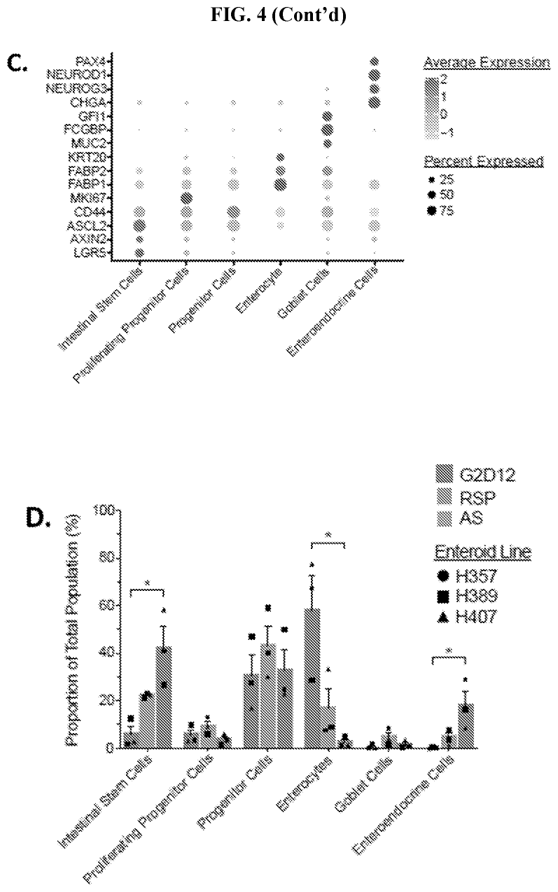

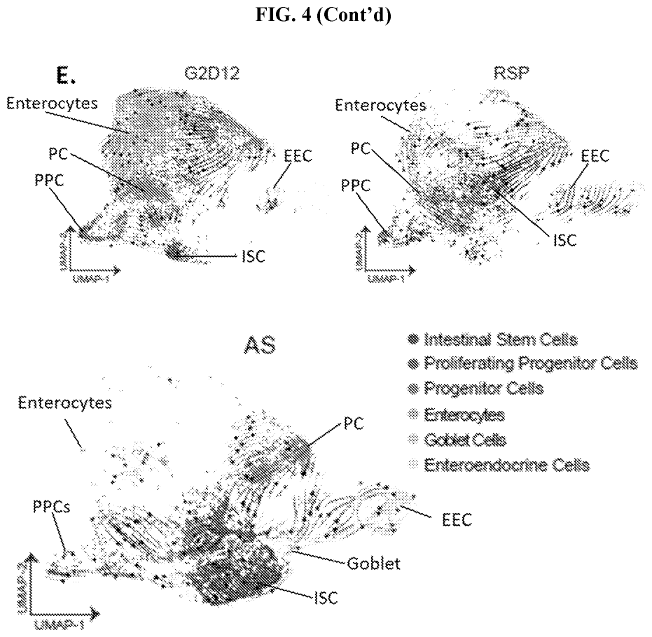

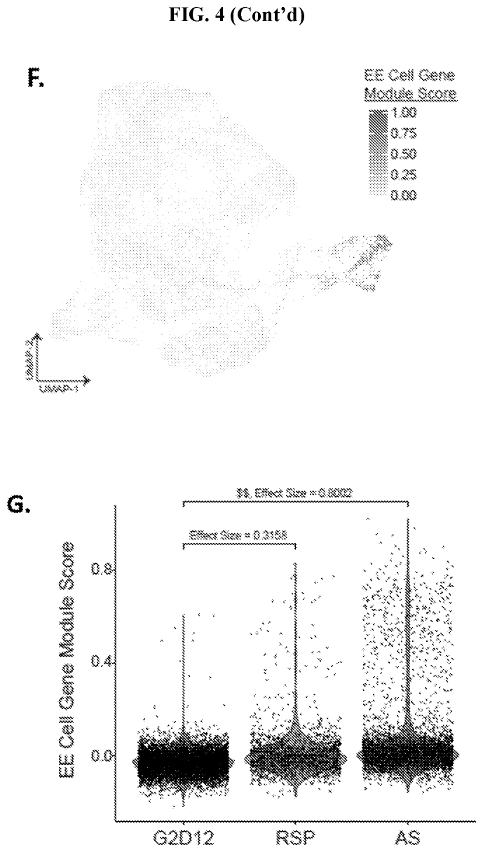

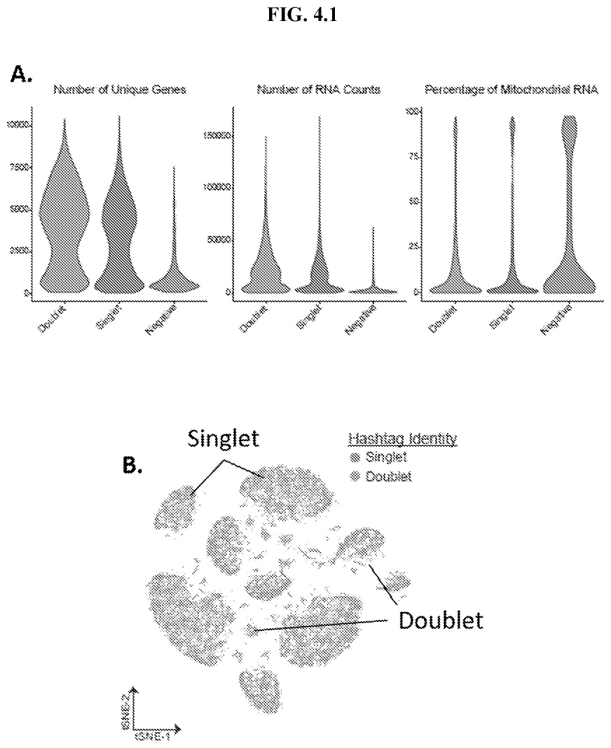

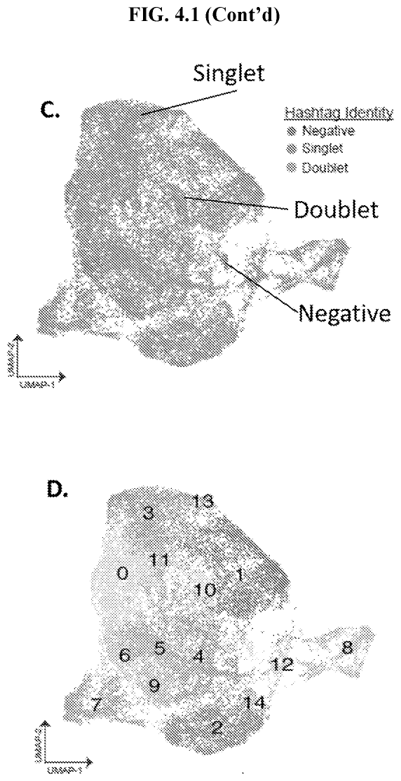

By using hashtag labeling, the cells were categorized into singlets (significant detection of one antibody), doublets (significant detection of more than one antibody) or negative droplets (no significant detection of any antibodies). Negative droplets were found to have a reduced number of both detected genes and Unique Molecular Identifiers (UMIs), supporting the idea that the majority of these datapoints do not correspond to an individual cell (FIG. 4.1A). In contrast, both singlets and doublets had appreciable numbers of both genes and UMIs detected. In addition, they had similar UMIs, percentage of mitochondrial RNA, and detected gene distributions. However, upon dimensional reduction, singlets and doublets formed distinct groupings in both tSNE visualization (FIG. 4.1B), as well as UMAP visualization and clustering (FIG. 4.1C and FIG. 4.1D), indicating that although they had similar RNA content, singlets likely corresponded to true singlets; whereas doublets corresponded to multiple cell types analyzed together in a single droplet.

After removal of doublets, negative droplets, and low-quality cells (gene count<200, percentage of mitochondrial RNA>25%), the resulting dataset consisted of 14,767 cells spread across the three different culture conditions. UMAP visualization revealed distinct cell separation between G2D12 and AS treatment with minimal overlap between the two populations. In contrast, enteroid treatment with RSP generated populations that overlapped with the two other culture conditions (FIG. 4A). To further characterize the developing populations within each treatment, Louvain clustering was performed and identified 15 clusters within the combined dataset (FIG. 4.1D). Of these 15 clusters, some were composed of cells sourced from all three culture conditions, such as clusters 2, 4, 9, and 7; whereas other clusters were primarily formed by cells from one treatment group, such as cluster 8 (AS) and cluster 3 (G2D12). To assist with manual annotation of each cluster, the top markers of each Louvain cluster (Wilcoxon rank sum test, p_{adj}<1.38×10⁻²¹ for displayed markers)⁴⁸ were calculated. A heatmap was generated showing the top 10 most differentially expressed genes per Louvain cluster compared to all other clusters. Specifically, differentially expressed genes were calculated using the Wilcoxon rank sum test and the top 10 genes of each cluster were selected based on their log fold enrichment in the specified cluster compared to all other clusters. Heatmap values corresponded to row-scaled Pearson residuals of gene expression that was normalized using regularized negative binomial regression (data not shown). By using these markers, as well as classical markers of various epithelial populations (Haber, A. L. et al. Nature 551, 333-339, doi:10.1038/nature24489 (2017); Mead, B. E. et al. BMC Biol 16, 62, doi:10.1186/s12915-018-0527-2 (2018)⁴⁹′5°, the data was broadly classified into five populations of epithelial cells, including stem cells, progenitor cells, enterocytes, goblet cells and enteroendocrine cells (FIG. 4B). ISCs were identified by expression of LGR5, AXIN2 and ASCL2 (Barker, N. Nat Rev Mol Cell Biol 15, 19-33, doi:10.1038/nrm3721 (2014))⁵′. Enterocytes were identified by the expression of KRT20, FABP2, and FABP1, goblet cells, by the expression of MUC2, FCGBP, and GF11, and EE cells by expression of CHGA, NEUROG3, NEUROD1, and PAX4 (van der Flier, L. G. & Clevers, H. Annu Rev Physiol 71, 241-260, doi:10.1146/annurev.physiol.010908.163145 (2009); Noah, T. K., Donahue, B. & Shroyer, N. F. Exp Cell Res 317, 2702-2710, doi: 10.1016/j.yexcr.2011.09.006 (2011))^{52,53}. Finally, progenitor cells were identified as having a mixture of stem cell markers, albeit at a lower level than the ISCs, while also expressing mature enterocyte markers. Proliferating cycling progenitors were distinguished from other progenitor cells by the expression of MKI67 (FIG. 4C); however, some progenitor markers were also lowly expressed in secretory cell populations, suggesting that these groups likely also contain secretory progenitors.

To understand how different culture conditions impacted enteroid development and differentiation, the proportions of the various cell types across each condition (FIG. 4D) were compared. Treatment with G2D12 primarily gave rise to cells of the absorptive lineage, with 58% identified as enterocytes. The remaining cells exposed to G2D12 consisted of stem cells (5.7%), proliferating progenitor cells (5.4%), and progenitor cells (30%). G2D12 did not give rise to secretory cells, with almost no detection of goblet cells (0.6%) or EE cells (0.2%). In contrast to G2D12, treatment with either RSP or AS led to a decrease in the proportion of enterocytes (RSP: 7%, AS: 2.5%) and an increase in stem cells (RSP: 22%, AS: 42%), goblet cells (RSP: 4.6%, AS: 1.5%), and EE cells (RSP: 4.3%, AS: 18%), with significance differences noted between G2D12 and AS conditions for stem cells, enterocytes and EE cells.

To further characterize the differentiation path of each culture condition, scVelo was used to model the RNA velocity and differentiation trajectory of each cell type (Bergen, V., Lange, M., Peidli, S., Wolf, F. A. & Theis, F. J. Nat Biotechnol 38, 1408-1414, doi:10.1038/s41587-020-0591-3 (2020))⁵⁴. Briefly, RNA velocity analysis pipelines, such as scVelo, leverage the fact that scRNA-seq can distinguish between un-spliced and spliced variants of each gene. Since recently transcribed RNA was present as an un-spliced variant which was then spliced into its mature form before eventually being degraded, it was possible to calculate the dynamic kinetics of each individual gene's expression across all cells within the dataset based on the ratio of un-spliced to spliced RNA (Bergen, V., Lange, M., Peidli, S., Wolf, F. A. & Theis, F. J. Nat Biotechnol 38, 1408-1414, doi:10.1038/s41587-020-0591-3 (2020); La Manno, G. et al. Nature 560, 494-498, doi:10.1038/s41586-018-0414-6 (2018))^{54,55}. scVelo used the collection of gene kinetics to infer the direction and speed of cell state changes on a population level. This method was applied to each culture condition and generated vector estimates of cellular transition overlaid on each UMAP (FIG. 4E). In all three conditions, the majority of vector arrows initiated from a stem cell cluster in the lower half of the UMAP. In the G2D12 culture condition, cells primarily moved towards proliferating progenitors and progenitor cells, before moving towards the enterocyte cluster. In contrast, in AS treated cells, the vector arrows proceeded through a completely different trajectory, with the majority of vectors moving towards the goblet cell cluster and then into the EE cluster. Cells treated with RSP revealed vector arrows in patterns that resembled both G2D12 and AS treated cells, suggesting it may promote early differentiation of both secretory and absorptive cell types. Finally, a small proportion of vector arrows suggested a “backwards” trajectory, with movement from a differentiated enterocyte into a progenitor population. This behavior may have been indicative of de-differentiation or possibly movement towards cell death as the cells lose features associated with mature cell types (Asfaha, S. et al. Cell Stem Cell 16, 627-638, doi:10.1016/j.stem.2015.04.013 (2015); Tetteh, P. W. et al. Cell Stem Cell 18, 203-213, doi:10.1016/j.stem.2016.01.001 (2016))^{56,57}. All three culture conditions had distinct differentiation potentials, with AS and RSP giving rise to an increased proportion of EE cells compared to G2D12.

To verify that the EE cells identified in the enteroid system of the disclosure resembled those found in situ, dataset was compared with a reference gene set from isolated mouse EE cells (Haber, A. L. et al. Nature 551, 333-339, doi:10.1038/nature24489 (2017)⁴⁹. This list encompassed 77 genes, including conventional markers such as CHGA, NEUROG3, and the enzyme TPHI (data not shown). Briefly, a heatmap of marker genes significantly enriched in in vivo enteroendocrine (EE) cells isolated from the mouse small intestinal epithelial cell atlas was generated. Heatmap values were calculated using similar methods seen in FIG. 4E and may not represent significantly enriched genes. An EE Cell Gene Module Score was calculated for each cell in the scRNA dataset based on the number of shared features with reference EE gene set, which revealed exclusive marking of cells within the EE cell cluster (FIG. 4F). In addition, when looking across culture conditions, RSP and AS showed an increased effect size in relation to G2D12, with AS having a larger effect size than RSP (FIG. 4G). These results underscore differences observed between the three culture conditions described in previous results and further demonstrated that EE cells generated using these protocols resembled those found within the native tissue.

### Example 5: Treatment with AS1842856 Followed by Rimonabant and SP600215 Increases GIP Expression in Human Enteroids

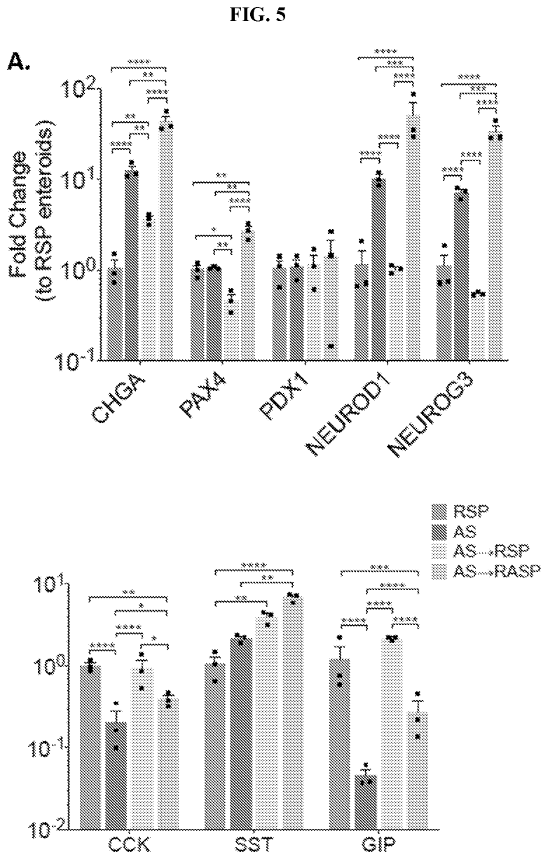

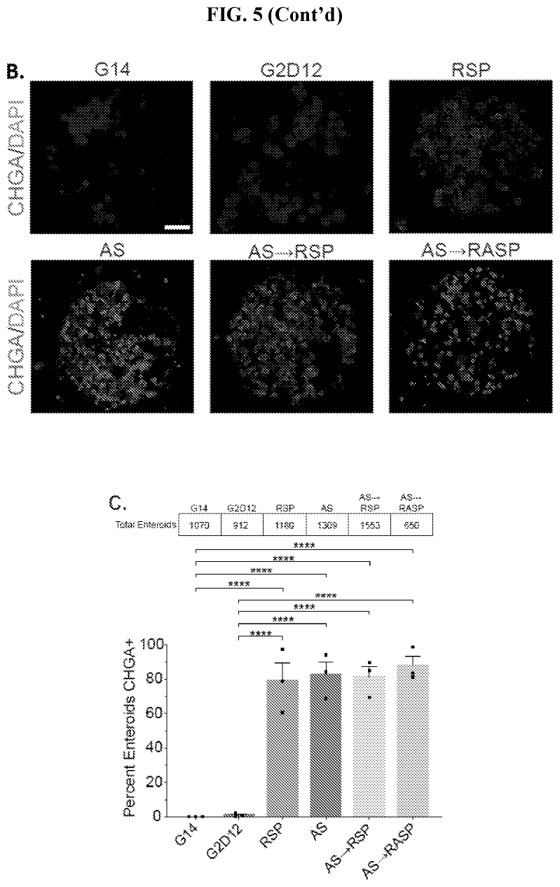

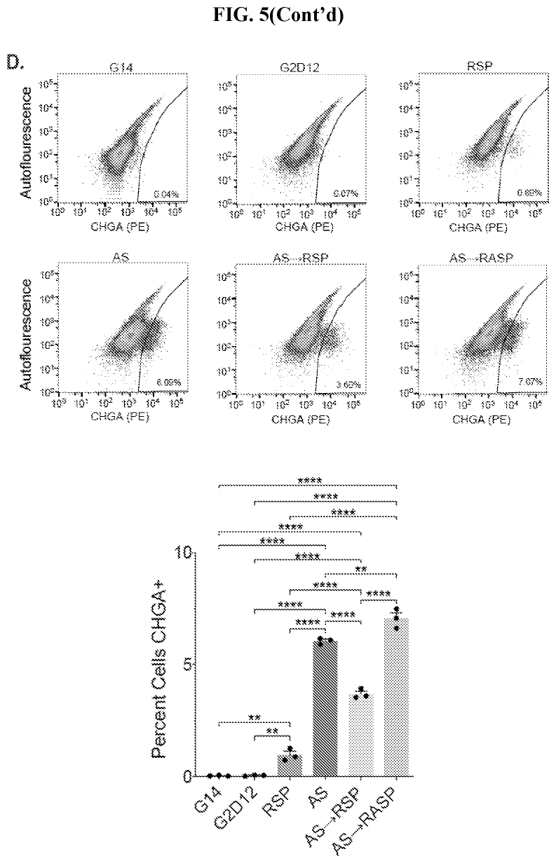

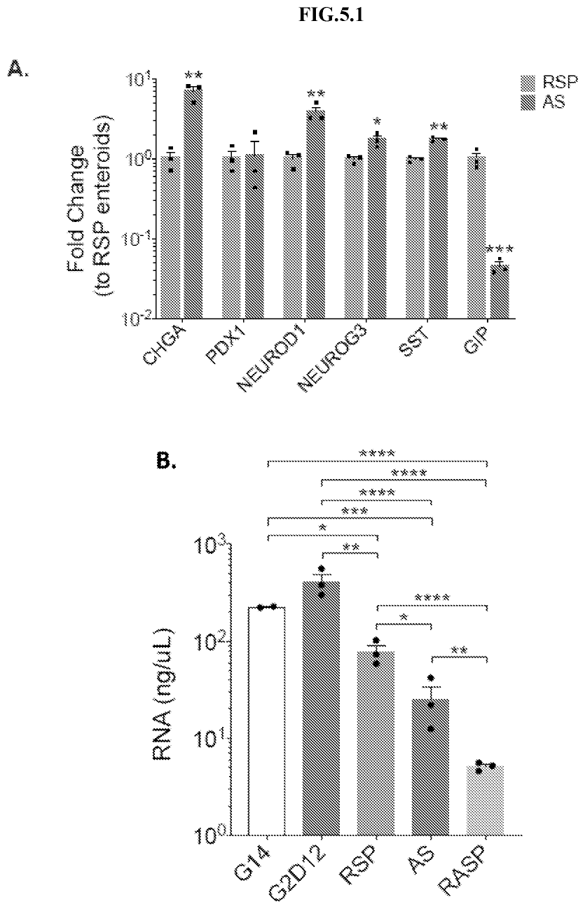

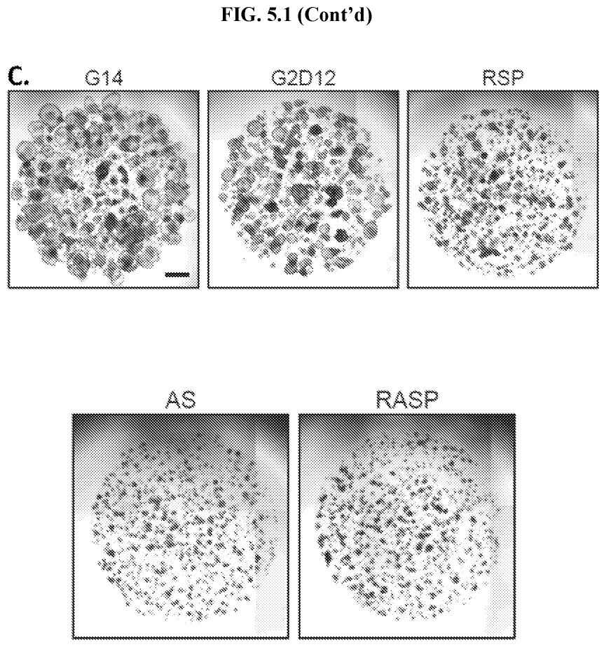

Compared to RSP-treated enteroids, AS treatment led to more robust EE differentiation, based on overall induction of multiple EE cell markers and the larger fraction of CHGA+ cells (FIGS. 2-4). Direct comparison of the two protocols revealed that AS exposure led to higher expression of CHGA, NEUROD1, NEUROG3, and SST (FIG. 5.1A), consistent with the higher percentage of CHGA+ cells (FIGS. 2 and 3). In contrast, RSP induced higher expression of GIP compared to AS (FIG. 5.1A), suggesting that GATA4 activation/JNK inhibition is a more potent inducer of GIP than FOXO1 inhibition. Given these results, the combination of Rim, AS, and SP (RASP) was expected to further increase expression of both CHGA and GIP. Exposure of enteroids to RASP for the full duration of the differentiation protocol, however, was not compatible with viable enteroids, as evidenced by their irregular morphology and extremely low RNA content (FIG. 5.1B and FIG. 5.1C).

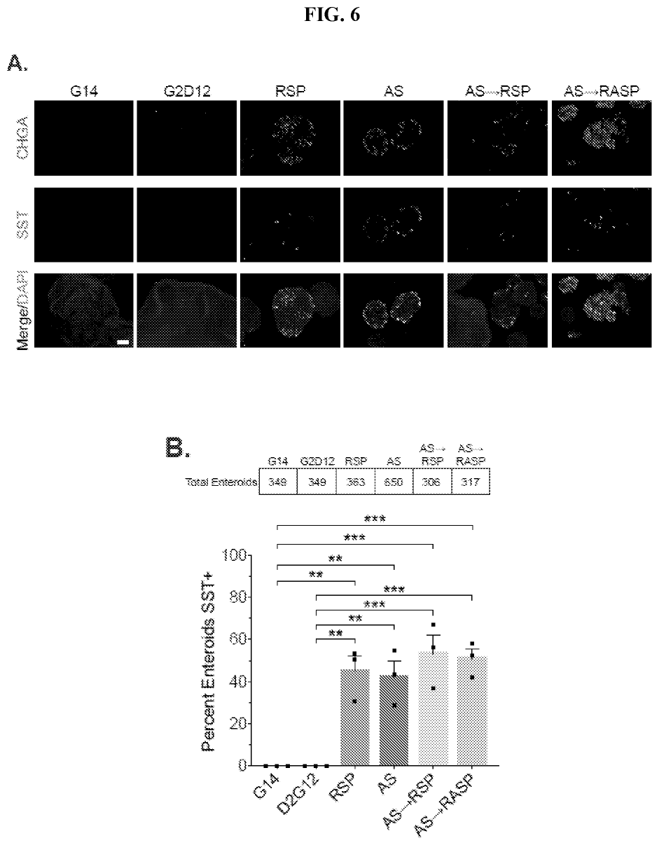

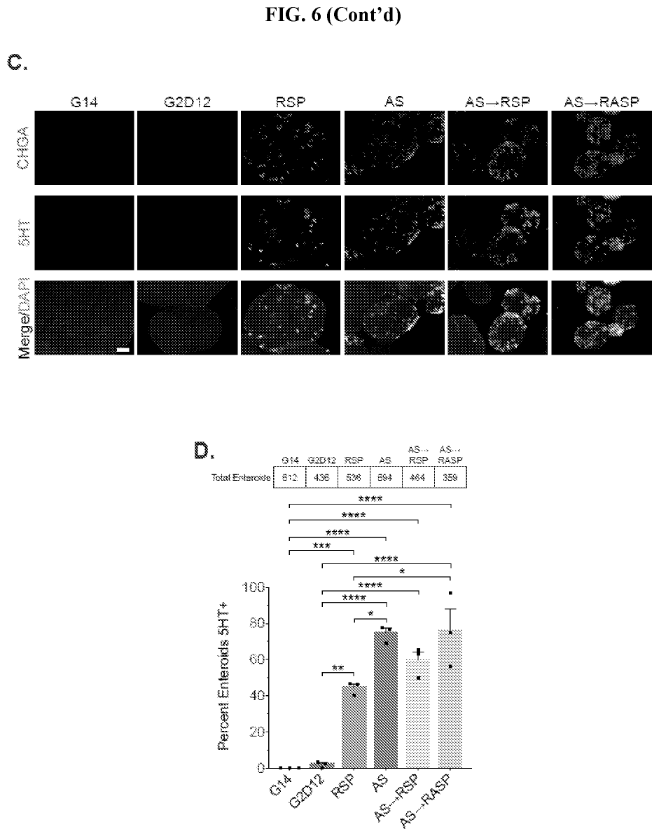

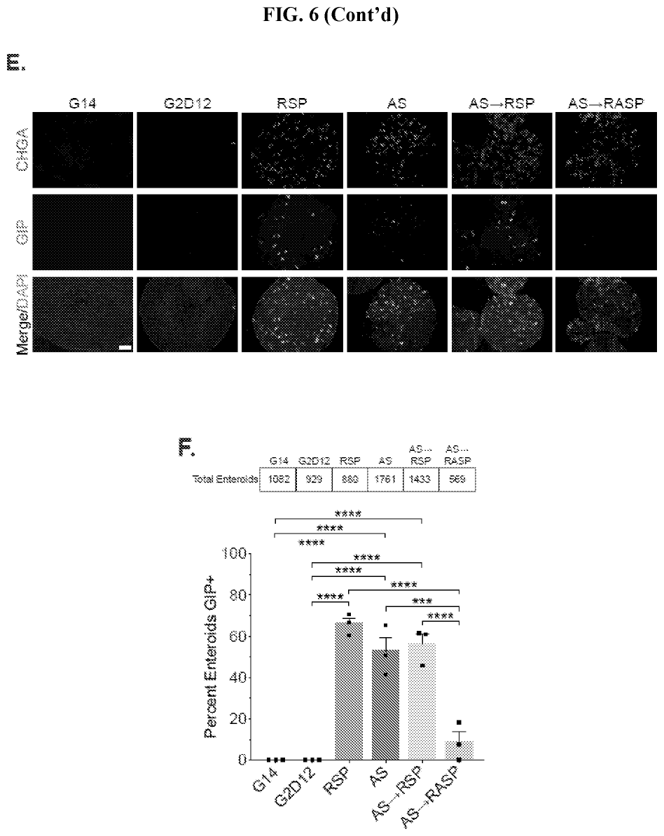

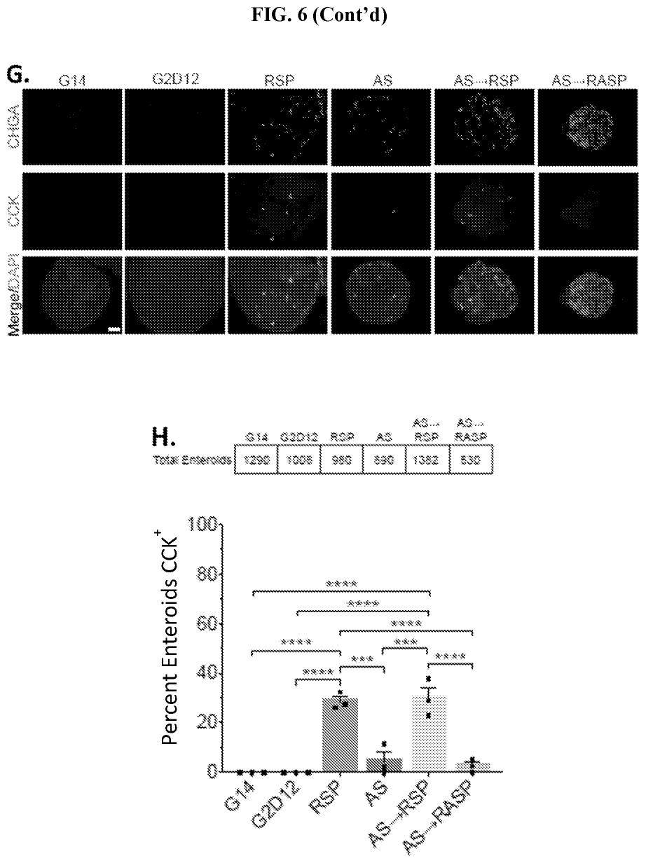

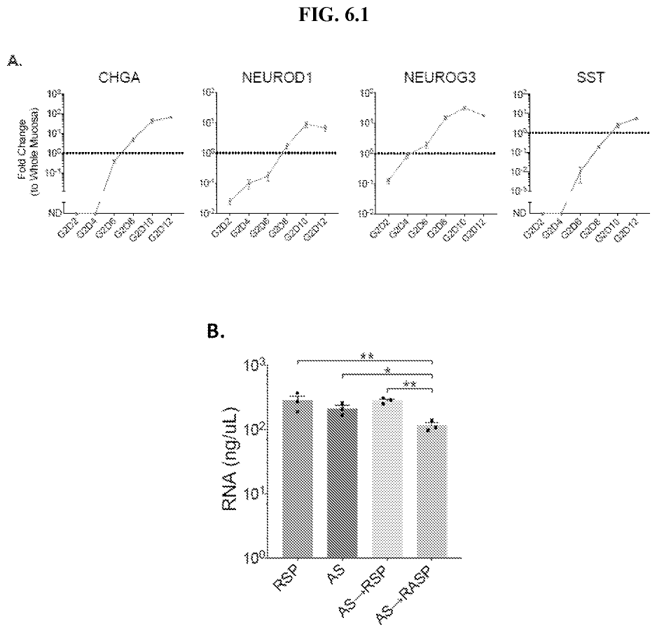

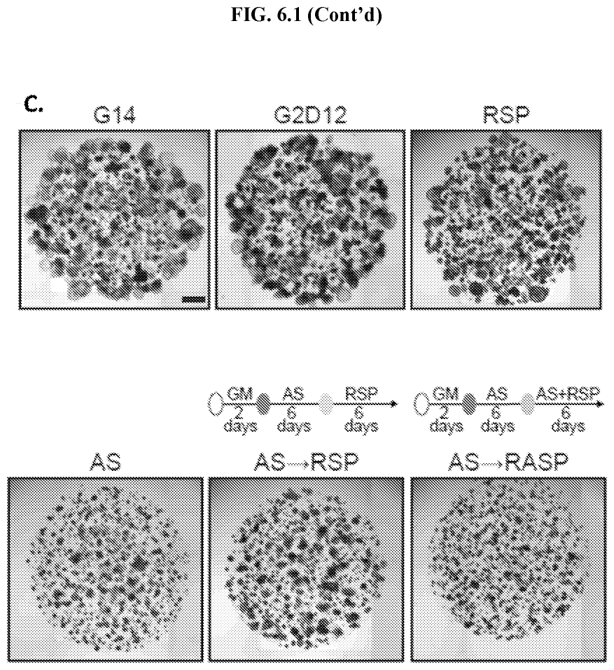

The impact of adding RSP after exposure to AS was tested, because it was thought that AS treatment would shunt a larger proportion of cells into the EE lineage, with the later addition of RSP, which induced GIP expression in more cells than RSP alone. In order to identify the appropriate time for the addition of RSP, a time-course analysis of AS-treated enteroids was performed and found that multiple transcription factors were required for EE differentiation, including NEUROD1 and NEUROG3, which showed increased expression around the fourth day of differentiation. Following this, both CHGA and SST had detectable transcript levels by the sixth day of differentiation (FIG. 6.1A). Given these data, AS treatment for six days followed by subsequent exposure to RSP was hypothesized to lead to increased GIP expression compared to RSP alone. To test this, two differentiation strategies were utilized: (1) switching from AS to RSP at day six of differentiation (AS→RSP) or (2) adding RSP to AS at day six of differentiation (AS→RASP). Morphologically, AS→RASP produced smaller enteroids compared to other conditions (FIG. 6.1B); however, RNA concentrations were consistently above the minimum threshold of 10 ng/μL, suggesting improved viability over exposure to RASP for the full duration of the differentiation protocol (FIG. 6.1C). Enteroids treated with AS→RASP showed 2-3-fold higher gene expression levels of most EE cell markers when compared to AS (FIG. 5A). In contrast, enteroids treated with AS→RSP showed gene expression changes that were either similar or higher than RSP alone, but were significantly lower when compared to AS alone, aside from PAX4 and PDX1 (FIG. 5A). Interestingly, although AS→RSP induced expression of CCK and GIP similar to RSP alone, AS→RASP induced expression levels significantly less than RSP alone (FIG. 5A).

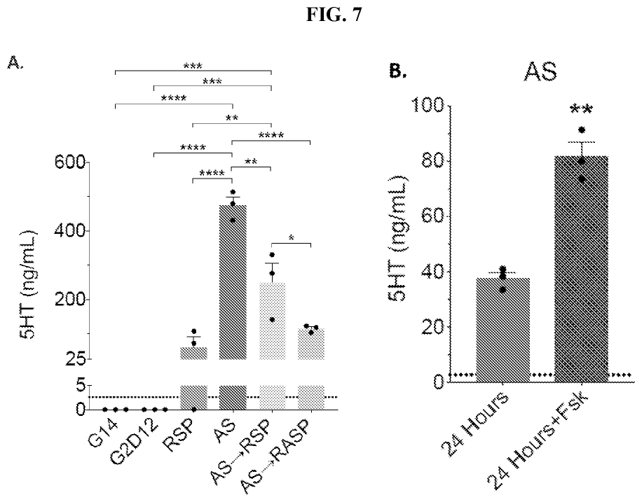

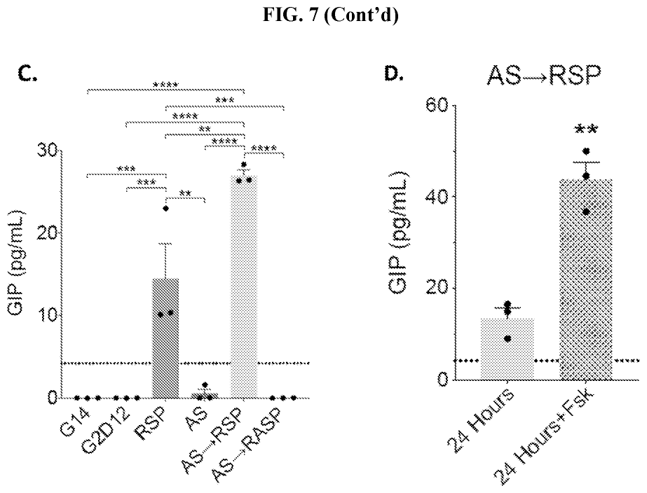

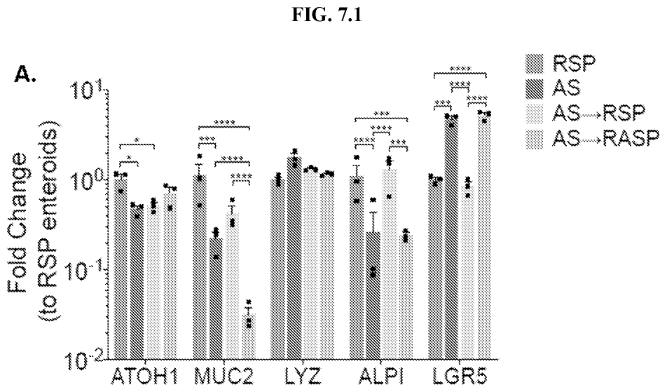

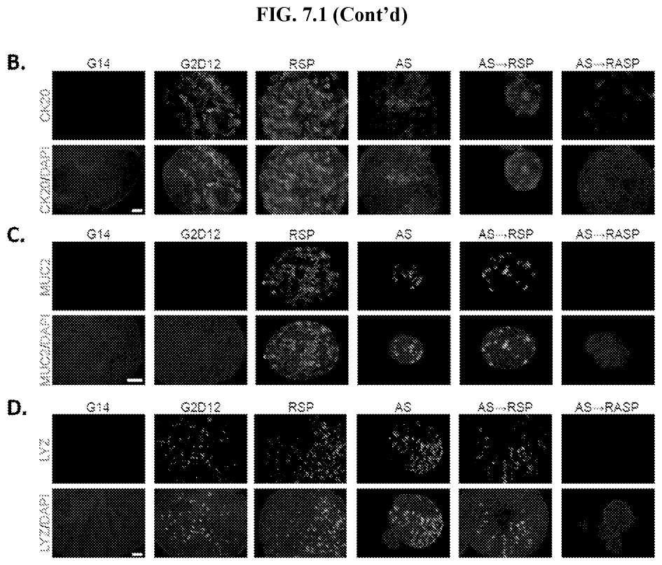

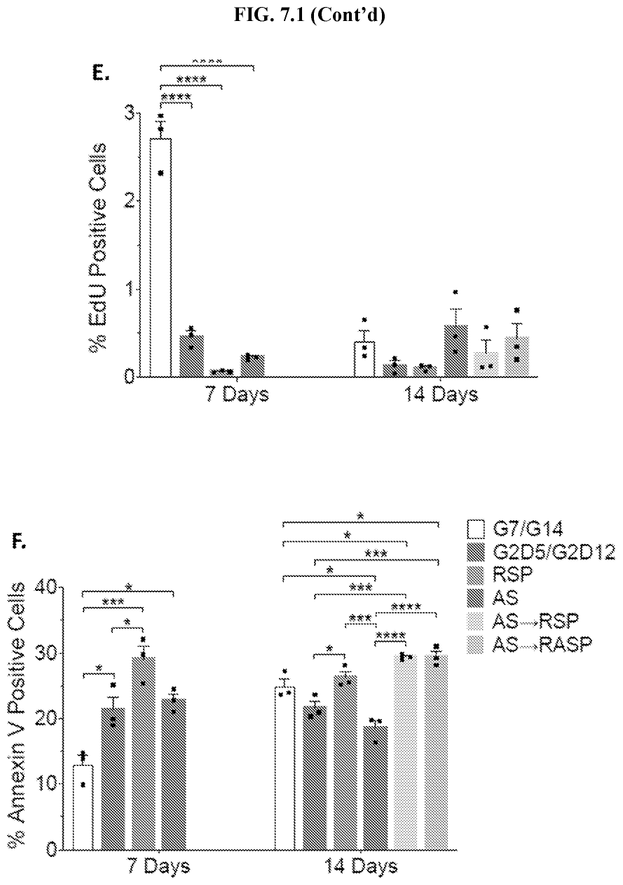

Furthermore, exposure to AS for the entire differentiation protocol, i.e., AS and AS→RASP, decreased expression of MUC2 and ALP1 and increased expression of LGR5 compared to those that received RSP alone, for any amount of time (FIG. 7.1A). In terms of other lineages, immunofluorescence staining showed that the enterocyte marker cytokeratin 20 (CK20) was expressed in all differentiation conditions, but with very little stain noted in AS→RASP (FIG. 7.1B). MUC2 staining was only noted in RSP, AS and AS→RSP enteroids while LYZ, similar to CK20, was present in all differentiation conditions except AS→RASP (FIG. 7.1C and FIG. 7.1D).

The effect of the disclosed differentiation protocols on proliferation and apoptosis after 7 and 14 days of culture was evaluated. As expected, at 7 days GM supported cell proliferation, as evidenced by a significantly higher fraction of EdU-positive (EdU+) cells (2.7%), after a two-hour pulse, compared to all other conditions. There were no significant differences between DM, RSP, and AS, which ranged from 0.1%-0.5% EdU+ cells. At the end of the experiment (day 14), there were no differences in the percentage of EdU+ cells between any groups (which ranged from 0.1-0.6%), suggesting that GM conditions are not able to sustain ongoing proliferation indefinitely (FIG. 7.1E). Analysis of apoptosis at 7 days showed GM had the lowest percentage of Annexin V-positive cells (3%) compared to each differentiation condition, with RSP showing the highest fraction at 29%. At day 14, despite modest differences between groups, levels of apoptosis were fairly consistent ranging from 19%-29% (FIG. 7.1F). Further, enteroids exposed to RSP (including RSP, AS→RSP, and AS→RASP) appeared to have more cells in apoptosis compared to DM and AS.

All four differentiation strategies induced a high fraction of CHGA+ enteroids, ranging from 79% to 88% (RSP, 79%; AS, 82%; AS→RSP, 81%; AS→RASP, 88%), as assessed by immunostaining (FIG. 5B and FIG. 5C). Quantitative flow cytometric analysis revealed 3.6% of all cells exposed to AS→RSP to be CHGA+, which is between the 1.0% seen in RSP alone and the 6.0% seen in AS alone. AS→RASP had the highest percentage of CHGA+ cells at 7.0%, more than 100 times the number seen with G2D12 alone (FIG. 5D). Taken together, these data revealed that exposure of human enteroids to AS→RASP was the most effective way to induce EE cell differentiation, in terms of expression of CHGA, PAX4, NEUROD1, and NEUROG3 as well as overall number of CHGA+ cells.

### Example 6: Hormone Production and Secretion Mirror Gene Expression Changes in Enteroids

To assess hormone production and secretion during EE cell differentiation, duodenal-associated hormones were assayed in response to the various differentiation conditions. Immunofluorescent staining showed that SST was expressed similarly in all differentiation conditions, aside from G2D12, with 42% to 53% of enteroids containing SST-positive cells (RSP, 45%; AS, 42%; AS→RSP, 53%; AS→RASP, 51%) (FIGS. 6A and 6B). 5HT was expressed in a higher percentage of enteroids treated with AS throughout the entire differentiation period than those that only received RSP (RSP, 44%; AS, 75%; AS→RSP, 59%; AS→RASP, 76%) (FIGS. 6C and 6D). Further, an induction in GIP-positive enteroids was detected in response to all differentiation conditions, with the exceptions of G2D12 and AS→RASP (RSP, 66%; AS, 53%; AS→RSP, 56%; AS→RASP, 10%) (FIGS. 6E and 6F). Further, CCK-positive enteroids were predominantly detected in response to RSP (29%) and AS→RASP (30%) compared to all other treatment groups (G2D12, 0%; AS, 4.5%; AS→RASP, 2.6%) (FIG. 6G, FIG. 6H).

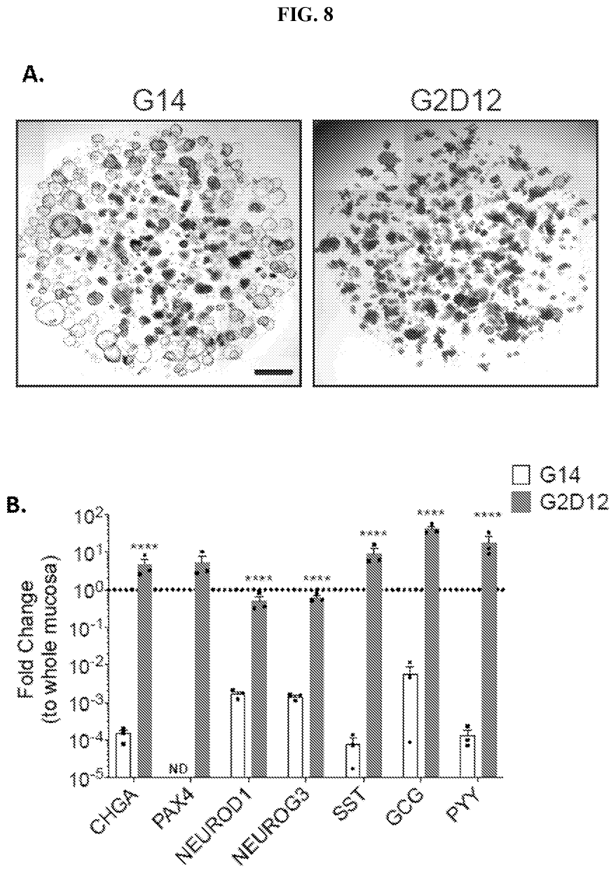

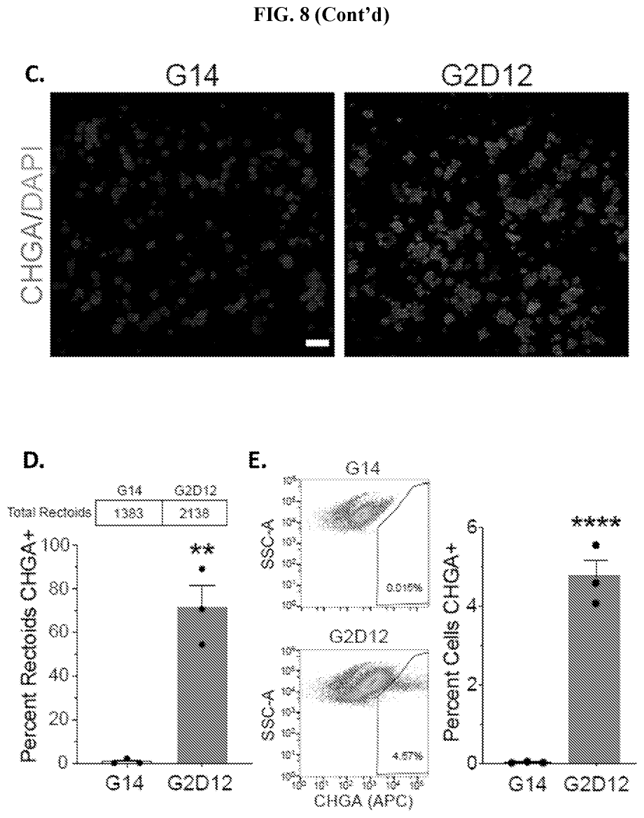

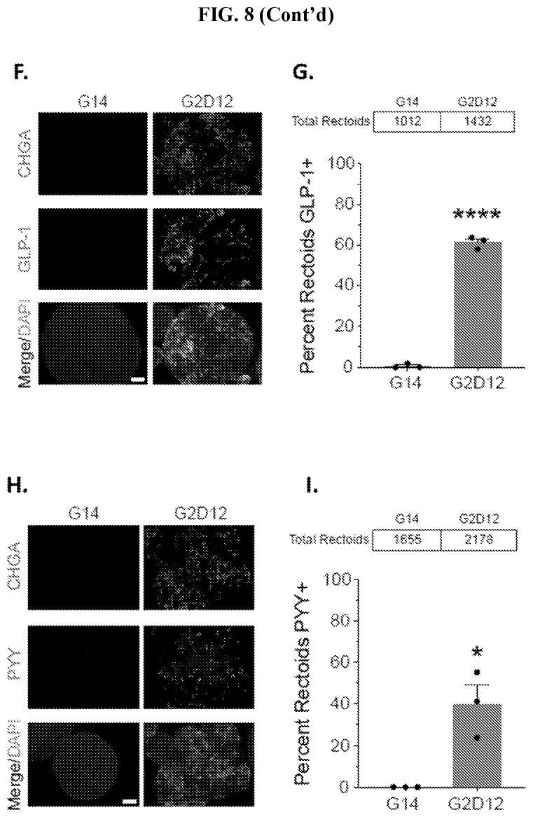

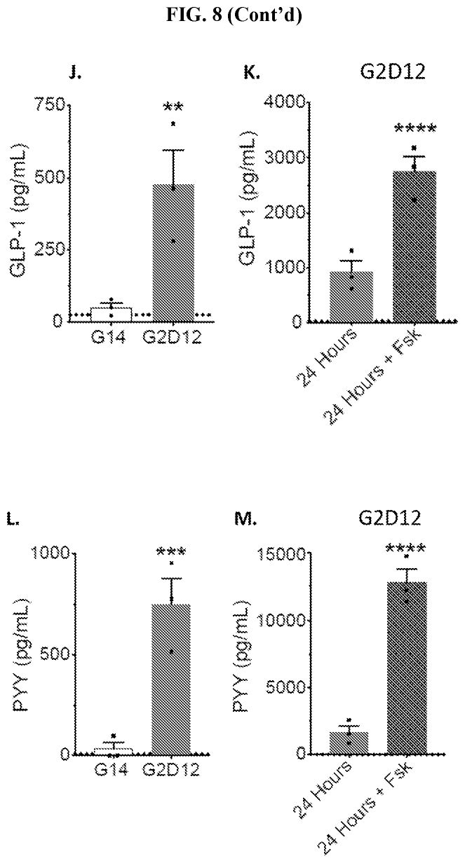

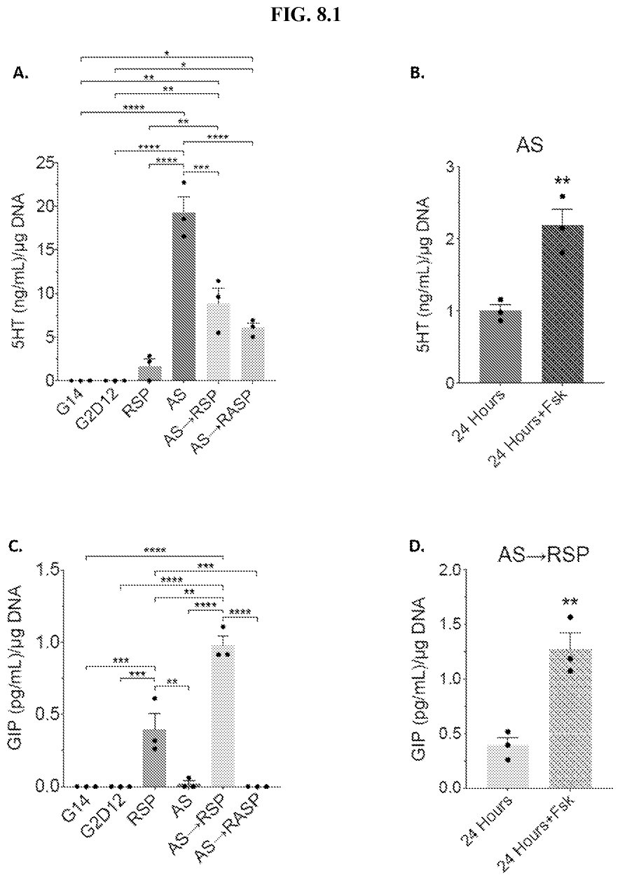

Finally, to assess hormone secretion, conditioned media from each differentiation condition disclosed here were assayed. Exposure to AS alone induced higher levels of 5HT secretion than all other conditions, with AS→RSP conditioned media showing higher levels of 5HT when compared to RSP and AS→RASP (FIG. 7A). Importantly, secretion of 5-HT was significantly increased when differentiated enteroids were exposed to the adenylate cyclase activator forskolin (Fsk) (FIG. 7B). These patterns persisted when corrected for total DNA content, with the exception of a modest difference between AS→RSP and AS→RASP (FIG. 8.1A and FIG. 8.1B). Secretion of GIP was highest following exposure to AS→RSP, compared to RSP alone, while AS and AS→RASP treated enteroids revealed no secretion of GIP (FIG. 7C; FIG. 8.1C). Secretion of GIP from enteroids exposed to AS→RSP was also significantly increased after exposure to Fsk (FIG. 7D and FIG. 8.1D). Overall, these data show that RSP and AS, either alone or in combination, can induce protein expression and secretion of multiple duodenal hormones (5HT, GIP, SST, and CCK) from human enteroids, with specific differences depending on exposure and/or timing of RSP and AS treatment.

Studies for establishing a role for Wnt, Notch, MAPK, and BMP signaling in EE lineage differentiation have been performed using the enteroid culturing system, which approximates in vivo growth and development due to its 3D nature. However, there are significant shortcomings to the analyses of intestinal enteroid differentiation. First, RNA expression does not always mirror protein expression, as evidenced by the presence of GIP mRNA in AS→RASP enteroids (FIG. 5A) with little protein expression noted on immunofluorescence (FIGS. 6E and 6F) and no secreted protein seen on ELISA (FIG. 7C). Further, enteroids grown in G14 were not appropriate as the sole reference in differentiation experiments. For example, G2D12, but not G14, treatment induced expression of CHGA mRNA (FIG. 1B), but the comparison of enteroid mRNA expression levels to whole duodenal mucosa revealed that G2D12 alone induces only a very low level of CHGA compared to native tissue. Moreover, immunodetection of CHGA expression revealed significant heterogeneity between individual enteroids, even when cultured under the same experimental conditions, as evidenced by only a small minority of enteroids and cells staining CHGA+ in G2D12 and the variability in hormone expression using immunofluorescence. These results highlight the limitations of conventional human EE differentiation protocols.

### Example 7: Differentiation Media Induces EE Cell Differentiation in Human Rectoids

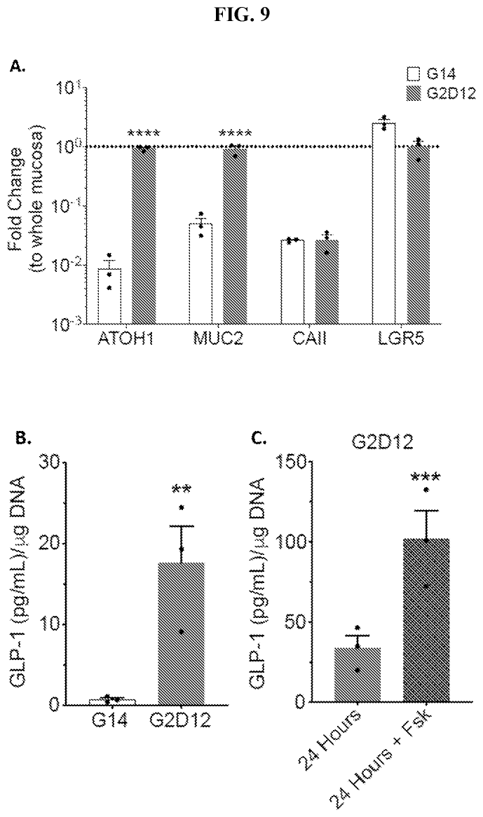

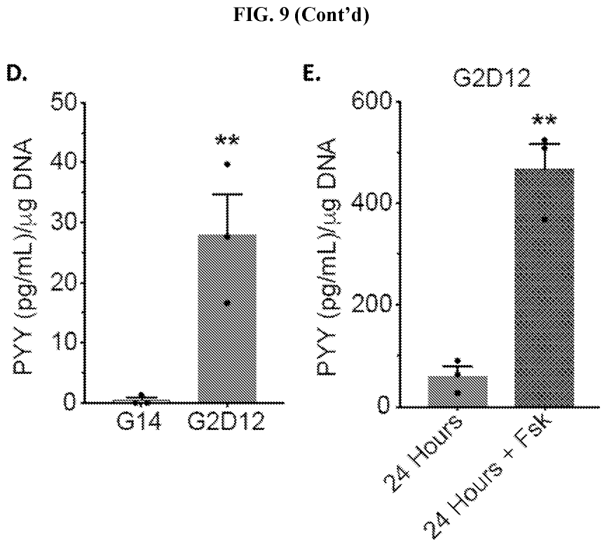

In comparison to enteroids, rectoids showed a much stronger induction of EE cells when exposed to G2D12. To evaluate the ability of the disclosed DM to induce EE cell differentiation outside of the duodenum, organoids derived from rectal ISCs (rectoids) were exposed to the G14 and G2D12 protocols. Rectoids treated with G14 maintained spherical structures, while those exposed to G2D12 had a mixture of spherical and budding structures (FIG. 8A). In contrast to enteroids, rectoids exposed to G2D12 had significantly increased expression of the majority of EE markers, including CHGA, PAX4, NEUROD1, NEUROG3, SST, GCG, and PYY, many of which were at or above the expression level observed in whole rectal mucosa (FIG. 8B). Rectoids exposed to G2D12 also had significantly higher expression of ATOH1 and MUC2, but no change in LGR5 or CAII expression, a marker for absorptive cells of the rectum (Bekku, S. et al. Res Exp Med (Berl) 198, 175-185, doi:10.1007/s004330050101 (1998); Gonzalez, L. M., Williamson, I., Piedrahita, J. A., Blikslager, A. T. & Magness, S. T. PLoS One 8, e66465, 20 doi:10.1371/journal.pone.0066465 (2013))^{58,59}, when compared to G14 (FIG. 9A).

Immunofluorescence staining revealed CHGA+ cells within a majority of G2D12 rectoids (72%) (FIG. 8C and FIG. 8D) and quantitative flow cytometric analysis revealed 4.7% of all cells exposed to G2D12 to be CHGA+(FIG. 8E). Additional immunofluorescence staining revealed that both GLP-1 and PYY were expressed in a large number of G2D12 rectoids (GLP-1, 61%; PYY, 40%) (FIGS. 8F-8I). Conditioned media from G14 and G2D12 rectoids was assayed to evaluate for hormone secretion and found significant levels of both GLP-1 and PYY in G2D12 rectoids compared to G14, which was increased by stimulation with Fsk (FIGS. 8J-8M; FIGS. 9B-9E). Overall, these data suggested that G2D12 is sufficient to induce EE cell differentiation of rectal ISCs in the rectoid culture model.

### Example 8: Isolation of Human Intestinal Crypts

Tissues were procured as previously described (Kasendra M, Tovaglieri A, Sontheimer-Phelps A, et al. Sci Rep 2018; 8:2871)³¹. In short, de-identified endoscopic biopsies were collected from grossly unaffected tissues in pediatric patients undergoing esophagogastroduodenoscopy at Boston Children's Hospital for gastrointestinal complaints and used for organoid derivation e.g., duodenal enteroid derivation. Further, de-identified duodenal resections were collected from adult patients undergoing pancreaticoduodenectomy at Massachusetts General Hospital prior to chemotherapy or radiation therapy forpancreatic carcinoma. Whole mucosal biopsies were generated from the duodenal resections using endoscopic biopsy forceps and used fortotal RNA isolation. Age and sex ofdonors can be found in TABLE 2, but were unknown to researchers when experiments were being pelformed. Only macroscopically normal-appearing tissue was used from patients without a known gastrointestinal diagnosis. Each experiment was performed on at least three independent organoid (e.g., enteroid) lines derived from pediatric biopsy samples. Informed consent and developmentally appropriate assent were obtained at Boston Children's Hospital from the donors' guardian and the donor, respectively. Informed consent was obtained at Massachusetts General Hospital from the donors. All methods were approved and carried out in accordance with the Institutional Review Boards of Boston Children's Hospital (Protocol number IRB-P00000529) and Massachusetts General Hospital (Protocol number IRB-2003P001289).

Resections were briefly washed with pre-warmed DMEM/F12, after which the epithelial layer was separated from the rest of the duodenum manually with sterilized surgical tools then taken for RNA isolation. To isolate crypts, pediatric biopsies were digested in 2 mg/mL of Collagenase Type I (Life Technologies, 17018029) reconstituted in Hank's Balanced Salt Solution for 40 minutes at 37° C. Samples were then agitated by pipetting followed by centrifugation at 500×g for 5 minutes at 4° C. The supermatant was then removed, and crypts resuspended in 200-300 μL of Matrigel® (Corning, 356231), with 50 μL being plated onto 4-6 wells of a 24-well plate and polymerized at 37° C.

TABLE 2
Description of samples

| Identification | Age | Sex | Application |
|---|---|---|---|
| H357 | 13 years | Male | Enteroid line |
| H367 | 18 years | Male | Rectoid line |
| H368 | 14 years | Female | Enteroid line |
| H389 | 14 years | Male | Enteroid line |
| H393 | 15 years | Female | Enteroid line |
| H395 | 18 years | Male | Enteroid line |
| H407 | 13 years | Male | Enteroid line |
| H416 | 21 years | Female | Enteroid line |
| H439 | 19 years | Female | Enteroid line |
| H522 | 82 years | Female | Duodenal mucosa RNA |
| H544 | 55 years | Female | Duodenal mucosa RNA |
| H545 | 70 years | Male | Duodenal mucosa RNA |
| H567 | 15 years | Male | Rectoid line |
| H587 | 17 years | Female | Rectoid line |
| H598 | 18 years | Female | Rectal mucosa RNA |
| H609 | 17 years | Female | Rectoid line |
| H616 | 12 years | Female | Rectoid line |
| H620 | 17 years | Female | Rectal mucosa RNA |
| H637 | 15 years | Female | Rectal mucosa RNA |
| H642 | 15 years | Female | Rectoid line |
| H645 | 15 years | Female | Rectoid line |
| H646 | 17 years | Female | Rectoid line |

### Example 9: Culturing of Human Duodenal Enteroids In Vitro

Isolated crypts in Matrigel® were grown in growth media (GM) (TABLE 1A and TABLE 1B) and the resulting enteroids were passaged every 6-8 days as needed, with media changes occurring every two days. To passage, Matrigel® was mechanically dissociated from the well and resuspended in 500 μL of Cell Recovery solution (Corning, 354253) for 40-60 minutes at 4° C. To aid in separating the Matrigel® and enteroids, the tubes are gently inverted and then centrifuged at 500×g for 5 minutes at 4° C. The supernatant was then removed, and organoids (e.g., enteroids) resuspended in Matrigel®, followed by mechanical disruption via a bent-tipped pipette. Organoids were passaged at a 1:2 dilution, with 50 μL per well of a 24-well plate. After plating, the organoids were incubated at 37° C. for 10 minutes to allow the Matrigel® to set. Once complete, 500 μL of GM was added to each well.

For differentiation, enteroids were passaged and grown in GM for two days, to allow for ISC expansion, after which the organoids were transitioned to tissue-specific differentiation media (DM) with additional small molecules added as described (TABLE 1A and TABLE 1B). Media was changed every two days, with Tubastatin A being removed after the second day of differentiation. Enteroids and rectoids were taken for analysis after 14 days.

### Example 10: Gene Expression Analysis

Total RNA was purified from individual wells using TRI®Reagent (Sigma) and the Direct-zol™ RNA kit (Zymo Research), following the manufacturer's protocol. RNA concentration was determined using a NanoDrop™ 1000 spectrophotometer (Life Technologies). RNA was treated with DNAse (Promega) and reverse transcribed using the High-Capacity cDNA Reverse Transcription Kit (Life Technologies). Gene expression analysis was then performed by Real Time quantitative PCR (qPCR) using a QuantStudio 6 Flex thermocycler (Life Technologies). Taqman primers from Life Technologies (TABLE 3) were used. 18S transcripts were used as the internal control and data were expressed using the 2^{-ddCt} method with Ct limit of 40. Fold change, unless otherwise stated, was compared to total RNA derived from adult whole mucosal tissue biopsies.

TABLE 3
List of Taqman qPCR Primers from Life
Technologies (Cat. # 4331182)

| Name | Abbreviation | Identifier |
|---|---|---|
| 18S | 18S | Hs99999901_s1 |
| Intestinal alkaline phosphatase | ALPI | Hs00357579_g1 |
| Atonal bHLH transcription factor 1 | ATOH1 | Hs00944192_s1 |
| Carbonic anhydrase II | CAII | Hs01070108_m1 |
| Cholecystokinin | CCK | Hs00174937_m1 |
| Chromogranin A | CHGA | Hs00900370_m1 |
| GATA binding protein 4 | GATA4 | Hs00171403_m1 |
| Glucose-dependent insulinotropic | GIP | Hs00175030_m1 |
| polypeptide | | |
| Leucine-rich repeat-containing | LGR5 | Hs00969422_m1 |
| G-protein coupled receptor 5 | | |
| Lysozyme | LYZ | Hs00426232_m1 |
| Mucin 2 | MUC2 | Hs03005103_g1 |
| Neuronal differentiation 1 | NEUROD1 | Hs01922995_s1 |
| Neurogenin 3 | NEUROG3 | Hs01875204_s1 |
| Paired box 4 | PAX4 | Hs00173014_m1 |
| Pancreatic and duodenal homeobox 1 | PDX1 | Hs00236830_m1 |
| Peptide YY | PYY | Hs00373890_g1 |
| Somatostatin | SST | Hs00356144_m1 |

### Example 11: DNA Isolation

To isolate organoid genomic DNA, 200 μL of 50 mM NaOH was added to a single well of a 24-well plate and the Matrigel® was mechanically dissociated. The samples were then transferred to 1.5 mL microcentrifuge tubes and placed in a 95° C. heat block for 20 minutes. The tubes were then vortexed, after which 25 μL of 1M Tris-HCl was added. The samples were then centrifuged at 14,000 rpm for 10 minutes. The DNA content of the supermatant was then assayed using a NanoDrop™1000 spectrophotometer (Life Technologies).

### Example 12: Immunofluorescence

Immunofluorescence staining was performed as previously described with minor modifications (Dekkers J F, Alieva M, Wellens L M, et al. Nat Protoc 2019; 14:1l756-1771)³². Organoids were grown in and isolated from Matrigel® as noted above. 1-3 wells from a 24-well plate were washed in 200 μL of 1× phosphate-buffered saline (PBS) and moved in suspension to a 1.5 mL microcentrifuge tube. Each tube was centrifuged at 800×g for 5 minutes at 4° C. to pellet organoids. PBS was aspirated, and organoids were fixed in 200 μL of 4% paraformaldehyde (PFA) for 20 minutes on ice, shaking. Each tube was centrifuged again as above, and PFA was aspirated. The organoids were then resuspended in 500 μL of 0.3% Triton-X in PBS and moved to a 48-well plate for the remaining steps. The organoids were permeabilized for 30 minutes at room temperature, shaking. Between each step, organoids were allowed to settle to the bottom of each well, the plate was angled, and the solution aspirated by careful pipetting. Organoids were then blocked with 5% bovine serum albumin in PBS for 1.5 hours at room temperature, shaking. This was followed by three five-minute washes in 500 μL of PBS at room temperature, shaking.

Each well was then incubated in 200 μL of primary antibodies diluted in 5% BSA/0.1% Trition-X in PBS at 4° C. overnight. This was followed by five washes in 500 μL of 0.1% Triton-X in PBS for 15 minutes each at room temperature, shaking. 200 μL of secondary antibodies, including Alexa Fluor 488- or 647-conjugated anti-mouse (Invitrogen, A-21202, or A-31571, 1:400), Alexa Fluor 488-conjugated anti-rat (Invitrogen, A-21208, 1:400), Alexa Fluor 488-conjugated anti-goat (Invitrogen, A-11055, 1:400) or Alexa Fluor 647-conjugated anti-rabbit (Invitrogen, A-31573, 1:400), diluted in 0.1% Triton-X in PBS, which were then added to each well and incubated for two hours at room temperature, shaking. Organoids were then washed as above, then moved to new 1.5 mL centrifuge tubes. During the last wash, 4′,6-diamidino-2-phenylindole (DAPI, Life Technologies, D1306) was added at a concentration of 1:1000 for nuclear staining. Organoids were then centrifuged at 1000×g for 5 minutes at 4° C. to assist in removal of as much PBS as possible. Slides were prepared by drawing three circles with a hydrophobic pen (Vector Laboratories, H-4000). Enteroids were then resuspended in 20 μL of Prolong Gold Antifade mountant (Life Technologies, P36930), and droplets placed within hydrophobic circles. The organoids were spread out to reduce clumping, sealed with a coverslip, and allowed to dry overnight at room temperature. Slides were stored at 4° C. for future imaging. Images were acquired using a Nikon upright Eclipse 90i microscope with a 20×/0.75 Plan-Apochromat objective and adjusted for brightness and contrast in Fiji (Schindelin J, Arganda-Carreras I, Frise E, et al. Nat Methods 2012; 9:676-82)⁶⁸.

Primary antibodies included Chromogranin A (CHGA) (Agilent/Dako, M086901-2, 1:100; Millipore Sigma, HPA017369, 1:100), Serotonin (Abcam, ab66047, 1:100), GIP (Invitrogen, PA5-76867, 1:100) and Somatostatin (R&D Systems, mab2358, 1:100).

### Example 13: Quantification of Immunofluorescent Enteroids

This technique was adapted from a previously described method (Borten M A, Bajikar S S, Sasaki N, et al. Sci Rep 2018; 8:5319)⁶⁹. Immunofluorescent images were acquired using an Invitrogen EVOS FL 2 Auto microscope (Life Technologies). Representative images of stained enteroids were taken at 2× magnification. The stitched images were then processed in Fiji (Schindelin J, Arganda-Carreras I, Frise E, et al. Nat Methods 2012; 9:676-82)⁶⁸. The color of the DAPI images was converted to 8-bit grayscale and then the image was smoothed by applying a Gaussian Blur filter (radius=4, scaled units). Thresholding of the smoothed images was performed using manual adjustment to achieve optimal separation of individual enteroids. Watershed and Find Edges filters were then applied to segment any clumped enteroids. Post-segmentation analysis was performed to outline and count individual enteroids using Analyze Particles (size=4,000-100,000, circularity=0.30-1.00, exclude on edges). Each image was then manually curated to exclude debris and enteroids exhibiting background fluorescence. Any remaining clumped enteroids were manually separated prior to quantification. The outlines generated from the DAPI images were then applied to the corresponding images from the other fluorescent channels. Each color image was converted to 8-bit grayscale and then the HiLo Lookup Table was applied. The threshold gate for stained cells was found by manual adjustment of positively stained enteroids to achieve optimal representation. The threshold gate for each channel was then applied to each experimental condition. The Mean metric was extracted with ROI manager (measure) and compiled for analysis. Enteroids with a Mean value of more than zero were considered positive.

### Example 14: Flow Cytometry

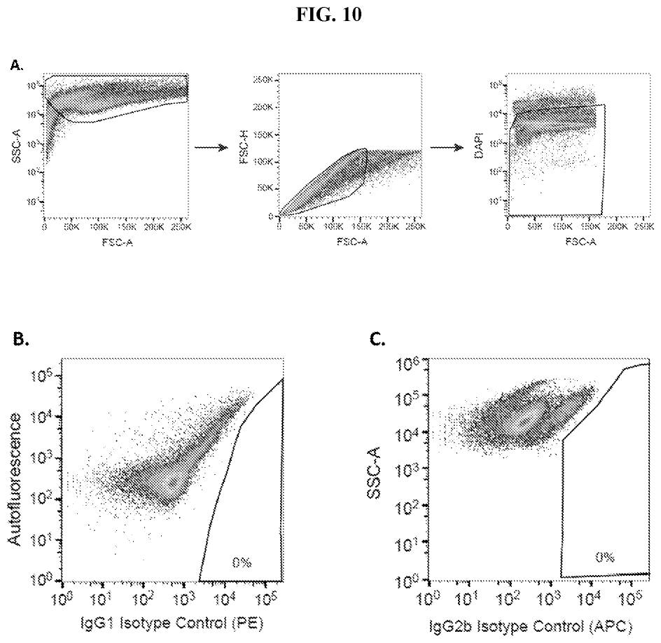

Organoids were incubated in Cell Recovery solution for 40-60 minutes at 4° C. to remove the Matrigel® and then centrifuged at 500×g for 5 minutes at 4° C. To achieve single cell suspension, organoids were then incubated in 500 μL of TrypLE™ Express (Thermo Fisher Scientific) at 37° C. for 30 minutes and broken up by repeated pipetting using a bent P1000 pipette tip. Each sample was then diluted in 800 μL of 20% FBS in Advanced DMEM/F12 and then centrifuged at 800×g for 5 minutes at 4° C. To mark dead cells, each sample was then incubated in DAPI (1:1000) diluted in 2% FBS/2 mM EDTA/calcium-free DMEM for 20 minutes at room temperature, then centrifuged at 800×g for 5 minutes at 4° C. and washed in 2% FBS/2 mM EDTA/DMEM. Cells were then incubated in 1% PFA for 15 minutes at room temperature, washed with 2% FBS/PBS and then permeabilized in 0.2% Tween 20 in 2% FBS/PBS for 15 minutes at 37° C. Following centrifugation, cells were resuspended in 0.1% Tween 20/2% FBS/2 mM EDTA in PBS with PE/Alexa Fluor 647-conjugated CHGA (BD Biosciences, 564563, 1:100) or PE/Alexa Fluor 647-conjugated mouse IgG1, K isotype, (BD Biosciences, 554680, 1:200) or with no antibody (the latter two acting as controls) for 30 minutes on ice. Cells were then washed in 0.1% Tween 20/2% FBS/2 mM EDTA in PBS, filtered through a 37-micron mesh, and then analyzed on a BD FACSAria II SORP flow cytometer. The gating strategy was performed as described here (FIG. 10). Enteroid cells were differentiated from cellular debris based on their forward and side scatter area (FSC-A and SSC-A, respectively) parameters. Cells were then examined based on their FSC-A and FSC-Height (H) to exclude doublets. 4′,6-diamidino-2-phenylindole (DAPI) staining is then utilized to identify dead cells, with DAPI high-positive cells being excluded from further gating. The CHGA-positive gate was set by using an IgG1 K isotype control conjugate with phycoerythrin (PE).

### Example 15: Cell Proliferation

Enteroid cell proliferation was assessed using the Click-iT™ EdU Alexa Fluor™ 488 Flow Cytometry Assay kit (Thermo Fisher Scientific) following the manufacturer's protocols. Briefly, enteroids were grown in Matrigel® as noted above and taken for analysis after 7 and 14 days. Enteroids were labeled following incubation with 10 μM of EdU at 37° C. for 2 hours and then incubated with TrypLE™ Express as above to isolate single cells. All samples were then diluted in 800 μL of 20% FBS in Advanced DMEM/F12 and then centrifuged at 800×g for 5 minutes at 4° C. Fixation, permeabilization, and the Click-iT reaction were performed in accordance with manufacturer's protocols before being filtered through a 37-micron mesh, and then analyzed on a BD FACSAria II SORP flow cytometer.

### Example 16: Cell Apoptosis

Apoptosis was assessed using the Dead Cell Apoptosis Kit with Annexin V Alexa Fluor™ 488 & Propidium Iodide (Thermo Fisher Scientific) following the manufacturer's protocols. For these experiments, enteroids were grown in Matrigel® as noted above and taken for analysis after 7 and 14 days. To isolate single cells, enteroids were incubated with TrypLE™ Express as above. All samples were then diluted in 800 μL of 20% FBS in Advanced DMEM/F12 and then centrifuged at 800×g for 5 minutes at 4° C. Samples were then incubated with propidium iodide and annexin V per manufacturer's protocol. Cells were filtered through a 37-micron mesh, and then analyzed on a BD FACSAria II SORP flow cytometer.

### Example 17: ELISA

Hormone quantification for GIP (MilliporeSigma, EZHGIP-54K), GLP-1, PYY, and serotonin (Eagle Biosciences/DLD Diagnostika GmbH, SER39-KO1) and were performed using kits and following manufacturer's protocols. For these experiments, organoids were grown in 48-well plates to aid in concentrating the hormone of interest. Conditioned media was taken on day 14 of differentiation. For GIP quantification, Diprotin A (Tocris, 6019, 100 μM), a dipeptidyl peptidase 4 inhibitor, was added daily to the media for the last two days of differentiation to prevent GIP and GLP-1 degradation. For hormone induction studies, forskolin (Fsk) (10 μM) was added to fresh media 24-hours prior to conditioned media being taken. As an additional control, differentiation media not exposed to cells was also evaluated. This value was then subtracted from each experimental sample.

### Example 18: Sample Preparation for Single Cell RNA Sequencing

Enteroids were incubated in Cell Recovery solution for 40-60 minutes at 4° C. to remove the Matrigel® and then centrifuged at 500×g for 5 minutes at 4° C. To achieve single cell suspension, organoids were then incubated in 500 μL of TrypLE™ Express at 37° C. for 30 minutes and broken up by repeated pipetting using a bent P1000 pipette tip. Each sample was then diluted in 800 μL of 20% FBS in Advanced DMEM/F12, filtered through 37 μm mesh, and then centrifuged at 300×g for 10 minutes at 4° C. Dead cells were then removed using magnetic-activated cell sorting (MACS) separation following the manufacturer's protocol. Briefly, each sample was resuspended in 100 μL Dead Cell Removal MicroBeads (Miltenyi Biotec) and incubated at room temperature for 15 minutes. 400 μL of 1×Binding Buffer was then added to each sample, after which the samples were applied to prepared MACS columns placed in a MACS Separator. Flow-through containing live cells was collected. This was followed by washing the columns four times with 1× Binding Buffer, which were combined with the initial flow-through. These samples were then centrifuged at 800×g for 5 minutes at 4° C.

### Example 19: Hashtag-Labeling of Sequencing Samples

Single cells isolated after dead cell removal (see, Example 18) were labeled prior to sequencing with Totalseq™-B hashtag antibodies (Biolegend), targeted against CD298 and β2-microglobulin, per the manufacturer's protocol⁴⁷. Briefly, each of the nine samples (three separate differentiation conditions in three enteroid lines) were resuspended in 50 μL of Cell Staining Buffer (Biolegend) and blocked with 5 pL of Human TruStain FcX^{T}M blocking reagent (Biolegend) for 10 minutes at 4° C. The supernatant was then removed, and each sample resuspended in 50 μL of Cell Staining Buffer containing 1 pg of a unique Totalseq™-B hashtag antibody. Samples were incubated for 30 minutes at 4° C. and then washed three times in 3.5 mL of Cell Staining Buffer. The samples were then filtered through a 37 μm mesh, resuspended in 2% BSA in PBS at a concentration of 1500 cells/μL, and then pooled together. Of note, prior to the scRNA-seq experiment, antibody binding was verified using fluorophore-conjugated antibodies with the same binding specificity.

### Example 20: Droplet-Based Single Cell RNA Sequencing and Alignment

A total of 180,000 cells as one pool were inputted across four channels, with a recovery rate of approximately 30,000 cells across all four channels. RNA libraries were prepared from single cells by the Single Cell Core at Harvard Medical School, Boston, Mass., using the 10× Chromium Single Cell 3′ Library Chip (V3.1) with dual indexed barcodes. Briefly, single cells were partitioned into Gel Beads in Emulsion (GEMs) by the Chromium Controller. Prior to partitioning, hashed cells were pooled together and “super-loaded” to reduce batch effects across samples (Stoeckius, M. et al. Genome Biol 19, 224, doi:10.1186/s13059-018-1603-1 (2018)⁴⁷. Once partitioned, cells were lysed and RNA was captured, reverse transcribed, and amplified. cDNA was then enzymatically fragmented and sample indices were attached to both ends of the fragment. Following library prep, the transcriptomes were sequenced by the Molecular Biology Core Facilities at the Dana-Farber Cancer Institute on an Illumina NovaSeq 6000 sequencing platform. The pooled sample was split across two lanes on a NovaSeq S1 flow cell.

After sequencing, the files were processed, demultiplexed and aligned using 10× 's Cell Ranger software (v5.0.1). Briefly, raw Binary Base Call (BCL) files were demultiplexed using the cellranger mkfastq function, a wrapper of Illumina's bcl2fastq, with the filter dual index argument set to true. Following the generation of the FASTQ files, the samples were aligned to the hg19 human genome and features were counted using the cellranger count function. This function aligned the Unique Molecular Identifiers (UMIs) of each individual cell to the related gene and counted the number of detected UMIs to generate a feature-barcode matrix. In addition, it also measures the hashtag oligo counts for each individual cell from the relevant hashtag antibodies that were used to label each condition. In addition, for analysis of RNA velocity, both un-spliced and spliced RNA variants were aligned and counted using the velocyto.py package (v0.17.16) (La Manno, G. et al. Nature 560, 494-498, doi:10.1038/s41586-018-0414-6 (2018))⁵.

### Example 21: Single Cell RNA Sequencing Analysis

Analysis of the scRNA-seq data was conducted using the Seurat package (v4.0.1), within the R programming language (v4.0.3). Initially, cells were filtered to only include those with at least one detectable hashtag antibody signal, and then hashtag counts were normalized, and the cell samples were demultiplexed using the HTOdemux function built into Seurat. This function ranked a cell as being positive for a specific hashtag signal, if the detected signal was in the 99th percentile or higher. Cells were then demultiplexed, with cells that had no positive signals being labeled as negative cells, cells with only one positive signal being labeled by their respective positive signal, and cells with two or more positive signals being labeled as doublets. Following demultiplexing, the dataset was pre-processed to remove cells that were deemed of lower quality, based on their number of unique genes (<200 unique genes) and the proportion of UMIs that corresponded to mitochondrial RNA (>25% of UMIs) (FIG. 4.1A) (Stuart, T. et al. Cell 177, 1888-1902 e1821, doi:10.1016/j.cell.2019.05.031 (2019)⁴⁸.

After the removal of doublet barcodes, negative barcodes and the remaining barcodes that were deemed to be of lower quality, a resulting combined dataset of 14,767 cells was used for further analysis. To better control for batch effects and to improve the ability to detect diversity within the samples, the gene counts were normalized using regularized negative binomial regression, using SCTransform (Hafemeister, C. & Satija, R. Genome Biol 20, 296, doi:10.1186/s13059-019-1874-1 (2019))⁷⁰. Following normalization, dimensional reduction was performed by running Principal Component Analysis (PCA) and, based on the elbow plot generated, selected the first 20 principal components for defining the K nearest neighbors and the Uniform Manifold Approximation and Projection (UMAP) plot. After dimensional reduction, 15 clusters were defined using Louvain clustering (resolution=0.5). Differentially expressed genes were identified across each cluster using the Wilcoxon rank sum test via the FindAllMarkers function built into Seurat. Only genes that were expressed in at least 25% of cells and had a log-fold change of 0.25 were considered in this analysis. These differential genes, as well as classical epithelial markers, were used to broadly categorize the clusters into six epithelial cell types and include LGR5, AXIN2 and ASCL2 for intestinal stem cells, KRT20, FABP2, and FABP1 for enterocytes, MUC2, FCGBP, and GFI1 for goblet cells, CHGA, NEUROG3, NEUROD1 and PAX4 for enteroendocrine cells, CD44 for progenitor cells and MKI67 for proliferating cells (Barker, N. Nat Rev Mol Cell Biol 15, 19-33, doi: 10.1038/nrm3721 (2014); van der Flier, L. G. & Clevers, H. Annu Rev Physiol 71, 241-260, doi:10.1146/annurev.physiol.010908.163145 (2009); Noah, T. K., Donahue, B. & Shroyer, N. F. Exp Cell Res 317, 2702-2710, doi:10.1016/j.yexcr.2011.09.006 (2011))⁵¹⁻⁵³.

Finally, enteroid EE cell identity was compared to the gene set signature from tissue EE cells isolated from the murine small intestine (Haber, A. L. et al. Nature 551, 333-339, doi: 10.1038/nature24489 (2017))⁴⁹. To compare the gene signature, the gene module score of each individual cell was calculated using the AddModuleScore function found in Seurat v3 and v4, which calculates the score based on the enrichment of the specified gene set compared to randomly selected genes with a comparable average gene expression (Stuart, T. et al. Cell 177, 1888-1902 e1821, doi:10.1016/j.cell.2019.05.031 (2019)⁴⁸. The AddModuleScore function calculated a scaled score between 0 to 1.

### Example 22: Trajectory Analysis with scVelo

RNA velocity was calculated using the scVelo package (v0.2.3) with Scanpy (v1.7.1) on Python (v3.7.9) (Bergen, V., Lange, M., Peidli, S., Wolf, F. A. & Theis, F. J. Nat Biotechnol 38, 1408-1414, doi:10.1038/s41587-020-0591-3 (2020)⁵⁴. To perform trajectory analysis of epithelial organoids, the un-spliced and spliced variant count matrix that was previously calculated using velocyto was fused with an anndata object containing the UMAP information and cluster identities defined in Seurat analysis. The combined dataset was then processed using the scVelo pipeline: The ratio of un-spliced:spliced RNA for each gene was normalized and filtered using the default settings. Afterwards, the first and second moments were calculated for velocity estimation. Following moment calculation, the dynamic model was used to calculate the RNA velocities. The dynamic model iteratively estimated the parameters that best model the phase trajectory of each gene, thereby capturing the most accurate, albeit more computationally intensive, estimate of the dynamics for each gene. These approaches were used to graphically model the RNA velocity for each culture condition.

### Example 23: Quantification and Statistical Analysis

All experiments were repeated using at least three different human organoid lines and representative data from a single line is shown. For qPCR and flow cytometry studies, each condition was performed using pooled enteroids from 3-5 wells, unless otherwise noted, with each well acting as a single sample. Whole mucosal biopsies from duodenal resections and whole mucosa rectal biopsies were combined from three different individuals to generate a single reference sample for all enteroid and rectoid experiments.

Prior to statistical analysis, all qPCR data were transformed using log₁₀. When analyzing only two conditions, statistical significance was determined by unpaired, two-tailed Student's t-test, combined with the Holm-Sidak method to control for multiple comparisons. When analyzing more than two conditions, statistical significance was determined by either one-way or two-way Analysis Of Variance (ANOVA), followed by Tukey post hoc analysis. Specific conditions were excluded from statistical analysis if the data from one or more samples were labeled as not detectable. When analyzing the statistical relationship between cell proportions identified using single cell RNA sequencing, the non-parametric Kruskal-Wallis test was conducted, followed by Dunn's post-hoc analysis. The Bonferroni correction was used to account for multiple hypothesis testing when performing the post-hoc analysis. When comparing the distribution of gene module scores across the three different culture conditions, effect size was used to compare large changes and was calculated as Cohen's d. Statistical details for each experiment can be found within each figure legend.

### Example 24: Reagents

A list of all reagents and resources used as described here, including source and catalog number, can be found in TABLE 4.

TABLE 4
Reagents and Resources

| | | |
|---|---|---|
| REAGENT or RESOURCE | SOURCE | CATALOG NUMBER |

Primary Antibodies (Dilution used)

| | | |
|---|---|---|
| Alexa Fluor 647-Conjugated Anti-Chromogranin A Antibody | Novus Biologicals | NBP2-47850AF647 |
| (1:100) | | |
| Alexa Fluor 647-Conjugated Mouse IgG2b kappa Isotype | Biolegend | 400330 |
| Control Antibody (1:100) | | |
| Anti-Chromogranin A Antibody (1:100) | Agilent/Dako | M086901-2 |
| Anti-Chromogranin A Antibody (1:100) | Millipore Sigma | HPA017369-100UL |
| Anti-Cholecystokinin Antibody (1:100) | Abcam | Ab27441 |
| Anti-GIP Antibody (1:100) | Invitrogen | PA5-76867 |
| Anti-GLP-1 Antibody (1:100) | Abcam | Ab23468 |
| Anti-PYY Antibody (1:50) | Mybiosource | MBS9208739 |
| Anti-Serotonin Antibody (1:100) | Abcam | ab66047 |
| Anti-Somatostatin Antibody (1:100) | R&D Systems | mab2358 |
| APC anti-human β2-microglobulin Antibody (1:25) | Biolegend | 316311 |
| APC anti-human CD298 Antibody (1:25) | Biolegend | 341706 |
| PE-conjugated Anti-Chromogranin antibody (1:100) | BD Biosciences | 564563 |
| PE-conjugated Mouse IgG1 kappa Isotype Control Antibody | BD Biosciences | 554680 |
| (1:200) | | |
| TotalSeq ™-B0251 anti-human Hashtag 1 Antibody (1:25) | Biolegend | 394631 |
| TotalSeq ™-B0252 anti-human Hashtag 2 Antibody (1:25) | Biolegend | 394633 |
| TotalSeq ™-B0253 anti-human Hashtag 3 Antibody (1:25) | Biolegend | 394635 |
| TotalSeq ™-B0254 anti-human Hashtag 4 Antibody (1:25) | Biolegend | 394637 |
| TotalSeq ™-B0255 anti-human Hashtag 5 Antibody (1:25) | Biolegend | 394639 |
| TotalSeq ™-B0256 anti-human Hashtag 6 Antibody (1:25) | Biolegend | 394641 |
| TotalSeq ™-B0257 anti-human Hashtag 7 Antibody (1:25) | Biolegend | 394643 |
| TotalSeq ™-B0258 anti-human Hashtag 8 Antibody (1:25) | Biolegend | 394645 |
| TotalSeq ™-B0259 anti-human Hashtag 9 Antibody (1:25) | Biolegend | 394647 |

Secondary Antibodies

| | | |
|---|---|---|
| Donkey anti-Goat IgG (H + L) Cross-Adsorbed Secondary | Invitrogen | A-11055 |
| Antibody, Alexa Fluor 488 (1:400) | | |
| Donkey anti-Mouse IgG (H + L) Cross-Adsorbed Secondary | Invitrogen | A-21202 |
| Antibody, Alexa Fluor 488 (1:400) | | |
| Donkey anti-Mouse IgG (H + L) Cross-Adsorbed Secondary | Invitrogen | A-31571 |
| Antibody, Alexa Fluor 647 (1:400) | | |
| Donkey anti-Rabbit IgG (H + L) Cross-Adsorbed Secondary | Invitrogen | A-31573 |
| Antibody, Alexa Fluor 647 (1:400) | | |
| Donkey anti-Rat IgG (H + L) Cross-Adsorbed Secondary | Invitrogen | A-21208 |
| Antibody, Alexa Fluor 488 (1:400) | | |

Chemicals and Enzymes

| | | |
|---|---|---|
| 4′,6-diamidino-2-phenylindole (DAPI) | Thermo Fisher Scientific | D1306 |
| A-8301 | Millipore Sigma | SML0788 |
| AS1842856 | Millipore Sigma | 344355 |
| B-27 Supplement | Thermo Fisher Scientific | 12587010 |
| Betacellulin | Peprotech | 100-50 |
| Bovine serum albumin | Millipore Sigma | 05470-1G |
| Cell Recovery Solution | Corning | 354253 |
| Cell Staining Buffer | Biolegend | 420201 |
| Collagenase Type I | Thermo Fisher Scientific | 17018029 |
| DAPT | Selleckchem | S2215 |
| Diprotin A | Tocris | 6019 |
| Advanced DMEM/F12 | Thermo Fisher Scientific | 12634028 |
| DMEM Ca2+ Free | Thermo Fisher Scientific | 21068-028 |
| RNAse Inhibitor | Thermo Fisher Scientific | N8080119 |
| EGF, Recombinant Murine | Peprotech | 315-09 |
| Fetal Bovine Serum Certified USA Origin | Thermo Fisher Scientific | 16000044 |
| Forskolin | Tocris | 1099 |
| Gastrin I [Leu15], Human | Millipore Sigma | G9145 |
| GlutaMAX | Thermo Fisher Scientific | 35050061 |
| HEPES | Thermo Fisher Scientific | 15630080 |
| Matrigel ®, growth factor reduced, phenol red-free | Corning | 356231 |
| N-2 Supplement | Thermo Fisher Scientific | 17502001 |
| N-Acetyl-cysteine | Millipore Sigma | A7250 |
| Nicotinamide | Millipore Sigma | N0636 |
| Noggin, Recombinant Murine | Peprotech | 250-38 |
| Normocin | Invivogen | ant-nr-2 |
| Paraformaldehyde, 32% | Electron Microscopy | 15714-S |
| | Sciences (VWR) | |
| PF-06260933 | Millipore Sigma | PZ0272 |
| Primocin | Invivogen | ant-pm-2 |
| Prolong Gold Antifade Mountant | Thermo Fisher Scientific | P36930 |
| Prostaglandin-E2 | Millipore Sigma | P0409 |
| Rimonabant | Millipore Sigma | SML0800 |
| SB202190 | Millipore Sigma | S7067 |
| SP600125 | Millipore Sigma | S5567 |
| Tranylcypromine | Tocris | 3852 |
| TRI Reagent ® | Millipore Sigma | T9424-200ML |
| Triton X-100 | Millipore Sigma | T8787-50ML |
| TruStain FcX ™ (Fc Receptor Blocking Solution), Human | Biolegend | 422301 |
| TrypLE ™ Express | Thermo Fisher Scientific | 12605-010 |
| Tubastatin-A | Selleckchem | S8049 |
| Y-27632 dihydrochloride | Tocris | 1254 |

Commercial Assays

| | | |
|---|---|---|
| 3′ Feature Barcode Kit, 16rxn Dual Indexing Compatible | 10X Genomics | 1000269 |
| Click-IT EdU Alexa Fluor 488 Flow Cytometry Assay Kit | Thermo Fisher Scientific | C10420 |
| Chromium NextGem Chip G Single Cell Kit | 10X Genomics | 1000127 |
| Chromium Next Gem Single Cell 3′ GEM, Library and Gel | 10X Genomics | 1000269 |
| Bead Kit v3.1, 4rxn Dual Index Compatible | | |
| Dead Cell Apoptosis Kit with Annexin V Alexa Fluor 488 & | Thermo Fisher Scientific | V13241 |
| Propidium Iodide | | |
| Dead Cell Removal Kit | Miltenyi Biotec | 130-090-101 |
| Direct-zol RNA Microprep Kit | Zymo Research | R2061 |
| High-Capacity cDNA Reverse Transcription Kit | Thermo Fisher Scientific | 4368813 |
| Human GIP (total) ELISA Kit | Millipore Sigma | EZHGIP-54K |
| Human GLP-1 (7-36) ELISA Kit | Abcam | Ab184857 |
| Human PYY ELISA Kit | Abcam | Ab255727 |
| Human Serotonin ELISA Assay Kit | Eagle Biosciences/DLD | SER39-K01 |
| | Diagnostika GmbH | |
| TaqMan ™ Universal PCR Master Mix | Thermo Fisher Scientific | 4304437 |

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. An in vitro generated three-dimensional gastrointestinal organoid derived from an induced human gastrointestinal stem cell (GISC), the organoid comprising functional human enteroendocrine cells.

2. The organoid of claim 1, wherein the enteroendocrine cells secrete hormones.

3. The organoid of claim 2, wherein the hormones are selected from the group consisting of: cholecystokinin (CCK), gastrin, ghrelin, glucagon-like peptide-1 (GLP-1), GLP-2, glucose-dependent insulinotropic polypeptide (GIP), histamine, leptin, motilin, neurotensin, oxyntomodulin, peptide YY (PYY), secretin, serotonin (5HT), and somatostatin (SST).

4. The organoid of claim 1, wherein the organoid comprises at least one of: enteroendocrine lineage markers, or any combinations thereof.

5. A method of enteroendocrine cell differentiation, comprising culturing gastrointestinal stem cells in a media and adding at least one modulating agent, wherein said modulating agent may be selected from a GATA4 activator, a cannabinoid type 1 receptor (CB1) inverse agonist, a direct or indirect PDX1 activator, a JNK inhibitor, a FOXO1 inhibitor, or combinations thereof, said modulating agent is in an amount effective to induce differentiation of the gastrointestinal stem cells, thereby resulting in enteroendocrine cell differentiation.

6. The method of claim 5, wherein the at least one modulating agent comprises a GATA4 activator and either a PDX1 activator or a JNK inhibitor.

7. The method of claim 5, wherein the at least one modulating agent comprises a FOXO1 inhibitor.

8. The method of claim 7, wherein the FOXO1 inhibitor is administered alone for a predetermined number days ranging 1-21 days.

9. The method of claim 8, further comprising administering for any number of days ranging 1-21 days a GATA4 activator and either a PDX1 activator or a JNK inhibitor, after FOXO1 is administered for the predetermined number of days.

10. The method of claim 8, further comprising administering for any number of days ranging 1-21 days a combination of a FOXO1 inhibitor, a GATA4 activator, and either a PDX1 activator or a JNK inhibitor, after FOXO1 is administered alone for the predetermined number of days.

11. The method of claim 5, wherein the media is a growth media comprising conditioned media containing Wnt3a, noggin, and R-spondin 3; mammalian cell culture media with high concentrations of glucose, amino acids, and vitamins with additional nutrients; supplements; buffering agent; antibiotic/antimicrobial; small molecule modulating agent; antioxidant; proliferating and/or differentiating agent; and hormone.

12. The method of claim 11, wherein said culturing occurs for greater than or equal to 1 day.

13. The method of claim 5, wherein the media is a differentiation media comprising conditioned media containing Wnt3a, noggin, and R-spondin 3; mammalian cell culture media with high concentrations of glucose, amino acids, and vitamins with additional nutrients; supplements; buffering agent; antibiotic/antimicrobial; small molecule modulating agent; antioxidant; proliferating and/or differentiating agent; hormone; growth factor.

14. The method of claim 13, wherein said culturing occurs for greater than or equal to 1 day.

15. The method of claim 5, wherein said culturing comprises: a first culturing step in a growth media for greater than or equal to 1 day, replacing the growth media with a differentiation media, and a second culturing step in a differentiation media for greater than or equal to 1 day.

16. The method of claim 5, wherein the at least one modulating agent is in a pharmaceutical composition, and at least one pharmaceutically acceptable carrier.

17. A method of treating a subject suffering from a disease having enteroendocrine cell dysfunction, comprising: administering to the subject in need thereof, a pharmaceutical composition comprising functional enteroendocrine cells in an effective amount to treat the subject and at least one pharmaceutically acceptable carrier.

18. The method of claim 17, wherein the functional enteroendocrine cells are derived from a gastrointestinal stem cell.

19. The method of claim 17, wherein the functional enteroendocrine cells are differentiated by the method of claim 5.

20. The method of claim 17, wherein the disease is a metabolic and/or gastrointestinal disease.

21. The method of claim 20, wherein the disease is selected from the group consisting of: obesity, type II diabetes mellitus, irritable bowel syndrome (IBS), inflammatory bowel disease (IBD), Crohn's disease, and ulcerative colitis.
